(19) United States
(12) Patent Application Publication
(10) Pub. No.: US 2012/0221075 A1
(43) Pub. Date: Aug. 30, 2012

(54) COMPUTERIZED SYSTEM OR DEVICE AND METHOD FOR DIAGNOSIS AND TREATMENT OF HUMAN, PHYSICAL AND PLANETARY CONDITIONS

(75) Inventor: Jonathan Bentwich, Zipori (IL)

(73) Assignee: Y.K.L. Brain Tech Ltd., RaAnana (IL)

(21) Appl. No.: 13/505,511

(22) PCT Filed: Nov. 2, 2010
(86) PCT No.: PCT/IL10/00912

§ 371 Date: May 2, 2012

Related U.S. Application Data
(60) Provisional application No. 61/272,779, filed on Nov. 2, 2009.
(60) Provisional application No. 61/282,635, filed on Mar. 10, 2010.
(60) Provisional application No. 61/344,024, filed on May 10, 2010.

(51) Int. Cl. A61N 1/36

(52) U.S. Cl. 607/45

(57) ABSTRACT

The invention outlines a system, method or device capable of modulating the planetary, physical, and brain related aspects of human existence through the combination of diagnostic and corrective or therapeutic or stimulation modalities that are capable of identifying and evaluating any imbalances or pathological conditions or abnormal conditions pertaining to the environment, brain condition, or physical conditions of human beings and applying corrective or stimulation modalities or influences that are capable of improving or optimizing or healing or otherwise balancing these planetary, physical, human, brain related or bodily aspects or measures or conditions.

An implantable (and/or non implantable) ‘Brain Optimizer’ neuronal stimulator system aimed at optimizing neurophysiological brain functions associated with different brain diseases (or enhancing normal brain functioning or traits) such as: Parkinson's, Depression, Stroke etc. is described which is capable of adjusting and correcting abnormal neurophysiological brain function associated with any brain related disease. The Brain Optimizer achieves this goal of optimizing brain activity by comparing the neurophysiological brain functions (or brain activation, structure of function etc.) associated with a particular disease and/or with a particular and/or individual with the corresponding norm for neurophysiological activation pattern (structural and/or functional etc. other brain properties—associated with any particular region/s and/or disease and/or individual), which is then utilized for purposes of adjustment of neurophysiological, electromagnetic or electrical etc. stimulation parameters of the implantable (or non-implantable) stimulating components that stimulate single or multiple brain loci associated with the disease and/or individual (e.g., also for purposes of enhancement of brain functioning in normal individuals). The methodology of implantation of the Brain Optimizer (e.g., single or multiple electrodes, coils, chips or any other invasive or non-invasive stimulating component/s) is also claimed which is based on a functional analysis (‘real-time’ or ‘off-line’) of the neuronal response of a single or multiple brain loci, neuron/s etc. to particular cognitive or electrical/electromagnetic stimulation—that is calibrated and/or fine-tuned against the targeted brain function, neurophysiology or functional (or structural) properties of a norm based criteria (e.g., of normal brain functioning and/or disease-specific brain loci/locus or other norms and/or of enhanced brain functioning). Such implantation (or non-invasive) brain stimulation methodology and/or device allows for the identification and placement of the Brain Optimizer stimulating components precisely at the right localization needed to effectively stimulate the aberrant or abnormal or dysfunctional or impaired brain function (which can be disease-specific and/or individual-based) or to enhance brain functioning in normals due to the fact that it interactively or iteratively measures an adjusts the localization and/or loci/locus of brain stimulation (at single or multiple brain localizations) and/or the stimulation parameters—e.g., in terms of electrical/electromagnetic brain stimulation (intensity, duration, timing, synchronization, even-related and/or function-specific and/or criteria-based), and/or cognitive or behavioral or neurophysiological stimulation and/or neurophysiological or brain activation response to such electrical/electromagnetic and/or cognitive or behavioral or any other brain function stimulation. Such stimulus-response adjustment and/or fine-tuning of the localization, dynamics, stimulation (electrical/electromagnetic and/or cognitive/behavioral/neurophysiological) of brain loci/locus based on neuronal response and/or norm-based criteria (e.g., for normal healthy brain functioning and/or for diseased brain functioning associated with a particular disease and/or individual and/or enhanced or enhancement of any brain function, intelligence or functioning or any other desired or criteria-based optimal and excelled brain functioning) allows the Brain Optimizer to modulate, regenerate, enhance or correct or optimize the specific functioning, structure or activation of any impaired brain region/s and/or disease and/or individual (in real-time or off-line) until it reached a given criteria (health-based, functioning-based, structure, function, neurophysiological based etc.) which can therefore effectively treat, regenerate, heal, enhance any brain related function and/or disease and or in a given individual. The structure and/or operation of the Brain Optimizer is claimed to be such that it can consist of implantable single or multiple and/or cortical and/or sub-cortical and/or electrodes and/or stimulators and/or electrical/electromagnetic and/or coils which can operate with or without concurrent and/or convergent and/or synchronized and/or cognitive and/or behavioral and/or any other brain or behavior stimulation and/or manipulation and/or until a particular brain and/or behavior and/or disease modification and/or enhanced and/or improved brain functioning etc. and/or on an on-going electrical/electromagnetic and/or cognitive/behavioral/neurophysiological stimulation. Finally, the construction and/or design of the Brain Optimizer is such that it can consist of single or multiple sensors or measurement/s of single or multiple brain region/s which can be synchronized and/or convergent and/or independent of the electromagnetic and/or electrical and/or cognitive and/or behavioral stimulation protocol—which can allow for real-time or off-line optimization and/or enhancement and/or regeneration and/or effective treatment of any brain related disease or condition and/or in an individual etc.

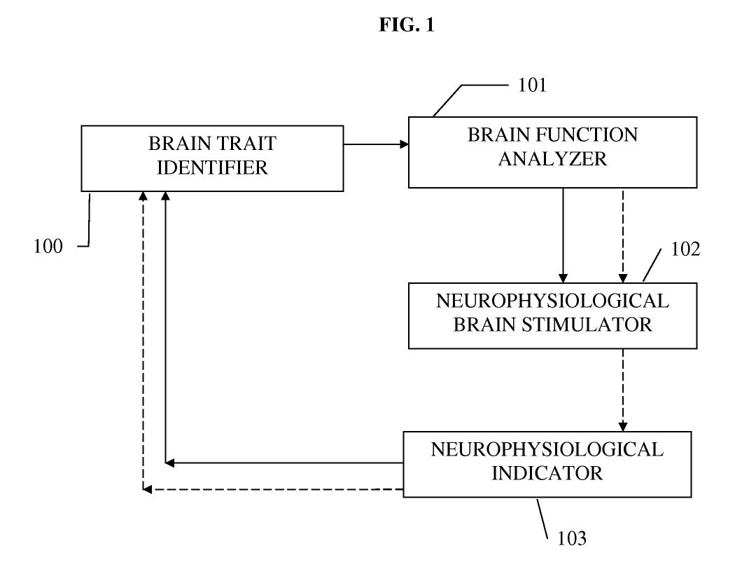

Reference is now made to FIG. 1 which describes a NEUROPHYSIOLOGICAL INDICATOR 103 which is capable of measuring and/or assessing and/or indicating and/or at single or multiple time-points and/or for single and/or multiple brain region/s loci/locus various and/or all brain function/s structural and/or cognitive value/s and/or neurophysiological values and/or characterizations. The NEUROPHYSIOLOGICAL INDICATOR 103 informs and/or outputs information or measurement/s (relating to all brain measurements and/or described above) to the BRAIN TRAIT IDENTIFIER 100 which is a computational and/or decision-making and/or algorithm-based and/or automatic and/or non-automatic and/or physician-aided module and/or element and/or processing stage etc. capable of determining which brain region/s and/or cognitive and/or behavioral and/or neurophysiological traits and/or characteristics and/or values, functions, patterns and/or function and/or structure and/or neurophysiological trait/s etc. are in need of change and/or treatment and/or regeneration and/or enhancement and/or in an individual and/or in a particular disease and/or related to a single and/or multiple brain region/s or loci/locus and/or are impaired and/or diseased and/or require enhancement—e.g., whether related to a particular disease and/or brain condition and/or enhancement of brain function/s and/or intelligence and/or individually-based and/or adjusted according to a particular health-norm and/or brain functioning criteria etc. The BRAIN TRAIT IDENTIFIER 100 determines the targeted brain functions and/or cognitive and/or behavioral and/or neurophysiological traits and/or values and/or criteria and/or criterion that need to be altered and/or enhanced and/or normalized and/or treated and/or improved and/or enhanced etc. and/or associated with a particular brain region and/or regions and/or functional and/or structural and/or neurophysiological patterns and/or values and/or targets and/or criteria/criterion. BRAIN TRAIT IDENTIFIER 100 then transmits and/or outputs and/or informs the BRAIN FUNCTION ANALYZER 101 which of the single and/or multiple and/or brain loci, locus or region/s or neuron/s and/or which cognitive and/or behavioral and/or neurophysiological stimulation/s and/or manipulation and/or neuronal modulation and/or stimulation and/or enhancement and/or treatment should be applied in order to achieve the criteria established and/or determined by the BRAIN TRAIT IDENTIFIER 100; Hence, the BRAIN FUNCTION ANALYZER 101 precisely determines any electromagnetic and/or electrical and/or cognitive and/or behavioral and/or any other neurophysiological and/or neuronal stimulation and/or stimulus and/or stimuli and/or manipulation and/or intervention and/or treatment should be employed and/or carried out and/or for which brain region/s and/or single and/or multiple locus or loci and/or what stimulation (electromagnetic/electrical and/or cognitive and/or behavioral) and/or stimulation parameters and/or duration and/or intensity and/or synchronized and/or non-synchronized stimulation format should be utilized and/or in order to achieve the criteria and/or treatment and/or clinical and/or brain enhancement function/s and/or single or multiple brain region functioning and/or values and/or performance and/or structure and/or function and/or cognitive goal and/or criteria/criterion (as determined by the BRAIN TRAIT IDENTIFIER 100). These outputs of the BRAIN FUNCTION ANALYZER 101 are then transmitted and/or outputted to the NEUROPHYSIOLOGICAL BRAIN STIMULATOR 102 which is responsible for executing the appropriate (determined) electrical and/or electromagnetic and/or neurophysiological and/or cognitive and/or behavioral stimulation parameters and/or duration and/or intensity and/or single and/or multiple and/or brain region/s and/or loci/locus etc. that are necessary in order to attain the criteria etc. set by the BRAIN TRAIT IDENTIFIER 100; Hence, the NEUROPHYSIOLOGICAL BRAIN STIMULATOR 102 administers electrical and/or electromagnetic and/or neurophysiological and/or cognitive and/or behavioral stimulation parameters and/or duration and/or intensity and/or single and/or multiple and/or brain region/s and/or loci/locus etc. stimulation to the brain region/s and/or brain function/s and/or cognitive and/or structural and/or function/s of the brain.

Following the NEUROPHYSIOLOGICAL BRAIN STIMULATOR 102 delivery of single and/or multiple brain stimulation (e.g., as described above electromagnetically and/or cognitively and/or neurophysiologically etc.) it outputs or informs or otherwise interacts with the NEUROPHYSIOLOGICAL INDICATOR 103 which measures post-stimulation and/or intermittent and/or at various time- and/or spatial brain points) measurements as described above of all brain functions, structure, cognition, neurophysiological etc. measurements and/or in ‘real-time’ and/or ‘off-line’, which are then fed back to the BRAIN TRAIT IDENTIFIER 100 to determine whether there has been any change in brain function and/or cognition and/or behavioral and/or neurophysiological values and/or criteria/criterion and/or target values etc. Based on this secondary analysis of the BRAIN TRAIT IDENTIFIER 100 which compares between the post-stimulation (or intermittent-stimulation or any post-first stimulation time- and/or spatial brain point/s measurement) brain measurements (as described above including structural and/or, functional and/or cognitive and/or neurophysiological and/or other measurements) and the initial BRAIN TRAIT IDENTIFIER 100 determination of the target and/or criteria-based and/or NEUROPHYSIOLOGICAL INDICATOR 103 initial measurement/s—which can all be stored within the BRAIN-TRAIT IDENTIFIER 100 the BRAIN TRAIT IDENTIFIER 100 computes and outputs to the BRAIN FUNCTION ANALYZER 101 the adjusted and/or modified criteria or values of stimulation (e.g., for the single or multiple brain region/s and/or cognitive and/or behavioral and/or neurophysiological stimulation parameter/s) which then serve as output for the execution of a new and/or adjusted and/or single or multiple brain region/s and/or cognitive and/or behavioral and/or neurophysiological stimulation parameter/s; This sequence is continued until the criteria or criterion or targeted brain value/s, structure, function or cognition or any other trait determined (initially) by the BRAIN TRAIT IDENTIFIER 100 is attained.

Thus, for instance, the system and/or medical device and/or methodology and/or method (described above) can include a measurement by the BRAIN TRAIT IDENTIFIER 100 of deficient and/or abnormal electrophysiological and/or EEG and/or ERP and/or functional brain activation (such as for instance measured in fMRI, PET, SPECT etc.) structural abnormality/ies that may be characteristic of a particular brain disease such as Parkinson's or Depression or Stroke (at single or multiple brain region/s loci/locus functions etc.), Autism, Aphasia, Traumatic Brain Injury, PTSD etc. (excluding AD) and based on BRAIN FUNCTION ANALYZER 101 determination of the appropriate stimulation parameter/s as described above in order to reach a particular value, criteria, health-related norm, functional activation standard (per age, disease, regeneration of lost function/s etc.) which then instructs the NEUROPHYSIOLOGICAL BRAIN STIMULATOR 102 as to how to stimulate (as described above—electromagnetically/electrically and/or cognitively and/or neurophysiologically or neuronally) the relevant and/or appropriate brain region/s and/or structure/s and/or function/s etc. Following such integrative and/or dissociative electrical/electromagnetic and/or cognitive and/or behavioral stimulation of the appropriate brain region/s and/or loci/locus and/or diseased and/or impaired and/or region/s and/or loci/locus and/or intermittently and/or synchronized with such single or multiple spatial/temporal brain stimulation of the diseased region/s that are specific for a particular brain disease/s and/or a particular individual and/or clinical population/s—there can be a measurement by the NEUROPHYSIOLOGICAL INDICATOR 103 of different electrophysiological and/or, functional, and/or cognitive values and/or at different time and/or spatial (brain) points and/or regions etc. which is then transferred to the BRAIN TRAIT IDENTIFIER 100 and to the BRAIN FUNCTION ANALYZER 101 in order to compare the measured electrophysiological, cognitive, structural or behavioral values or change relative to previous measurements or relative to the health and/or clinical and/or diseases and/or criteria norms. Such comparison to the target electrophysiological and/or cognitive etc. norms or criteria etc. can then be utilized in order to determine and/or compute the adjustment in electromagnetic and/or electrical and/or cognitive and/or behavioral stimulation parameters necessary to achieve and/or attain the desired goal or criteria for improved and/or enhanced or therapeutic treatment of any particular brain disease/s and/or condition etc. This feedback loop can be employed multiple times until the desired criteria has been achieved.

Implantation of Electrodes at Precise Functional Locus/i, Brain Optimizer possible embodiment and/or principles of operation: One possible embodiment (and claim) of this invention is a BRAIN STIMULATOR for instance for treating Parkinson's disease which consists of single and/or multiple electrodes and/or coils and/or implantable or non-invasive electrical or electromagnetic stimulating elements (including but not limited to chips, electrodes, electromagnetic coils, electrical inducing wires etc.) which can be implanted at the precise brain diseased region/s that are particular to Parkinson's disease and/or for a particular patient with Parkinson's. These disease- and/or individual and/or stage-specific impaired (or for enhancement of intelligence those brain region/s that wish to be enhanced corresponding to particular cognitive or behavioral etc. functions desired to be enhanced or optimized etc.) can be determined either based on a comparison of the functional and/or cognitive and/or structural and/or neurophysiological values and/or patterns and/or characterization/s of the individual- and/or patient/s in a particular disease such as PD with health norms and/or disease norms and/or given criteria etc. and/or or can be gathered from known scientific knowledge and/or new experiments conducted to determine pre-clinical and/or symptomatic and/or clinical characterization of the disease.

According to this embodiment, the electrodes or stimulating elements (invasive or non-invasive) can be implanted (or placed) at the appropriate (precise) brain loci/locus which can be determined anatomically and/or structurally and/or cognitively and/or electro physiologically—such as for instance through a (partial and/or full) anesthetized procedure which is capable of measuring the specific brain activation and/or specific loci/locus response to relevant particular stimulus/i (e.g., electromagnetic/electrical and/or cognitive and/or behavioral and/or electrophysiological and/or neurophysiological etc.); Thus for instance there exists one such known surgical procedure with partial anesthesia for verbal functioning whereby the surgeon implants or places the electrode/s on Broac's region and stimulates this region (in particular manner/s) while the patient is attempting to vocalize or verbalize certain utterances or verbal tasks—until the patient's capacity to speak is impaired or halted- which implies that the precise regions/s for that particular verbal function has been stimulated (and localized). This known localization methodology can be alternated with the suggested comparison and identification of the precise functional, structural, cognitive and/or electrophysiological abnormal and/or impaired brain region/s loci/locus etc.—in any format that allows to precisely identify the diseased and/or impaired brain region/s loci and/or locus etc. that needs to be stimulated.

Combined Electrophysiological & Electromagnetic/Electrical & Cognitive Stimulation Configuration: One possible configuration (and claim) of the suggested embodiment and/or invention is the integrative placement of electromagnetic and/or electrical stimulating elements and/or component/s along with Neurophysiological Indicator sensing elements, such as electrodes measuring EEG, ERP and/or any other form of measurement of electrical and/or electromagnetic activity and/or values and/or pattern/s etc.—such that it allows for ‘real-time’ or off-line synchronized and/or independent and/or serial and/or parallel and/or single and/or multiple spatial-temporal electrophysiological measurements of the same and/or adjacent and/or any other spatial-temporal brain loci/locus and/or single and/or multiple brain region/s point/s measurement/s.

A Brain-Medic Integrative Diagnostic and therapeutic system for personalized diagnosis and treatment or enhancement of any brain related disease or condition or enhancement is an integrative diagnostic and therapeutic personalized system capable of early screening, diagnosis and treatment of any brain-related disease through the integration of off-line and real-time individual-based diagnosis of disease-specific brain regions, structure, function, cognition behavior or neurophysiology together with a convergent stimulation of non-invasive (or invasive) electromagnetic or electrical stimulation together with personalized and interactive (learning system) cognitive or behavioral stimulation of the identified disease-specific (personalized) regions synchronized and adapted personally to optimize neurophysiological regenerative neuroplasticity effects, behavioral or cognitive learning, encoding or regeneration of deficient, lost, or diseased brain functions. The integrative BRAIN-MEDIC medical device/s system can consist of an (early) diagnosis system which can consist of a Personalized Neuroimaging Device/s (method or system which may be called: ‘Hope Detector’) capable of deriving an individual's specific diseased, deficient or abnormal (structure, function, cognition/behavioral or neurophysiological) brain regions and corresponding brain functions or cognitive/behavioral measures and/or a cognitive/behavioral Brain/Disease Predictive Analyzer capable of predicting or providing a reliable measure of the detection of specific brain diseases or abnormalities in an individual by the Personalized Neuroimaging Device; The BRAIN-MEDIC system also consists of a therapeutic personalized Brain-Stimulation device which can consist of an integrative (off-line) input from the Hope Detector identifying the specific diseased brain regions (in an individual) that are then inputted into a non-invasive (or invasive) brain stimulation system that is co-registered precisely to stimulate the identified brain diseased regions (identified by the Hope Detector) and which can measure (in ‘real-time’) neurophysiological responses to focal single/multiple brain loci stimulation, cognitive or behavioral responses and learning—which can then also inform the third component of the BRAIN-MEDIC system which is the Computerized Personalized Cognitive Stimulator that delivers individual-based disease specific cognitive or behavioral stimulation synchronized with the focal electromagnetic stimulation of particular single or multiple (sequenced, synchronized or unrelated) brain loci or regions. The BRAIN-MEDIC System is capable of screening, detecting, or otherwise identifying any brain related disease prior to the emergence of disease-specific symptom or identifying the diseased brain regions in an already diseased patient (in a particular disease/s) and of stimulating the identified individual-based diseased brain regions both electromagnetically/electrically and cognitively in order to produce regenerative therapeutic neuroplasticity effects, through the combination of off-line screening and identification of the diseased brain regions by the Hope Detector and a real-time convergent synchronized electromagnetic/electrical and cognitive or behavioral stimulation of specifically diseased or impaired brain or cognitive functions.

The Hope Detector component of the BRAIN-MEDIC System (which can function as a ‘stand-alone’ or operate as part of the entire BRAIN-MEDIC integrative personalized diagnostic and therapeutic system) operates through a complex set of neuroimaging, cognitive, behavioral, neurophysiological or any other brain related measure and powerful computational, analytical, algorithmic etc. means for determining disease-specific brain, cognitive, neurophysiological, functional etc. abnormalities indices or for predicting the development or presence of a specific brain abnormality, function/s or disease/s; As stated above the Hope detector can possibly have two or more components which can include a Personalized Neuroimaging Device capable of identifying, screening or detecting diseased or impaired or abnormal brain regions or functions, structure, cognition, behavior etc. (and providing continuous feedback measurement and adjustment computation following any regimen of disease-specific and individual-specific brain stimulation protocol, regimen, interval etc.), and a cognitive or behavioral or analytical/computational component (termed: Cognitive Predictor) which possesses the capacity to predict or determine or associate reliably the presence of any of these abnormal brain disease/s in an individual through a ‘simpler’, ‘quicker’ or less complex procedure/s of administering cognitive, computerized or behavioral or neurophysiological or other ‘simpler/less complex’ operations than the full Personalized Neuroimaging Device operation/s (described above). One good example for the operation or function of the Cognitive Predictor is the potential capacity of a computerized cognitive testing or tool which measures specific cognitive, behavioral or neurophysiological etc. measures to predict or detect or identify the presence of Parkinson's disease through for instance a set of motor, cognitive, affective, behavioral or neurophysiological measures—thereby predicting with a certain probability or reliability the fuller (and potentially more accurate or reliable) determination of the presence, development or indication of Parkinson's disease in a given individual; Thus, the Cognitive Predictor may be capable of detecting the presence or development of a specific disease/s in an individual (such as for instance the case of Parkinson's, Depression, Stroke, Autism, ADHD, Traumatic Brain Injury, PTSD, Schizophrenia, Multiple Sclerosis, Aphasia, or by extension any other Bodily Disease which may be correlated or associated with specific Brain dysfunction/s, cognitive or behavioral correlates or measures that may be correlated, associated or otherwise indicative of such bodily disease or dysfunction etc., or a certain brain/cognition/behavioral/neurophysiological etc.) through the administering of a rather ‘simple’, time-effective or less complex procedure such as for instance a computerized test or automated analysis of certain neurophysiological measures (such as EEG, ERP etc.) with/without other computerized cognitive tests or measures then the fuller Personalized Neuroimaging Device—and may actually predict with relatively high probability or reliability the existence or development of the disease identified clinically or through the Personalized Neuroimaging Device measures or determination. Thus, it may be possible for an individual to undergo a ‘quick’ or (relatively) ‘simple’ Cognitive Predictor test/s which would screen the general population or the relevant at-risk or disease-specific population for the presence or development of a particular disease/s in an individual such that would be perhaps less reliable than the Personalized Neuroimaging Device but which still provide an initial robust or reliable means for determining the presence or development of a particular disease/s or dysfunction in the brain or Body, or alternatively can allow for the detection of a particular brain dysfunction, locus or loci, particular cognitive or behavioral disease, impairment or dysfunction etc.—that can later on be screened or identified or detected through such means as the Personalized Neuroimaging Device.

The operation or function or principles underlying the Cognitive Predictor can consist of the identification of specific cognitive or behavioral or neurophysiological measures that correlate or can be associated or reliably predict specific CSF, neurophysiological, or Personalized Neuroimaging Device or any other structural, functional or cognitive/behavioral measure of brain dysfunction or specific disease/s or specific brain abnormality (localized at single or multiple brain regions or spread systemically or at specific brain systems or circuitry etc.)—indices of a particular disease/s, dysfunction brain abnormality/ies. Thus, for instance, to the extent that a particular cognitive measure or test or behavioral. Motor, neurophysiological or other single or multiple measures can predict reliably or with relatively high accuracy or association etc. a specific biomarker for a specific disease, brain dysfunction, brain or cognitive or behavioral or neurophysiological loci/locus of injury or impairment, then this single or multiple Cognitive Predictor index/indices may become potent predictors of the existence or development of the disease;

Therefore the usage or method or system or operation of the Cognitive Predictor (as a stand-alone or as part of the BRAIN-MEDIC system) involves a certain cognitive, behavioral, neurophysiological or other brain or cognitive or behavioral etc. measure or index—whether developed proprietarily or selected and analyzed/computed as a predictor or indicator of a particular brain disease/s, condition, brain locus/loci impairment—if shown to reliably predict or be associated etc. with that particular disease, brain function, cognitive or behavioral or neurophysiological impairment then this indicator, measure, index is claimed as an effective Cognitive Predictor for that disease or condition, and may be utilized as a proprietary screening or general population or at risk or patient population initial test or identification of that particular disease.

Likewise the particular analytical or computational method, procedure or device is described whereby particular brain disease, dysfunction, brain loci/locus abnormality or illness (or brain-related dysfunction indices of bodily diseases as explained above) is predicted, identified or otherwise detected through the identification of a specific set of single/multiple brain (loci/locus, function, structure, cognitive or behavioral etc.) measures; This analytical/computational methodology can rely on the identification or analysis or derivation of certain values, abnormalities, thresholds, components, scores or specific patterns of the abovementioned Cognitive Predictor assessed cognitive, behavioral, neurophysiological measure/s that indicate a high association, correlation or predictive value for the acceptable clinical diagnostic, brain (CSF etc.) biomarkers or the Personalized Neuroimaging Device identification or assessment of a particular disease, or specific brain dysfunction, loci. Locus etc.; Thus, for instance, a claim can be made for a methodology or device etc. in which a measure of a particular cognitive or behavioral measure is found or computed or assessed to be associated or reliably predictive of the presence or development of a particular disease (such as Parkinson's, Depression etc.)—through for example a determination of a particular threshold, value or pattern of this single or multiple test/s or single or multiple items or components of pattern/s within a given test/s, index/indices or series of cognitive, behavioral or neurophysiological measures (values, thresholds, patterns etc.); Such methodology can also be claimed to predict or be utilized as a predictive or screening methodology for convergent or later usage of other more reliable disease-specific identifiers such as the Personalized Neuroimaging Device etc.

Likewise the Cognitive Predictor can be utilized in order to ‘hone’ in or to selectively identify particular brain-diseased region/s or cognitive or behavioral abnormality/ies or dysfunction/s etc.—also as an initial screening means or early detection device that can be used as a ‘stand-alone’ or as a precursor or as a (preliminary) aid to the fuller or more definitive clinical or Personalized Neuroimaging Device or other neurophysiological, pathological or clinical or other more precise measures of specific brain loci/locus, function, cognitive or behavioral dysfunction etc.

The Personalized Neuroimaging Device can consist of an (‘off-line’ or ‘real-time’) integration of a series of brain, neuroimaging, structural, functional, cognitive or behavioral or neurophysiological etc. measures—which are contrasted against and compared, analyzed relative to normative clinical data of disease or healthy norms to determine disease-specific threshold/s or critical or indicative value/s such that a particular set of indicators, values or measures related to single or multiple brain region/s and/or cognitive, behavioral, neurophysiological patterns is capable of predicting or assessing the existence of a particular brain disease/s, condition, abnormality etc.

Hence, the Personalized Neuroimaging Device can potentially serve to screen and detect in the general population or specific, at risk, or specific clinical population/s the occurrence, presence or development of specific brain illness/es—with particular brain region/s loci/locus abnormalities or cognitive or behavioral or neurophysiological abnormalities, values, or deficiencies or impairments (relative to clinical or other normative indices) indicative or predictive of particular brain disease/s; Thus, the Personalized Neuroimaging Device may be capable of screening an individual or a given population (e.g., specific clinical population/s or the general population/s etc.) for a particular disease/s or single or multiple diseases or impairments or specific brain loci/locus abnormalities or dysfunction/s etc.; Different scenarios, usages or clinical or therapeutic strategies can be envisioned and operated therefore with the Personalized Neuroimaging Device described wherein an individual/s (or a population/s) are either selectively screened (or targeted) for the detection of a particular brain disease/s and/or specific brain loci or locus impairment or are screened or diagnosed for a variety, range or brain related diseases (or bodily diseases as described above). According to such possible scenario/usages an individual/s or a population may be screened or diagnosed for a variety of brain-related disease/s or can be specifically screened or tested for the existence or development of a particular brain region, loci, locus of impairment, illness or deficiency.

Therefore, the Personalized Neuroimaging Device possesses the capacity to determine specific brain loci, locus, single or multiple brain region/s impairment, illness, or deficiency and/or associated cognitive or behavioral or neurophysiological or any other brain related deficiency or impairment based on a comparison of that particular brain region/s cognitive or behavioral patterns, values or performance to established healthy or clinical patient population—which can determine the existence (or development) of a particular disease/s, illness/es, brain loci/locus impairment/s in an individual.

Such computational determination can be based on an integration of single or multiple brain, structural, functional, cognitive or neurophysiological or other brain related indices which are each compared to a norm of healthy or diseased individuals/population/s such that each of these indices (individually or collectively) indicates an above- or below-threshold/s or critical value/s that are associated with clinical diagnosis of a particular disease or impairment or alternatively are associated with functional, structural, cognitive impairment characteristic of a particular brain disease/s;

One specific configuration of the computational methodology underlying the Personalized Neuroimaging Device may consist of the determination of specific threshold value/s for each of the characteristic structural, functional, cognitive, behavioral, or neurophysiological, neuroplasticity, learning/encoding, cognition measures or specific vulnerable brain region/s associated with a particular disease/s or brain dysfunction/s—e.g., with each of these statistical/computational threshold/s being predictive or reliably associated with high probability or accuracy of the existence or prospective development of a particular disease/s or illness/es or brain dysfunction/s; One example for such computational methodology or principle/s underlying the Personalized Neuroimaging Device can be the statistical or computational identification of threshold or critical (reliable/predictive) threshold for the structural, functional, cognitive or neurophysiological value/s of a specific patient population/s (such as Parkinson's, Depression, Stroke, Autism, etc.) above- or below- which there exists a 95% (or any other determined reliable predictive value/s) for an individual possessing, exhibiting or developing a particular disease/s or brain loci/locus impairment/s.

Alternatively, the configuration of the Personalized Neuroimaging Device computational configuration may be based on a consortium, series or combination of the abovementioned structural, functional, cognitive or neurophysiological etc. factor/s, threshold value/s that can collectively predict or reliably detect with high probability the existence or prospective development of a particular disease/s illness, specific brain deficiency/, loci/locus, etc.; One example for such computational analysis utilizing a consortium, series of factors can be a formula that takes into consideration different predictive values, weights, or values or combination of the various factors as predicting with a certain probability or reliability the existence of a particular brain disease/s or impairment or brain region/s. Such computational series can also yield an algorithm of decision procedure or rule/s by which is it possible to determine whether or not an individual is diagnosed or possesses a certain disease/s or the likelihood of a person developing a particular brain disease, disorder, illness, deficiency or impairment/s (based on particular structural, functional, cognitive or neurophysiological value/s, thresholds, algorithm, decision rule/s).

The BRAIN-MEDIC therapeutic system consists of an (off-line) Hope Detector (which can consist of the Personalized Neuroimaging Device and/or the Cognitive Predictor) that yields a specific single or multiple brain region/s which are indicative or diagnostic or can screen for the existence or prospective development of a particular disease/s or specific brain, loci/locus dysfunction etc.—and which then can automatically instruct or determine or control the specific brain region/s to be stimulated electromagnetically or electrically and also cognitively; The operation of the BRAIN-MEDIC system is based on a co-registration of the Hope Detector's identification of the single or multiple diseased brain region/s associated with a particular disease/s and/or the associated cognitive or behavioral dysfunction/s which are identified in a particular individual—that is then co-registered to the individual's brain and also to the BRAIN-MEDIC stimulator's specific stimulation of the identified diseased or impaired brain region/s in an individual (and is also coordinated or automatically configured with computerized cognitive stimulation); Hence, the BRAIN-MEDIC system consists of an off-line screening, diagnosis and detection of the diseased brain region/s in an individual and/or that may be associated with a single or multiple brain related disease/s—which are then utilized (automatically or non-automatically) for the purpose of determining and controlling the invasive or non-invasive brain stimulation and convergent computerized cognitive stimulation of the identified single/multiple diseased brain region/s; These identified individual-based diseased brain region/s are then co-registered to the individual's BRAIN-MEDIC Stimulation Device (referred to earlier as the BRAIN-MEDIC therapeutic device) which then allows for a single or multiple sessions or treatment interval/s that are appropriate and specific to that individual and/or disease and/or at a particular point in time, and/or phase of the disease and/or specific brain loci/locus and/or specific cognitive or behavioral or structural or functional or neurophysiological; It should be noted that the Hope Detector (and/or any of its components described earlier) allows for pre- and post-treatment of the Brain Stimulation Device in any given individual/s patient population of disease type and can change across different brain related diseases and/or phases and/or individual/s such that the brain stimulation parameters (e.g., electromagnetic stimulation, cognitive stimulation, intensity, duration number of electrical/electromagnetic pulse/s and/or cognitive stimulus/stimuli, timing synchronization etc.) can be adjusted on an individual basis specifically for each session, series of sessions, phase of the disease and can even be adjusted interactively within a given session/s based on the individual's performance, neurophysiological measures, structural, functional or other measures. The BRAIN MEDIC stimulation device also allows for modulation of neuronal and/or cognitive and/or behavioral activity or performance etc. through its electrical or electromagnetic engineering, or design which essentially allows for real-time (synchronized or time-sensitive) localized and co-registered measurement of the relevant diseased or impaired brain region/s and/or associated cognitive or behavioral functions, performance etc.; Specifically, time-locked synchronized electromagnetic or electrical stimulation of diseased or impaired brain region/s, loci or locus that is synchronized with the presentation, training, stimulation and/or response that are associated or specific for a particular brain region/s allows for the restoration, alteration, regeneration or otherwise neuroplasticity or therapeutic effects that are specific to a particular disease/s and/or region/s and/or individual. Another aspect of the BRAIN MEDIC system (invention) is the capacity to adapt and adjust the brain stimulation (electromagnetic and/or cognitive) to the individual's level or learning, performance, impairment and learning or advancement curve through real-time interactive assessment and modulation of the level, intensity, duration, number of pulses and cognitive stimuli etc. that are administered to this individual in a particular session/s or within such session/s or throughout a course of treatment session/s etc. geared towards optimizing the stimulation parameters to produce optimal individual based and/or disease-specific (and/or phase, time, dynamics' specific) electro/magnetic and convergent cognitive or behavioral stimulation. Indeed, one possible configuration of the BRAIN-MEDIC system allows for the construction of the BRAIN-MEDIC (therapeutic) Stimulator in such a manner that combines between a (non-invasive or invasive) helmet configuration capable of stimulating single or multiple cortical or sub-cortical brain region/s that is closely coupled with real-time EEG, ERP or any other advanced methodology for measuring electrophysiological brain activity (with or without advanced structural of functional neuroimaging capabilities or device/s). Such configuration would allow for a close monitoring and potential modulation of the neuronal and/or cognitive or behavioral processing or activity that is associated with the diseased brain region/s or neurophysiology- through a measurement of the individual-based/and/or diseased-based specific diseased or impaired brain region/s, loci, locus etc. neuronal, electrophysiological or other structural or functional activity or values, characteristics etc.; Specifically, the combination of electrophysiological, ERP, EEG or functional (or structural) neuroimaging or other focal (real-time or off-line) measurements of the individual- and/or disease-specific brain diseased or impaired brain regions would allow for a few very valuable and potentially very significant applications and interventions including: the capacity to synchronize the electromagnetic or electrical activity with any cognitive stimulation of the same affected or impaired brain regions—such as for instance measurement of the timing of the electromagnetic/electrical pulse/s precisely before/after/simultaneously with the neuronal activity in the diseased or targeted brain region/s, or even very precise synchronization and/or stimulation of specific (affected or targeted) brain region/s at very particular time course points or aligned/synchronized (before/after or simultaneously with) specific electrophysiological or functional activation elements or components or characteristics which can also be associated with particular neuronal, cognitive or brain related activities, functions or events (such as for instance the capacity to stimulate at single or multiple loci/locus and single/multiple time points in each of these stimulated brain regions—associated with specific language, memory, perception or any other brain cognitive or behavioral function/s or time courses associated with specific impairments and/or brain loci; Or the application of such single/multiple brain- and time-point/s stimulation in relationship with the presentation of particular cognitive, behavioral, or electrophysiological stimulation synchronized in such a manner as to optimize neuroplasticity effects, brain regeneration, or any other focal or dispersed cognitive or behavioral function etc. such as for instance the application of an electromagnetic/electrical pulse/s following or preceding the presentation of specifically designated cognitive or behavioral stimulus/stimuli specifically geared to activate, train, re-train or modulate the neuronal, cognitive function or activation of a specific region, loci, locus in such a manner that the presentation of cognitive or behavioral or electromagnetic/electrical pulse/s can be synchronized before- after or during, simultaneously with the elicitation of certain neuronal activation or processing [e.g., that can all be synchronized and measured and controlled through the real-time measurement of the neuronal activity which appears prior to-following-or during the electromagnetic stimulation, cognitive or behavioral stimulus presentation (aimed at activation of specific diseased brain region/s).

The Brain Medic Stimulator system can also consist of stable or dynamic (mobile or stationary) electrical or electromagnetic stimulator/s capable of stimulating any cortical or sub-cortical brain region/s locus, loci etc. (such as through a helmet or implanted electrode and/or surrounding cortical or intra-cranial or orifices stimulators—that are interspersed and/or may even be interchangeable and/or conjointly or alternatively placed with the electromagnetic sensing device (or may even consist of the same electromagnetic or electrical chips, magnets, wires, coils etc.—capable of both producing electromagnetic/electrical stimulation and also of sensing or measuring electrophysiological activity in the brain such as ERP, EEG etc.); This dual and/or combined or interspersed electro/magnetic stimulation and electrophysiological measurement capacity of the BRAIN-MEDIC stimulator is also combined with its cognitive or behavioral stimulation or training or retraining capabilities to selectively stimulate specific brain region/s, functions, loci/locus, that may be convergent and/or synchronized and/or stimulate (e.g., in a synchronized manner—before, during, or after the electromagnetic stimulation and/or at various spatial brain region/s and/or different time-points in each of these regions or in a specific group of regions which may change or alternate/adjust across time).

A method or system or medical device or component/s of neurological activation or enhancement or modification of functions is described wherein a single and/or multiple implantable or sub dermal and/or subcutaneous electrode/s or chip/s and/or electrical and/or electromagnetic wire and/or coil and/or stimulating elements and/or in specific diseased brain region/s and/or which can be disease-specific and/or individual-specific etc. and/or which can be coupled together with sensing and/or electrophysiological sensing element/s and/or measurement of baseline and/or post-stimulation levels of: electrophysiological levels and/or neuroimaging and/or cognitive or behavioral functions, and/or which can also be aligned and/or work in coordination with adjustment or feedback and/or a specific computational module capable of adjusting and/or modulating and/or controlling the single and/or multiple electrode/s and/or stimulating element/s etc. based on either a pre-established norm and/or for healthy neuronal standards and/or functioning and/or for improvement or enhancement relative to disease-specific and/or individual-specific norms and/or such neuronal modulation can be targeted at reaching specific pre-determined healthy and/or performance and/or improvement value/s or norms and/or can be adjusted continuously (on-line or off-line) and/or until a certain improvement/s and/or a particular neuronal and/or functional level is attained. Finally, this method and/or system and/or medical device implantation can also be combined with external cognitive and/or behavioral stimulation modalities (such as goggles, computer screen stimulation and/or auditory, visual, olfactory or other sensory stimulation) which may be synchronized with the electrical and/or electromagnetic implantable or sub dermal stimulation protocol and/or regimen in order to enhance the positive therapeutic neuroplasticity effects and/or produce long-term lasting neuroplasticity effects.

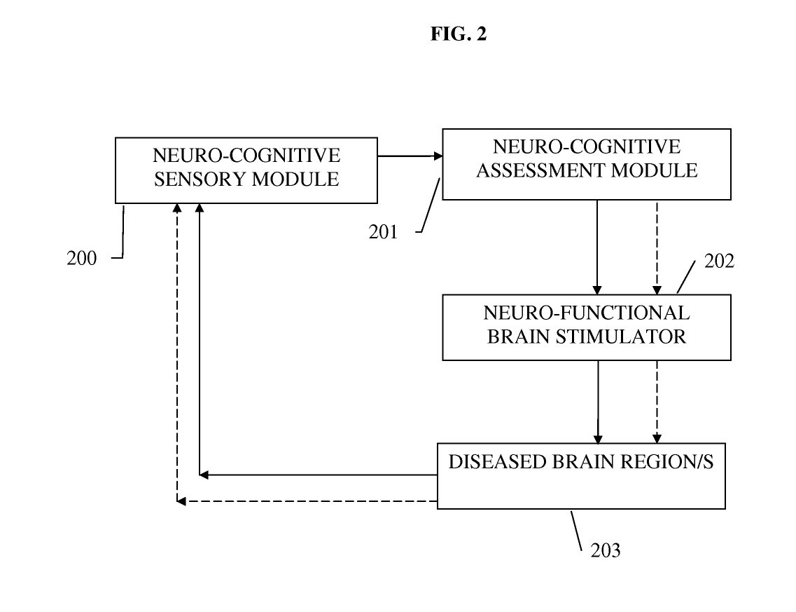

Reference is now made to FIG. 2 which describes a NEURO-COGNITIVE SENSORY MODULE 201 which is capable of measuring and/or assessing and/or indicating and/or at single or multiple time-points and/or for single and/or multiple brain DISEASED BRAIN REGION/S 203 region/s loci/locus various electrophysiological and/or EEG and/or ERP etc. and/or cognitive and/or behavioral measurement/s and/or at different spatial and/or temporal point/s and/or at baseline (and/or prior to any electrical and/or electromagnetic and/or cognitive and/or behavioral stimulation) and/or during- and/or following- and/or intermittently- with any electrical and/or electromagnetic and/or cognitive and/or behavioral stimulation; The NEURO-COGNITIVE SENSORY MODULE 201 outputs its electrophysiological etc. measurements to a NEURO-COGNITIVE ASSESSMENT MODULE 201 which is capable of comparing and/or contrasting and/or assessing and/or computing etc. the level/s and/or value/s of the measured (single or multiple spatial and/or temporal electrophysiological etc. value/s) found in an individual/s and/or diseased clinical group and/or in an individual and/or group with enhanced cognitive function/s—with predetermined and/or identified and/or measured norms of a normal population or sample and/or particular diseased sample or population and/or with norms or target functional performance norm/s, in order to determine the measured individual's and/or group's electrophysiological, cognitive level/s and/or behavioral etc. value/s relative to any of the abovementioned norms; Based on this analysis the NEURO-COGNITIVE ASSESSMENT MODULE 201 also determines the particular level/s and/or duration/s and/or intensity of electrical and/or electromagnetic stimulation and/or loci and/or locus of such single and/or multiple regions stimulation and/or synchronized or unsynchronized cognitive and/or behavioral stimulation that is necessary in order to modulate and/or improve performance and/or therapeutically affect positive neuroplasticity and/or cognitive and/or behavioral change in the individual's (or clinical diseased group's) state, and/or performance and/or brain activity value/s and/or levels; Specifically, the NEURO-COGNITIVE ASSESSMENT MODULE 201 is capable of adjusting the stimulation parameters (e.g., including but not limited to: brain single or multiple loci/locus of stimulation and/or intensity and/or duration and/or frequency of stimulation and/or convergent and/or synchronized/unsynchronized cognitive and/or behavioral stimulation) in such a manner which allows for the positive/enhanced/improved modulation of the relevant neuronal and/or behavioral and/or cognitive performance level/s and/or according to a predetermined and/or standards or norms of healthy and/or improvement relative to clinically diseased norms (e.g., pertaining to a particular brain disease).

The NEURO-COGNITIVE ASSESSMENT MODULE 201 then transmits or outputs its direction or control of a NEURO-FUNCTIONAL BRAIN STIMULATOR 202 in such a manner as to direct the localization and/or stimulation and/or electromagnetic and/or electrical and/or cognitive and/or behavioral stimulation parameters and/or intensity, duration, location, loci/locus etc. of the diseased brain region/s and/or specific to a particular disease/s and/or individual etc. and/or which are aimed towards achieving the predetermined and/or targeted brain functioning level/s and/or cognitive and/or behavioral and/or electrophysiological level and/or criteria etc.; The NEURO-FUNCTIONAL BRAIN STIMULATOR 202 stimulates therefore the (earlier identified) DISEASED BRAIN REGION/S 203 (e.g. at single and/or multiple spatial and/or temporal point/s) which then leads to a post- and/or intermittent- and/or synchronized-repeated measurement/s by the NEUROCOGNITIVE SENSORY MODULE 200 which is then inputted (once again) into the NEURO-COGNITIVE ASSESSMENT MODULE 201 that is capable of comparing the pre- and post-stimulation electrophysiological and/or cognitive and/or behavioral and/or performance and/or brain functioning level/s and also compare these pre- and/or post-stimulation brain/electrophysiological/cognitive/behavioral measurements with the earlier identified norms of healthy functioning/electrophysiology etc. or abovementioned norms and/or targeted improvement relative to diseased and/or healthy norms.

Hence, the NEURO-COGNITIVE ASSESSMENT MODULE 201 is capable of continuously monitoring and adjusting the NEURO-FUNCTIONAL BRAIN STIMULATOR 202's stimulation parameters (described above) in such a manner as to ensure and/or optimize and/or continuously and/or intermittently improve the neuronal activation and/or functional level and/or cognitive and/or behavioral level of performance until this feedback loop results in a measurement by the NEURO-COGNITIVE ASSESSMENT MODULE 201 of targeted NEURO-COGNITIVE ASSESSMENT MODULE 200 improved neuronal and/or cognitive and/or behavioral etc. brain functioning which can eventually lead to halting of the NEURO-FUNCTIONAL BRAIN STIMULATOR 202's (continuous or intermittent) NEURO-FUNCTIONAL BRAIN STIMULATOR 202's single and/or multiple spatial and/or temporal) stimulation of the DISEASED BRAIN REGION/S 203 (and/or once the standard norm/s have been attained).

The optimization of the NEURO-FUNCTIONAL BRAIN STIMULATOR 202's operation and function which is controlled by the NEURO-COGNITIVE ASSESSMENT MODULE 201 can be modulated in order to attain optimal criteria-based or performance based or relative to established health or enhanced functioning criteria which are determined by the NEURO-COGNITIVE ASSESSMENT MODULE 201 through several different ways which may include:

Comparison of normal neuronal functioning electrophysiological or electrical or neuroimaging based norms (performed within the NEURO-COGNITIVE ASSESSMENT MODULE 201) to diseased neuronal states or values and/or of the particular individual and/or of a diseased clinical group and/or to which the individual may be compared or belong to; Such comparison can be made for different states such as (for example) ‘resting’ state or ‘baseline’ and/or activation state/s and/or response to particular electrical and/or electromagnetic and/or cognitive or behavioral stimulus or stimuli etc.; Thus, one possible way of optimizing the functioning of the DISEASED BRAIN REGIONS 203 so as to fit and/or get closer to the specified or identified health norms determined by the NEURO-COGNITIVE ASSESSMENT MODULE 201 may consist of a comparison of the neuronal and/or cognitive and/or behavioral and/or neuroimaging based level and/or value/s of the afflicted and/or diseased and/or to be enhanced individual and/or clinical population group and/or diseased patients (and/or to which the individual belongs)—to a group of healthy individuals or norms of healthy functioning etc. in terms of baseline electrophysiological and/or cognitive and/or behavioral and/or neuroimaging or electrical measurement/s;

Such single or multiple spatial and/or temporal measurement/s or comparison/s between the individual (e.g., diseased or impaired or to be enhanced) and the desired norm of enhanced or normal or improved or even diseased brain states to be steered away from etc. can be based on the NEURO-COGNITIVE SENSORY MODULE's 200 concurrent, and/or single and/or multiple spatial and/or temporal measurement/s which can occur at the same and/or different spatial and/or temporal points as the stimulation by the NEURO-FUNCTIONAL STIMULATOR 202 such that it may allow to measure the response of electrical or electromagnetic stimulation occurring at the same or different spatial and/or temporal point/s as the NEURO-FUNCTIONAL STIMULATOR 202 which in turn can be compared across the normal vs. diseased population/s or individual/s (as compared by the NEURO-COGNITIVE ASSESSMENT MODULE 201). Thus for instance, one way of determining or modulating the operation of the NEURO-FUNCTIONAL BRAIN STIMULATOR 203 in order to optimize or improve or otherwise enhance neuronal brain functioning of the DISEASED BRAIN REGIONS 203 is through a comparison of either the response of the DISEASED BRAIN REGIONS 203 single or multiple neuron/s at single and/or multiple spatial and/or temporal points to electrical and/or electromagnetic and/or cognitive and/or behavioral stimulation (occurring and/or at single or multiple spatial and/or temporal points) and/or comparison of the DISEASED BRAIN REGIONS 203 functioning at baseline etc. between h normal and diseased individual/s or group/s.

The comparison of the diseased vs. normal or enhance and/or improved neuronal functioning can be based upon different localization/s and/or methodologies for measuring the NEURO-COGNITIVE SENSORY MODULE 200 by placing ERP, EEG and/or electrophysiological and/or electrical and/or electrode and/or magnetic measuring apparatus/es or agent either at the scalp (non-invasive) and/or invasively implanting recording electrodes and/or chips and/or magnetic coils and/or any other electrical or electromagnetic and/or electrophysiological measuring device or element/place at single and/or multiple spatial and/or temporal point/s; Thus, for instance, the optimization of the DISEASED BRAIN REGIONS 203 can be achieved through a comparison of EEG or ERP or any other non-invasive and/or invasive electrophysiological or electrical etc. measuring NEURO-COGNITIVE SENSORY MODULE 200 which compares normal or targeted neuronal functioning level—say at the scalp measurement with the diseased or impaired individual/s or clinical group, which yields a ‘gap analysis’ (carried out by the NEURO-COGNITIVE ASSESSMENT MODULE 201) of the necessary stimulation parameters to attain a closing of this gap in functional activation and/or cognitive and/or behavioral performance etc.; it should be noted that since it may be less feasible and/or desirable to implant such electrophysiological and/or electrical etc. NEURO-COGNITIVE SENSORY MODULE measuring (electrodes, chips, magnets, electrical wires etc.) within the brain of a normal individual—one way of being able measure and compute the existence of such ‘functional gap’ is through correlating or computing the relationship between ERP/EEG/electrophysiological activation and/or patterns measured at the scalp level and/or non-invasively and corresponding measurement/s at the invasive neuronal level of the DISEASED BRAIN REGION/S 203 locus—which would allow to extrapolate from the patient/s invasive electrophysiological and/or electrical etc. measurement/s to the normal control's or normal brain functioning norms attained or measured from the scalp measurements.

Based on such functional ‘gap analysis’ performed at the NEURO-COGNITIVE ASSESSMENT MODULE 201—e.g., relating to baseline, resting and/or activation and/or in response to any single or multiple electrical and/or electromagnetic and/or cognitive and/or behavioral and/or other stimulation and/or stimuli across the normal and/or targeted norms of functional activation or normal and/or enhance brain functioning norm/s and the single or group diseased or impaired neuronal and/or cognitive and/or behavioral and/or brain functioning and/or characteristic/s, the NEURO-COGNITIVE ASSESSMENT MODULE 201 determines what particular brain stimulation protocol and/or intensity and/or treatment plan (i.e., at single and/or multiple spatial and/or temporal point/s) should be carried out in order to attain the established and/or determined and/or computed health or norms and/or targeted norms for enhanced or less diseased desired states etc. Such determination of the optimal NEURO-FUNCTIONAL BRAIN STIMULATOR 202 stimulation protocol, intensity, regimen etc. can be determined or derived either through trial and error and/or experimentation and/or in real-time and/or off-line manners—based on the claims of this patent such as for instance through the introduction of single and/or multiple electrical and/or electromagnetic and/or cognitive and/or behavioral stimulations (at single and/or multiple spatial and/or temporal point/s) in facilitation or excitation of enhancement format/s as opposed to inhibitory format/s—if the desired objective is to enhance or facilitate the functioning of the particular hypo-activated or impaired or otherwise deficient neuronal functioning. ‘On-line’ of ‘off-line’ experimentation or computation and/or information derived from the literature all utilized in order to support one of the important claims of this patent application—e.g., based on the ability to compare and/or compute any modulation or improvement or enhancement and/or potentially positive therapeutic effect/s in the functioning (e.g. electrophysiological and/or electrical and/or electromagnetic and/or cognitive and/or behavioral etc.) of single and/or multiple neuron/s etc. through the application of appropriate facilitating or inhibiting electrical or electromagnetic and/or behavioral and/or cognitive stimulation to the appropriate neuron/s or brain region it should be possible to bring about a facilitation of an enhancement and/or alteration in the relevant neuron/s electrical or electrophysiological or electromagnetic or otherwise neuronal or neuroimaging based measurement of neuronal or brain activation (and by extension also possibly brain structure and general functioning);

As a matter of fact, a methodology and/or medical device and/or functional unit operation is hereby claimed wherein the NEURO-FUNCTIONAL BRAIN STIMULATOR's 202 optimization of the selected and/or identified DISEASED BRAIN REGION/S 203 measured electrophysiological or other related neuronal functioning can consist of an on-line or off-line continuous or disjoint adjustment and/or modulation loop which can consist of of-line pre-determined stimulation protocol, intensity, duration and/or any other stimulation parameters (e.g., at single and/or multiple spatial and/or temporal neuronal and/or brain loci/locus point/s) and/or cognitive and/or behavioral stimulation protocol/s and/or specifically in order to improve the neuronal and/or electrophysiological and/or electrical etc. and/or cognitive and/or behavioral functioning of a particular brain function, region/s, neuron/s and/or neuronal system- and/or which can also be based in part or whole on a particular individual's baseline and/or activation and/or in response to specific stimuli (e.g. broadly defined- including but not limited to electrical and/or cognitive and/or behavioral etc.); Such pre-determined stimulation protocols (as described above) can for instance be derived through investigation or known literature and/or computational algorithms claimed herein which are able to compute reliable relationship/s and/or associations between the neuronal ‘functional gap’ (described above—i.e., between the diseased and/or impaired individual/s and/or clinical group/s and the standard health norm and/or targeted neuronal functioning and/or cognitive and/or behavioral enhanced functioning etc., including also information regarding the values of the diseases individual/s or clinical group/s and the desired health and/or targeted standard etc.) and the neuronal stimulation paradigm and/or protocol and/or cognitive and/or behavioral stimulation and/or intensity, and/or frequency, and/or interval and/or otherwise all stimulation types and characterization/s—that are associated or and/or necessary and/or causing and/or bringing about a certain segment and/or aspect and/or the entire closing of the functional gap described above. In other words, the NEURO-COGNITIVE ASSESSMENT MODULE 201 is claimed to be capable of performing specific types of computational or algorithmic and/or decision-making steps and/or with or without assistance from a physician or expert in the field which can rely on pre-existing empirical knowledge or information and/or derive experimentation (off-line) in order to determine and/or compute the necessary stimulation parameters (broadly defined above) that are necessary or are associated and/or may bring about a specific value and/or magnitude of change in neuronal functional activity and/or pattern/s etc. required to close or diminish or positively affect the functional neuronal gap described above; And/or alternatively such off-line NEURO-COGNITIVE ASSESSMENT MODULE 201 can also be combined or replaced with an on-line continuous or disjoint NEURO-COGNITIVE ASSESSMENT MODULE 201 adjustment of the abovementioned stimulation parameters such that it can dynamically alter the NEURO-FUNCTIONAL BRAIN STIMULATOR's 202 stimulation parameters (e.g., including but not necessarily limited to electrical NEURO-COGNITIVE and/or electromagnetic NEURO and/or cognitive and/or behavioral and/or any other neuronal stimulation modality and/or element) based on on-going on-line dynamic computation of the targeted neuronal functioning level and/or closing of the neuronal functional gap relative and/or in comparison to the ‘current’ e.g. at time ‘ti1’ and/or ‘tin’ and/or spatial point/s ‘si1’ and/or ‘sin’—relative to the desired level and/or value of the neuronal functional level and/or the closing of the functional neuronal gap etc.; Such dynamic on-going and/or continuous and/or disjoint adjustment of the NEURO-FUNCTIONAL BRAIN STIMULATOR's 202 stimulation parameters can be also be based on computational and/or algorithmic rules and/or principles and/or formula's and/or in conjunction with real-time analytical computational determination of the most likely and/or appropriate stimulation parameters at a given point/in space and/or time;

Thus, it is foreseeable that a patient and/or diseased or impaired individual and/or physician and/or technician or other expert will be able to assist in such on-line real-time adjustment of the stimulation parameters—especially when/if the particular functions of this neuron/s can be phenomenologically and/or perceptually and/or empirically tested and/or compared to the normal or desired functional activation and/or pattern level and/or closing and/or diminishing of the neuronal gap; One classical example would be the continuous on-line controlling of patient's pain or epileptic event/s and/or motor dysfunction in Parkinson's and/or autistic children's degree of social alienation or fear or language performance level (also specifically relevant to aphasic patients etc.) or depressed patients mood level etc. Such self- and/or professional- and/or computational-(automatic and/or semi-automatic) monitoring of neuronal and/or corresponding cognitive and/or behavioral and/or motional and/or social etc. functioning correlates can be extremely powerful and useful in establishing long-term and/or short-term efficacy and/or optimization and/or clinical improvement new standard of care—in that they allow for fine-tuning and accurate determination of the precise neuronal stimulation protocol and/or intensity and/or frequency (see above broadly defined) that are necessary to improve and/or heal and/or close and/or improve or decrease the functional gap (differentiating between normal behavior and brain functioning and diseased or impaired individuals, clinical group/s or brain functioning).

In terms of the actual placement and/or possible mechanical design/s of the invented brain stimulation system described herein several different embodiments are possible including but not limited to: implantation of single and/or multiple brain stimulation elements (e.g. such as electrodes, coil/s electrical wires, chips etc. electromagnetic stimulating elements etc.) that can constitute a part of the NEURO-FUNCTIONAL BRAIN STIMULATOR 202 which can be placed at the same or different loci and/or locus and/or spatial configuration and/or spatial and/or temporal point/s as those of the NEURO-COGNITIVE SENSORY MODULE's 200 constitution (e.g., in part or in whole) of same and/or different spatial and/or temporal point/s and/or localization and/or same or different than the actual stimulating elements abovementioned as the NEURO-FUNCTIONAL BRAIN STIMULATOR 202's electrodes, coils etc. (e.g., it is foreseeable the two different configuration/s of the invention may consist of the same electrodes and/or coils etc. for both the NEURO-COGNITIVE SENSORY MODULE 200 and the NEURO-FUNCTIONAL BRAIN STIMULATOR 202—and/or parts of either of these elements may be cooperatively or interactively configured; and/or it may be the case that these two elements are entirely (and/or partially) separated but that they may occupy the same and/or different spatial and/or temporal point/s (note that one possible configuration of these elements may involve a dynamic mobilization of some of the NEURO-FUNCTIONAL BRAIN STIMULATOR 202 and/or the NEURO-COGNITIVE SENSORY MODULE 200). Hence, it is foreseeable that the invention may consist of same and/or different mechanical and/or electrical and/or computational and/or other elements shared or overlapping or closely interacting between the NEURO-COGNITIVE SENSORY MODULE 200 and the NEURO-FUNCTIONAL STIMULATOR 202—either in actual physical and/or computational and/or other parts and/or elements and/or configuration and/or independent (e.g., partially or wholly) but occupying the same spatial and/or temporal point/s and closely interacting and contributing to the abovementioned capacity to close the functional neuronal gap that may exist for diseased or impaired functions or individuals or clinical groups.

In terms of the actual operation of the brain stimulator described herein in this invention, there are a few possible formats or principles of operation or configurations, operationalization or structure of this medical device and/or methodology and/or elements which can include:
a. An initial pre-determined and/or interactive on-line implantation of the NEURO-COGNITIVE SENSORY MODULE's 200 elements and/or of the NEURO-FUNCTIONAL BRAIN STIMULATOR 202 (along with the proper configuration of the other elements of the invention, i.e., the NEURO-COGNITIVE ASSESSMENT MODULE 201 and all other elements), which is aimed at precise spatial configuration and/or implantation of the stimulating NEURO-FUNCTIONAL BRAIN STIMULATOR 202 elements at the most effective and/or efficacious and/or therapeutic and/or sensory effective spatial localization/s; Additionally, this phase is aimed at optimizing the initial level of stimulation for each of the NEURO-FUNCTIONAL BRAIN STIMULATOR 202 elements—according to either pre-determined protocols which may be individually tailored and/or determined per disease and/or cognitive and/or behavioral impairment/s and/or dynamically and/or on-line real-time adjustment of the stimulation parameters as described above) such that it allows for optimization of the neuronal functioning (or optimal stimulation parameters most likely to close and/or positively affect the neuronal functional gap) at the initial set up and/or implantation phase.
b. Longer term and/or on-going adjustment of the stimulation parameters (as described above) based on a continuous and/or disjoint measurements by the NEURO-COGNITIVE SENSORY MODULE 200 which is processed and/or computed by the NEURO-COGNITIVE ASSESSMENT MODULE 201 in order to determine the optimal stimulation parameters and/or any changes in these parameters necessary to positively affect the closing (and/or diminution) of the neuronal functional gap described above; Such NEURO-COGNITIVE SENSORY MODULE 200 measurements can be performed by the implanted electrodes or sensing elements periodically and/or continuously and/or with or without connection to the addition of intermittent cognitive and/or behavioral stimulation interval/s and/or protocols which can occur at different times and/or intervals and/or intensity and/or paradigms (as described above); Thus, for instance one possible embodiment of the device or invention herein described entails a continuous automatic and/or semi-automatic and/or physician and/or expert and/or central service and/or computation aided (expert) center NEURO-COGNITIVE ASSESSMENT MODULE 201 (which receives this continuous or intermittent input from the NEURO-COGNITIVE SENSORY MODULE 200 and automatically/semi-automatically etc. can adjust the stimulation parameters for that patient or individual (e.g., either through periodic or continuous transmission of controlling commands or input to the implantable elements of the NEURO-FUNCTIONAL BRAIN STIMULATOR 202) in order to optimize the stimulation parameters aimed at closing the neuronal functional gap; One embodiment of this invention is entirely automatic—with or without input from physician or expert-system input wherein the implantable NEURO-COGNITIVE SENSORY MODULE 200 along with the implantable NEURO-COGNITIVE ASSESSMENT MODULE 201 and the NEURO-FUNCTIONAL BRAIN STIMULATOR 202—which together form an adjustable real-time on-line loop enabling the automatic (or semi-automatic) adjustment of neuronal stimulation parameters to optimize or decrease the functional neuronal gap described above (and/or based on pre-determined rules, algorithms or decision rules and/or can also consist of a smart AI component which allows the system to continuously refine its dynamic stimulation parameters and may even learn or refine new stimulation rules constrained by safety and possibly physician aided and/or monitored follow-up); And/or this loop can be accessible for external physician-aided or expert based decision points or procedures which allow them to adjust, monitor, analyze and if necessary alter the stimulation parameters necessary to attain optimal neuronal functioning or closing/diminishing of the neuronal gap etc.; Note that it is also claimed that as part of the optimization of the NEURO-FUNCTIONAL BRAIN STIMULATOR 202 stimulation parameters geared towards closing or diminishing the functional neuronal gap it is claimed that intermittent or continuous on-line or off-line neurophysiological measurements (such as EEG ERP) and/or structural and/or functional neuroimaging measurements may assist in determining and/or adjusting the neuronal stimulation parameters.
c. On-going long-term stimulation parameters may be personalized and/or individual-based in order to optimize neuronal functioning or decrease the functional neuronal gap etc. and/or may consist of either neuronal stimulation (intermittent, on-going, continuous and/or single and/or multiple daily or weekly or recurrent intervals during allotted fixed time intervals or adjustable according to neuronal functioning)—which can be given separately and/or in conjunction with cognitive and/or behavioral stimulation—synchronized or unsynchronized etc.; This implies that one possible configuration of the invention may consist of an implantable electrical or electromagnetic etc. NEURO-FUNCTIONAL BRAIN STIMULATOR 202 elements which may operate on a continuous or disjoint and/or intermittent manner and/or may be personalized and/or optimized automatically or semi-automatically and/or through physician and/or expert based assistance—which may then also be combined with certain intervals (daily, weekly, hourly etc.) at which corresponding cognitive and/or behavioral stimulation is given (synchronized and/or non-synchronized) that stimulate the same and/or other neuron/s to produce neuroplasticity effects; Such convergent and/or combined and/or synchronized/non-synchronized cognitive and/or behavioral stimulation together with the neuronal stimulation (described above) can be coordinated or controlled via a centralized computer or any closed circuitry (hardwire, WIFI, or electronic or radio-based and/or electromagnetic transmission which allows to synchronize between the electrical/electromagnetic stimulation and the cognitive and/or behavioral stimulation event/s and/or parameters etc.) in order to enhance the therapeutic neuroplasticity effect/s.
d. Advanced possible embodiments of the invention also can call for precise time- and/or spatial-synchronization of the electrical and/or electromagnetic stimulation parameters of specific neuron/s of the DISEASED BRAIN REGION/S 203 together with the cognitive and/or behavioral and/or electrophysiological stimulation parameters or events such that the spatial and/or temporal synchronization and summation in fact enhances the neuronal functioning and the potential regenerative neuroplasticity effects;
e. As part and parcel of the synchronized or unsynchronized electrical and/or electromagnetic stimulation parameters which may be combined (or not) with the specific cognitive and/or behavioral stimulation parameter/s or event/s the invention also includes the possibility of providing sub-liminal or sub-psychophysical or sub-perceptual and/or perceptual and/or cognitive stimulation to facilitate neuronal functioning or closing of the functional neuronal gap.

A platform brain technology including real-time invasive or non-invasive adaptive neuro-behavioral ‘smart electrode’ comprising a method or system or medical device which allows for an interactive adjustment of invasive or non-invasive brain stimulation of diseased brain regions that are specific to particular diseases or to impaired brain functioning or for enhancement of intelligence or cognitive, behavioral or other brain functions in normal individuals.

The operation of the system is based on the stimulation of known (in the art or literature) diseased or mal-functioning diseased or impaired brain regions based on interactive measurements of neuronal and/or behavioral, cognitive or neurophysiological or other brain functioning index or indices and/or that may be specific to the disease being treated, and a ‘smart-electrode’ and/or or ‘smart-coil’ and/or ‘smart-chip’ and/or ‘smart computational module’ and/or which may be in response to preceding focal neuronal or behavioral, cognitive etc. stimulation and/or may be initiated without regard to such preceding neuronal or electric or electromagnetic invasive or non-invasive brain stimulation; The ‘smart’ stimulating element/s of the system (including the options or components described above) are capable of monitoring either in real-time or in fixed or flexible and/or continuous or disjoint times and/or cortical/subcortical locations the effects of the neuro-behavioral stimulation and/or neuronal and/or electrical and/or electromagnetic and/or any other brain stimulation and/or in single and/or multiple and/or cortical and/or sub-cortical etc. region/s or point/s, loci/locus, neuronal network/s or cell/s or functional element/s within the brain, and/or the smart component/s may monitor the abovementioned neuronal or brain features, activity, functioning, behavioral and/or cognitive and/or neurophysiological functioning and/or performance irrespective of the brain and/or neuronal etc. stimulation; Hence, the ‘smart chip’ or ‘smart electrode’ or ‘smart coil’ or ‘smart medical device’ and/or invasive and/or non-invasive is capable of adaptively altering the various parameters of stimulation (which may include but are not limited to: intensity, frequency, location/s at single and/or multiple and/or cortical and/or sub-cortical brain location/s and/or cell/s and/or functional organs within the brain and/or the entire brain etc.—in such a manner that it targets or aims at obtaining a specific normative or desired (healing and/or optimized and/or improved or enhanced and/or super-normal or above normal) functioning, behavioral or cognitive or neuronal or electrophysiological or electrical etc. state/s or levels of activation or performance etc. (e.g., which may be done with- or without regard to the interactive measurements of the neuron/s' response to stimulation, that is it could be done with or without regard to the responsiveness of the neuron/s or diseased or to be enhanced normal brain regions to stimulation); In any event the major point is that the smart component or medical device or electrode or coil etc. (also as described above) is capable of adjusting the various stimulation parameters (also as defined above) in order to attain a certain improved neuronal and/or functional and/or behavioral and/or cognitive state or improvement in disease parameters or brain's functioning or enhanced intelligence or brain functioning etc.—with or without regard to adjusting based on the measured neuron/s or brain or region/s response to stimulation.

In order to achieve this smart neuronal modulation objective or characteristic/s the system is able to adjust (in real-time or off-line) its stimulation parameters (described partially above) in several different important manners which include (but are not limited to):
1) The capacity to control, adjust or fine-tune or synchronize between the electrical and/or electromagnetic and/or neuronal and/or any other brain stimulation (e.g., and/or at single and/or multiple and/or brain region/s and/or loci and/or locus and/or cell/s neural network/s etc.) and any other behavioral or cognitive and/or other form of stimulation or manipulation (e.g., which may or may not additively contribute to the functioning or improvement or enhancement of the relevant or desired or targeted brain functioning or healing of disease or diseased or impaired functioning etc, or enhancement of intelligence or certain cognitive or behavioral functions etc.); This capacity to adjust or control or fine-tune the combination of neuronal and other cognitive or behavioral stimulation/s can be tied or not tied or linked with the real-time or off-line measurement/s of the neuronal functioning, electrical or electromagnetic or neuronal activity, values, parameters etc. such that the parameters of either the neuronal or electrical or electromagnetic and/or those of the behavioral or cognitive or other environmental manipulation will be adjusted to optimize the response or the activity or the independent functioning of the brain or diseased regions or individual for enhanced functioning or intelligence etc.
2) Hence, also a special consideration is given to the fine-tuning or controlling of the electrical stimulation or neuronal in order to obtain optimal or improved diseased region or diseased state improvement or enhancement of intelligence etc. A methodology is given wherein the relevant neuronal functioning is tested in ‘real-time’ via behavioral or neurophysiological or electrical (real-time or off-line measurement/s) which therefore inform the relevant neuronal stimulation parameters necessary to enhance or optimize or otherwise correct or improve or even heal the functioning of the desired or relevant neuron/s etc.

A Stimulus-Response Neuronal Diagnostic and modulation comprising of either a platform technology of a Stimulus-Response Disease-specific Neuronal Diagnostics and/or a platform technology of a Stimulus-Response Disease Specific Neuronal Modulation which may include several possible means for a system or a method or a medical device or a computerized aided diagnostic (physician determined or automatic) or assessment or evaluation or screening system which allows for a physician based or automatic assessment or diagnosis of disease-specific or generalized brain abnormality or abnormalities, or for determination of the likelihood of a specific brain disease or brain abnormality being present in an individual (e.g., including but not limited to the time of screening or diagnosis or prospectively at some other or future or follow-up testing or evaluation). This system is capable of identifying specific brain or neurological or neural or neuronal or cognitive or behavioral or neurophysiological abnormality or abnormalities or impairment or sub-normal values or performance of pattern of activity or condition or conditions or neuronal functioning etc. This screening or diagnostic or assessment system also allows for the identification of the specific brain or brain regional or region or regions or cell or cells or neuronal network or networks which are found to be abnormal or impaired or sub-normal or sub-normally functioning in an individual (e.g., including at different time-point/s), which also allows (among others) for the treatment or stimulation of these deficient or impaired or abnormal brain trait/s or cognitive or behavioral traits or regions or abnormalities or impairments to be stimulated including (but not limited to): neuronal stimulation, electrical, electromagnetic, electronic, neurophysiological, neuronal or cognitive or behavioral stimulation—including but not necessarily through some of the platform technologies or other technologies described in this application or applications. One of the principles of operation of this diagnostic platform technology is the measurement, comparison, or analysis or determination or algorithm or physician computerized aided diagnostic method or system or device which compares single or multiple disease-specific brain trait or traits including but not limited to neuronal, electrophysiological, behavioral or cognitive or other brain related traits or characteristics or values or patter or patterns or function or functioning or state/s or condition/s—to a standard of disease-specific criteria or levels or baseline or to health norm/s or criteria or functioning or brain trait/s etc. in order to determine whether or not the individual's brain condition/s or state/s or trait/s or functioning or brain region/s, neuron/s or cell/s or neuronal network/s are likely to be considered to be normal or healthy or are sub-normal or disease or belong to a disease-specific state or condition etc., which can then be utilized in order to provide for a probable or probability or likelihood that any specific or generalized or disease-specific or abnormal brain trait/s or characteristic/s is present (or may develop prospectively in the individual), or which specific abnormal or diseased brain trait/s or dysfunctional or sub-normal value or aspect/s or characteristic or brain region/s neuron/s is diseased or impaired etc. The diagnostic system can operate in ‘real-time’ or ‘off-line’ relative to the administration of stimulation (when the diagnostic system operates in conjunction or even as an integral part of the treatment or stimulation system). If or when the diagnostic system operates in conjunction or together with any neuro-cognitive or neuronal or electrical or electromagnetic or electronic or other stimulation components its operation can consist of an integrated system or method or medical device or components that can interactively adjust or continuously or dis-jointly adjust or control or modulate the operation or stimulation of the brain or specific brain region/s or cognitive or behavioral stimulation parameters (as also described elsewhere in this application/s). Thus, for instance one possible embodiment (best mode) of this invention can consist of (but not be limited to—or may be constructed in a different manner) to a system that includes implanted (or non-implanted or coil or electrical stimulation wires or stimulating elements) or implanted electrodes which may be implanted or placed at or near or stimulating the diseased brain region/s or cell/s or neuron/s or neuronal network/s etc. in such a manner that the selected (fixed, flexible, adjustable, controllable et.) stimulation parameters or the neuronal or electrical or behavioral stimulation provided at Ti . . . n (specific time point/s or interval/s or protocol/s) can be determined by the computation of the diagnostic system of the likelihood or probability of any specific brain trait/s or behavioral or cognitive trait or value or functioning etc. to be abnormal or normal or impaired etc.—which is then transferred to the computational module or component or algorithm of any brain stimulating or neuronal modulation system or device that can control the electrodes or stimulating elements or may even be an integral part of the stimulating electrodes etc. (with or without other cognitive or behavioral or other neuronal modifying or brain or cognitive or behavioral or electrophysiological modifying or treating elements) and may be adjustable or modified or changed or altered in order to provide sufficient or optimal or appropriate stimulation of the diseased brain trait/s or region/s or characteristic/s etc. that is likely or sufficient to bring about a sufficient or optimal or necessary change in diseased state or condition or impaired brain functioning. Therefore, the capacity of the diagnostic platform technology to provide pre-stimulation, or during—stimulation or post-stimulation or independent of stimulation measurement/s of the level of cognitive or behavioral or neurological or brain related functioning can be very useful in allowing for the determination of the likelihood of disease or brain disease specific or general or generalized or impaired or abnormal brain or cognitive or behavioral functioning and may also inform off-line or on-line, or fixed or adjustable neuro-cognitive or neuronal or cognitive or behavioral stimulation etc. which may therefore form an adjustable or fixed or closed loop or diagnostic or screening tool or device or methodology for diagnosing or assessing or treating any brain related disease.

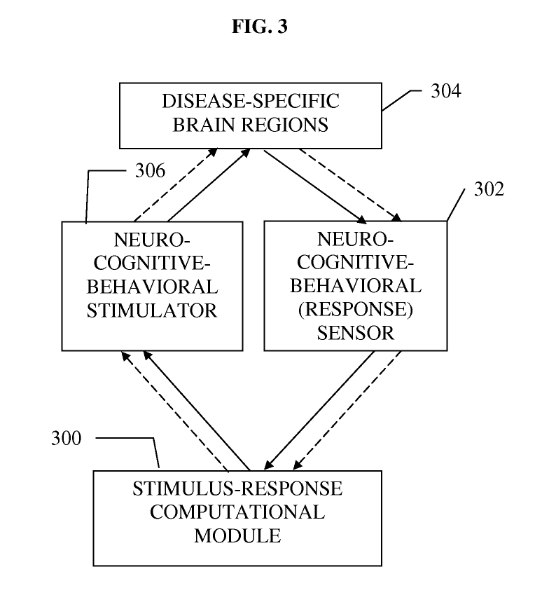

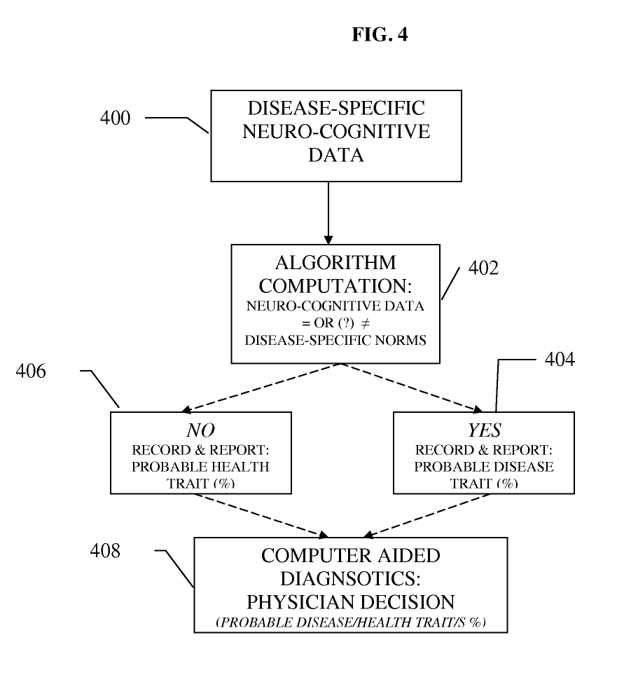

Reference is now made to FIG. 3 which describes a STIMULUS-RESPONSE COMPUTATIONAL MODULE 300 which can direct or control or output to the NEURO-COGNITIVE-BEHAVIORAL (RESPONSE) SENSOR 302 input with regards to which of the DISEASE-SPECIFIC BRAIN REGIONS 304 needs to be measured or sensed or analyzed (including electrophysiological, electromagnetic, activation or functional patterns, cognitive or behavioral or other neuronal measurements which may be taken at several different time point/s and/or spatial (cortical or subcortical or neuronal or cellular or neural-network), and/or the STIMULUS-RESPONSE COMPUTATIONAL MODULE 300 may also direct or control or output to the responsible for determining which can instruct or controls the a NEURO-COGNITIVE BEHAVIORAL STIMULATOR 306 which of the single or multiple DISEASE-SPECIFIC BRAIN REGIONS 304 needs to be stimulated including (but not limited to) electrically or electromagnetically or neuronally, or ultrasound or heat, light, or any other form of neuronal stimulation (which may occur or take place at different time point/s or interval/s or protocol/s etc.) It is important to note that either the NEURO-COGNITIVE-BEHAVIORAL (RESPONSE) SENSOR 302 can be activated or operated or made to measure specific single or multiple DISEASE-SPECIFIC BRAIN REGION/S 304—with or without connection or priming or preceding stimulation of the same or different DISEASE-SPECIFIC BRAIN REGION/S 304 by the NEURO-COGNITIVE BEHAVIORAL STIMULATOR 306. This is because one possible embodiment of the present invention can include the STIMULUS-RESPONSE COMPUTATIONAL MODULE 300's activation or direction/output to the NEURO-COGNITIVE-BEHAVIORAL (RESPONSE) SENSOR 302 to measure the neuronal or electrophysiological or cognitive or behavioral measures or values or indices or activity/activities of specific DISEASE-SPECIFIC BRAIN REGIONS 304—without concurrent or subsequent or previous s stimulation of the same or different DISEASE-SPECIFIC BRAIN REGION/S 304, or the activation or collection of data by the NEURO-COGNITIVE-BEHAVIORAL (RESPONSE) SENSOR 302 can be initiated or controlled or activated in synchronization with the STIMULUS-RESPONSE COMPUTATIONAL MODULE 300's stimulation (e.g., prior to- during- or after- neuro-cognitive or cognitive or behavioral stimulation) of the same or different single or multiple DISEASE-SPECIFIC BRAIN REGION/S 304. In the first case in which the NEURO-COGNITIVE-BEHAVIORAL (RESPONSE) SENSOR 302 collects neuro-cognitive or behavioral or cognitive data without previous or synchronized activation of the NEURO-COGNITIVE BEHAVIORAL STIMULATOR 306 or its stimulation of specific DISEASE-SPECIFIC BRAIN REGION/S 304, this type of measurement allows the system to obtain general or generalized or baseline measurements of neuronal or cognitive or behavioral measures in an individual relative to established health-norms or disease-norms or characterization/s; Such baseline or individual's ongoing neuronal functioning or disease state or characterization/s or cognitive or behavioral functioning can then be compared to established norms of disease or health or disease-specific characterization/s in order to determine whether or not an individual is exhibiting or is likely to be diagnosed or screened as suffering from a specific brain related disease or impairment or can also serve as potential prospective or predictive or probabilistic biomarkers or predictors or heuristics for the development of a particular brain related disease etc. (as also depicted in FIG. 4); Alternatively, the NEURO-COGNITIVE-BEHAVIORAL (RESPONSE) SENSOR 302 can operate or be activated by the STIMULUS-RESPONSE COMPUTATIONAL MODULE 300 in concurrence or in synchronization (including: before- during—or after- or in interval/s which can be activated along with) with the with the activation of the NEURO-COGNITIVE BEHAVIORAL STIMULATOR 306 which could allow for a measurement or assessment or diagnosis of neuronal or behavioral or cognitive functioning or characterization or diseased or healthy characterization or assessment based on the stimulus-response characterization of any DISEASE-SPECIFIC BRAIN REGION/S 304 response to electrical or electromagnetic or cognitive or behavioral stimulation (as also described in FIG. 2). Indeed, FIG. 4 describes some of the possible functions of the STIMULUS-RESPONSE COMPUTATIONAL MODULE 300 within the diagnostic system described above, which can also include: obtaining DISEASE-SPECIFIC NEURO-COGNITIVE DATA 400 which would be the input provided or given by the NEURO-COGNITIVE BEHAVIORAL (RESPONSE) SENSOR 402 which is inputted into the STIMULUS-RESPONSE COMPUTATIONAL MODULE 400 and can include neuronal, or neural or neural-network/s or neural- or neuronal-activation or state or condition (whether independently of any stimulation or as a response to certain electrical or electromagnetic or behavioral or cognitive or other stimulation or training or activation, or cognitive or behavioral or other response or baseline performance, value/s level/s etc. and may be measured at single or multiple spatial or temporal or spatial-temporal point/s or region/s or diseased brain region/s or cell/s, neural network/s etc. This DISEASE-SPECIFIC NEURO-COGNITIVE DATA 400 is then inputted into the ALGORITHM COMPUTATION 402 which can include such processing or algorithm or decision-rule based computation or analyses or determination including but not limited to: the comparison or analysis or processing of single or multiple NEURO-COGNITIVE DATA=OR (?)≠DISEASE-SPECIFIC NORMS, which essentially means that this processing can also compare any single or multiple or array or combination of the DISEASE-SPECIFIC NEURO-COGNITIVE DATA 400 as also described above to disease-specific norms for neuronal or neural or electrophysiological or cognitive or behavioral or other brain related impairment/s or condition/s or performance level/s or patterns of deficiency/cies etc. in order to determine whether the observed DISEASE-SPECIFIC NEURO-COGNITIVE DATA 400 fits or belongs or can be statistically or probabilistically associated with an impaired or dysfunctional or disease or diseases of the brain or specific brain, cognitive or behavioral disease, impairment or sub-normal condition, state, functioning, or performance etc. Hence, the ALGORITHM COMPUTATION 402 performs an analysis or computation which allows for the determination of the likelihood of a specific brain disease or brain impairment of subnormal condition or state to be present (including at the current time of measurement or as predictive of the likelihood of developing or manifesting certain clinical disease/s or pre-clinical or pre-symptomatic disease/s or abnormal condition/s or malfunctioning etc.). Hence, based on the ALGORITHM COMPUTATION 402 analysis r computation or decision-rule or expert-based analysis also described above it outputs one of two possible outcomes or outputs or decisions or likelihood readings etc.: YES 404 which implies that the NEURO-COGNITIVE DATA=DISEASE SPECIFIC NORMS, implying or being outputted in cases in which the analysis of a certain single or multiple, integrated or independent component/s of the cognitive or behavioral or neuronal or electrophysiological or other brain related reading/s or measurement/s or correlates (which can be measured in single or multiple spatial- or temporal or spatial-temporal point/s) indicates that there is a certain likelihood of probability or association or linkage etc. between the individual's DISEASE-SPECIFIC NEURO-COGNITIVE DATA 400 or NEURO-COGNITIVE DATA the likelihood of exhibiting, possessing, or manifesting or prospectively being likely to develop or manifest a certain brain related disease or impairment/s or abnormal or subnormal condition etc. Therefore the ALGORITHM COMPUTATION 402 can also include single or multiple, simple or complex statistical, computational, probability-based, heuristic-based or expert-based mathematical or computational or algorithm principles or methodologies or means of determining or calculating or deriving the likelihood of a certain brain condition or specific brain disease or impairment/s or sub-normal functioning or abnormal performance or condition to exist (e.g., either at the time of measurement or prospectively at some other time or spatial region/s in the brain etc.), which may also involve the derivation of certain disease-specific or condition-specific or impairment-specific threshold/s (which may also take into account single or multiple factors such or predictors or biomarkers or algorithm-specific or computational formula cut-off values or threshold/s etc. that may be utilized to determine the likelihood of an individual/s possessing or being diagnosed or being afflicted or likely to develop a certain brain-related disease/s or condition/s etc.). In case the ALGORITHM COMPUTATION 402 outputted the YES 404 output or computational result then the single or multiple ‘t−0’, i.e., abnormal or disease-specific or condition-specific traits or cognitive or behavioral or electrophysiological trait/s measured at the time of the diagnosis or testing or as existing or being likely or being diagnosed at the time of testing or measurement/s or at ‘t−i’—i.e., as predictive or a likely prediction for the development of a certain clinical condition/s or brain disease/s or impairment/s etc. are being recorded in the system.

In case the ALGORITHM COMPUTATION 402 yields a NO 406 output or computational result this means that the comparison of the DISEASE-SPECIFIC NEURO-COGNITIVE DATA 400 or the NEURO-COGNITIVE DATA (in 402) with disease-specific norms or impaired condition/s or abnormal or sub-normal brain functioning or cognitive or behavioral performance or level/s or behavior etc. was found to not be likely associated or belonging or indicative (either at ‘t−0’ or at ‘t−i’ etc. or prospectively or probabilistically etc.) of any brain abnormal condition or disease/s or cognitive or behavioral deficiency or tendency towards abnormality or sub-normal or deficient or impaired performance etc.; Here too, the computational analysis or algorithm-based analysis or determination etc. can involve sophisticated or simple single or multiple computational methodologies or any mathematical or any regression or any statistical methodology or system or computerized or computational methodology capable of determining the likelihood of the individual (at ‘t−0’ or ‘t−i’) possessing or exhibiting or being likely to suffer or develop or have the chances of suffering or contracting a specific brain disease/s or condition/s or impairment/s etc.), which may also include the abovementioned cut-off or threshold or other statistical or mathematical or heuristic or expert-based or decision-rule/s or algorithm methodology of determining the likelihood or the cutoff point/s or value/s under- or above- or at- which the individual is deemed as likely or as possessing a certain likelihood to suffer, or being diagnosed or manifest or develop a certain brain disease or impairment or cognitive or behavioral or neuronal abnormality etc.

Some of the possible structure/s or configuration/s or best-mode/s embodiment/s of this system can allow for either the utilization of the YES 404 or NO 406 output/s or computational result/s etc. directly to be utilized for assessment of reporting or diagnosis or likely development or manifestation or diagnosis of a particular disease/s or brain illness/es or condition/s or abnormal condition or cognitive or behavioral or neurophysiological or electrophysiological state/s or condition/s or disease/s or impairment/s—including the possibility of reporting or outputting these result/s as probabilistic or ‘likelihood’ or ‘heuristic’ or ‘possible’ results or assessment/s or diagnosis or diagnostic/s etc. or as diagnosis or prospective likelihood of developing a disease or certain abnormal brain functioning etc.; Or these computational results YES 404 or NO 406 may be used or outputted to a COMPUTER AIDED DIAGNOSTICS: PHYSICIAN DECISION 408 which essentially allows the physician or a technician or any other authorized clinician or care-provider or professional to utilize or consult or consider or evaluate etc. the validity or clinical appropriateness or likelihood or usefulness of applicability or suitability or clinical relevance or diagnostic likelihood or relevance or diagnosis of the patient or individual being assessed or diagnosed or measured—with the ultimate decision or diagnosis being laid in the hands of the physician or technician or other professional or cognizing human being. In any event, the precise single or multiple DISEASE-SPECIFIC NEURO-COGNITIVE DATA 400 or NEURO-COGNITIVE DATA (in 402) neural or neuronal or cognitive or behavioral or electrophysiological or other brain related or impairment related data or aspect/s or value/s or specific impaired brain region/s neuron/s or neuronal network/s or particular cognitive or behavioral deficient or diseased or impaired trait/s or performance/s or value/s etc. will be specifically outputted at either the YES 404/NO 406 output of the system (which can be directly utilized—as for example in FIG. 4 or by any end-user that is deemed appropriate or necessary or qualified to receive such automated diagnostic-likelihood of disease/s data, or alternatively can be filtered an further processed and make a decision by a qualified physician or any other qualified or proper or appropriate professional which may integrate or rely or assess this single or multiple aspects or data contained within the DISEASE-SPECIFIC NEURO-COGNITIVE DATA 400.

Another important point or aspect of the diagnostic platform described herein is that there exist at least three different possible embodiments or best mode/s for one of the diagnostic aspects of this invention, including but not limited to: Disease-Specific Diagnostic System/s (DSDS) or a symptom- or deficiency- or ‘Condition-specific Diagnostic System (CSDS), or a generalized ‘Neuro-Cognitive Abnormality Index’ (NCAI), which may also lend itself to a computer aided diagnostic format; Hence, the DSDS, Disease Specific Diagnostic System is capable of diagnosing or assessing or screening or predicting or foreseeing or assessing the likelihood of a person or individual or patient manifesting or expressing or being afflicted by a particular brain disease or impairment or the likelihood of that individual developing this or that disease or brain condition at a prospective time point or time or interval in the future etc. This DSDS embodiment or best mode definition targets or aims at identifying specific brain abnormal conditions, diseases or symptoms of disease/s or illnesses which may be well defined and categorized clinically or medically based on the above illustrated and outlined system which compares the individual's DISEASE-SPECIFIC NEURO-COGNITIVE DATA 100 with particular DISEASE-SPECIFIC NORMS (in 402) which can include all of the specific neural or neuronal or diseased brain region or regions cortically or sub-cortically or cognitive or behavioral specific deficits or impaired functioning or performance etc. (which may be determined as explained above through particular ALGORITHM COMPUTATION 402 threshold/s or cut-off value/s as determined or computed through statistical or mathematical means etc. The goal and objective of the DSDS is hence the capacity to determine whether a particular individual suffers from a particular disease or diseases or (well-defined) impairment/s (at the time of diagnosis, ‘t−0’ or at a prospective time ‘t−i’)—which may imply that a YES 404 or NO 406 computational output (whether confirmed or processed by the COMPUTER AIDED DIAGNOSTICS: PHYSICIAN DECISION 408 or whether directly outputted for diagnostic or assessment or predictive or for treatment planning or interface with the platform technology neuronal modulation described below or any other brain treatment or neuro-cognitive of cognitive or behavioral or other brain related or disease-related intervention) implies or states or means or informs that the individual is either diagnosed or likely or screened or may possess or be inflicted etc. or develop in the future etc. particular brain disease/es or brain illness/es, or that the person is not afflicted or likely to be afflicted or to develop in the future any single or multiple brain disease or cognitive or behavioral disease/s etc. This means that the objective or end-result of the DSDS application or embodiment or best-mode of this diagnostic system allows this system to determine the likelihood or the presence or the existence of a particular brain-related disease or the likelihood of the development of such single or multiple brain disease or diseases or impairment/s etc.;

In contrast, the CSDS targets the identification or screening or diagnosis or assessment or screening of the likelihood of the person possessing or having or being afflicted with a particular abnormal or sub-normal brain condition/s or state, or impairment/s or reduction in particular cognitive or behavioral or electrophysiological functioning or performance/s or condition—which may be temporary or permanent or which may arise from different brain disease/s or environmental, or cognitive or behavioral or other factors or condition/s, including the possibility of different individuals or patients suffering from the same or different brain or cognitive or behavioral debilitating or impaired conditions or cognitive or behavioral or electrophysiological impairment/s—which may be due to different or the same underlying condition/s or disease/s or illness/es etc; Thus, for instance, dementia and alcohol or substance abuse may lead to same or similar or different cognitive or behavioral or neurophysiological or electrophysiological impairment/s or condition/s or abnormality/ties as can be measured by the CSDS (despite the fact that the underlying disease/s or state/s or condition/s in each of these patient/s may be different). Thus, the CSDS diagnostic application is unique or is characterized by the fact that it allows for the diagnosis or screening or assessment of the existence or the prospects or the likelihood of the existence or development of a particular brain condition/s or abnormality or sub-normal functioning or cognitive or behavioral state/s or deficiency/ies etc.—which may be temporary or fixed or dynamic or which may be caused or associated or be a part of different (underlying) brain disease/s or abnormalities or conditions or impairment/s. Finally, the ‘Neuro-Cognitive Abnormality Index’ (NCAI) adopts a different diagnostic implementation or approach or target/objective which is primarily geared towards differentiating or being able to diagnose or screen or differentiate or discern between normal vs. abnormal brain or cognitive or behavioral or electrophysiological value/s or performance/s or pattern/s or state/s etc.—also relying on the ALGORITHM COMPUTATION 402 capacity to determine or compute or analyze or process or compare between statistically or likely or probabilistic or decision rule/s' or cut-off or threshold/s computed differences between what normal value/s or pattern/s or region/s activation, electrophysiological activity or cognitive or behavioral or other brain performance or activity level or capacities etc.—either as baseline or in response to a certain stimulation/s etc.; Hence, the NCAI's output or determination of either the YES 404 or NO 406 computational or mathematical or decision or algorithm computed output or decision indicates or signifies that the individual is likely or probable or there is a certain likelihood or association or predictive strength to suspect or postulate to determine that an individual is either considered to be ‘normal’ in his or her brain or cognitive or behavioral function/s or functioning or capacity/ies or performance or neuronal functioning etc. or that that individual is likely or has a certain probability to exhibit or manifest or posses or develop any brain abnormality or disease etc.; Hence, the NCAI determines whether or not the person is deemed to have normal or abnormal brain parameters or performance or cognitive or behavioral or other brain related activity, capacity, capability or capabilities or performance etc. As such the NCAI may be utilized as a first generalized screening of overall brain capacity or predictive function as to the brain's overall normalcy or of the likelihood of the person suffering from any brain deficiency or abnormality or any disease (e.g., either at the present time ‘t−0’ or at any future or prospective other time point or interval ‘t−i’). which may be augmented or followed or supplemented with a secondary DSDS or CSDS analyses or computation/s etc. In fact, each of these different best-mode possible embodiment/s can be performed separately or in combination with any other possible embodiment/s

It should also be noted that a few possible best-mode embodiment/s of this invention can either combine or separate between the diagnostic and therapeutic element/s including for instance the capacity to treat various brain-related illnesses or condition/s or sub-normal condition/s or performance/s or disease/s—with or without reliance or involvement or interaction with the diagnostic element of the invention, i.e., for instance treating Autism or Parkinson's based on a uniform or fixed or adjustable neuro-cognitive or behavioral stimulation of known in the art diseased brain regions specific to each of these diseases such as for instance treating Autism through the (uniform) stimulation of autistic children (or low-functioning or non-verbal Autistic children) with electrical or electromagnetic or neuronal stimulation of the right hemisphere's contralateral Broca's and Wernicke's region/s, stimulating the cingulate gyrus (left or right), Amygdala or Fusiform Gyrus—with or without need for a preceding diagnostic application (e.g., either of the three or other possible embodiments of the platform diagnostic technology described above). Thus, it should be emphasized that while some sort of combination between the diagnostic platform technology and the therapeutic neuronal modulation platform technology may be desirable at certain instances and may offer a potentially robust fine-tuning and individual-based dynamic or fixed adjustment of the precise neuro-cognitive or behavioral or other neuronal or disease-specific stimulation which can also be based upon the diagnostic (synchronized or independent, single or multiple spatial-temporal diagnostics or assessment/s) it is not necessary to utilize a diagnostic modality in order to inform or direct or control the application of the neuronal modulation platform technology described in this patent application/s.

A platform technology of Stimulus-Response Disease-Specific Neuronal Modulation outlines several possible means for a system or a method or a medical device or implantable or electrode or electrodes or coil or coils that are capable of stimulating disease specific brain region or regions or cell or cells or neuronal network or networks or which can modulate neuronal activity or performance or level or levels or pattern or patterns in order to achieve a more normalized pattern of activation or improved cognitive or behavioral or neurophysiological activity or activation of state or condition (which can be compared to a normal state or normal activity or normal or optimal or enhanced neuronal activity). The determination of the required stimulation parameters (including but not limited to: electrical or electromagnetic or electronic or any other neuronal stimulation including heat, light, electromagnetic, sound etc., and/or cognitive or behavioral or sensory or mechanical etc.) that are necessary in order to produce or attain or achieve the desired or criteria-based normalized or optimized or improved relative to the diseased state or baseline or pre-stimulation state or condition can be determined based on a mechanism or on a best mode embodiment which can consist of a (disease-specific) neuronal stimulation module or element that stimulates the diseased single or multiple brain region or regions, cell or cells, neural-network etc. and then measure the response to this stimulation (by another module) and compares the stimulus-response data of an individual to the standardized norms of healthy functioning or norms or activity or activation or performance or cognitive or behavioral performance levels.

One possible embodiment of the PLATFORM TECHNOLOGY: STIMULUS-RESPONSE DISEASE-SPECIFIC NEURONAL MODULATION relies on the same description provided by FIG. 3—specifically it may involve the STIMULUS-RESPONSE COMPUTATIONAL MODULE 300 which is capable of determining and instructing the NEURO-COGNITIVE BEHAVIORAL STIMULATOR 306 which of the particular DISEASE-SPECIFIC BRAIN REGIONS 304 needs to be stimulated and in what precise stimulation parameter/s including but not limited to: electrical or electromagnetic or neuronal or neurophysiological or electrophysiological or cognitive or behavioral or motor or other bodily or physical or sensory manipulation or training or stimulation etc. or what frequency, duration, electrical or electromagnetic stimulation intensity, duration, interval, or what single or multiple cortical or sub-cortical brain region/s or cell/s or neuron/s or neuronal network/s etc.—i.e., with or without a preceding or synchronized or concurrent or independent activity or initiation of the NEURO-COGNITIVE BEHAVIORAL (RESPONSE) SENSOR 302 which may inform or screen or diagnose or compute the likelihood of an individual suffering or manifesting or provide a predictive evaluation of the likelihood of an individual developing a specific brain disease/s or impairment/s or abnormal functioning or performance/s etc. Hence, the STIMULUS-RESPONSE COMPUTATIONAL MODULE 300 is capable of instructing or controlling or modulating the NEURO-COGNITIVE BEHAVIORAL STIMULATOR 306 which of the DISEASE-SPECIFIC BRAIN REGIONS 304 to stimulate and in which manner, what stimulation parameters etc. to utilize—which may be fixed or adjusted or changing or dynamic and may also interact or rely or be fine-tuned based on a concurrent or a synchronized (i.e., before, during, or after or throughout the time of neural or neuronal or cognitive or behavioral or other neural stimulation) or which may measure neuronal or cognitive or behavioral level/s, performance/s, activity, functioning etc. stimulation independently of the stimulation parameters or time, location spatial or temporal or spatial-temporal localization etc. Hence, the operation of the NEURO-COGNITIVE-BEHAVIORAL STIMULATOR 306 can be intertwined or operated interactively or in combination with the some sort of functioning or activation of the NEURO-COGNITIVE BEHAVIORAL (RESPONSE) SENSOR 302 or may be operated independently based on the pre-determined or determined DISEASE-SPECIFIC BRAIN REGIONS 304 as determined by the STIMULUS-RESPONSE COMPUTATIONAL MODULE 300. Note also that the such ad-hoc or pre-determined selection of the precise DISEASE-SPECIFIC BRAIN REGIONS 304 to be stimulated (by the STIMULUS-RESPONSE COMPUTATIONAL MODULE 300) based on known in the art specification/s of the diseased brain region/s or cognitive or behavioral or functional or neuronal specific to each brain disease or condition also relates to any of the possible or potential brain impairment/s or abnormalities or sub-normal functioning or (temporary or long-term or intermittent) deficiency or deficiencies etc. (as also determined or computed by the diagnostic platform technology including its three specified diagnostic applications).

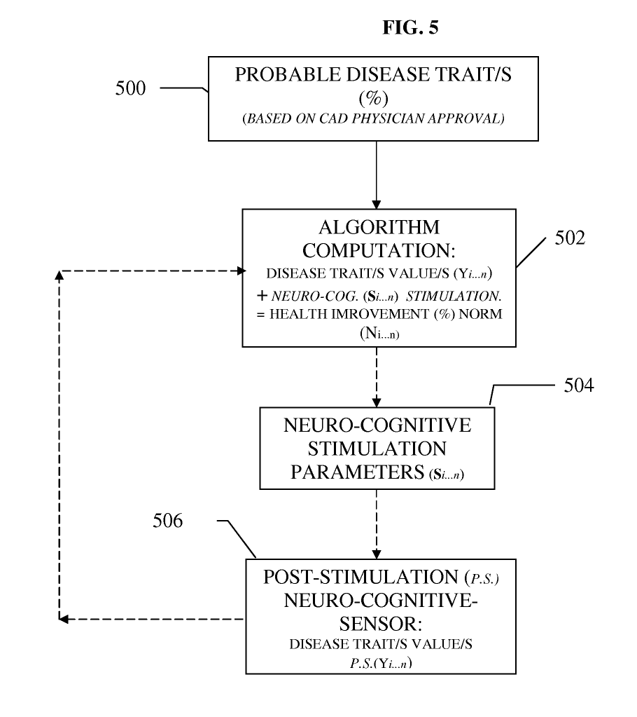

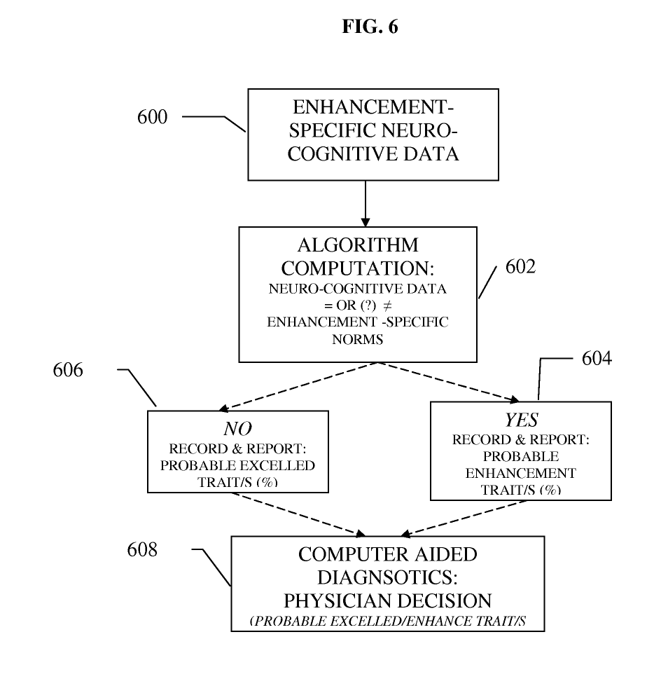

Reference is now made to FIG. 5 which can also describe some of the computational functions that may be carried out by the STIMULUS-RESPONSE COMPUTATIONAL MODULE 300; Hence, one of the possible computational functions carried out by the COMPUTATIONAL MODULE 300 (depicted in FIG. 3) includes the PROBABLE DISEASE TRAIT/S 500 which refers to the output of FIG. 4's YES 404 or NO 406 output or computational results, which essentially details or specifies either whether or not there is a likely diagnosis or screening or prospective likelihood or existence of a specific disease/s or abnormal brain condition/s or deficiencies or temporary or permanent or long-term or short term brain or cognitive or behavioral disability or impairment/s etc. or whether any specific brain region/s or cognitive or behavioral or neuronal or neural impairment of abnormality exist/s (which may underlie different or the same brain or mental or cognitive or behavioral disease/s or condition/s etc.)—with or without the assistance or reference or judgment of a physician or qualified professional etc. (through the COMPUTER AIDED DIAGNOSTICS: PHYSICIAN DECISION 408). Thus, the PROBABLE DISEASE TRAIT/S (%) 500 specifies either if the person has or is likely to have or may likely develop a particular brain disease/s or illness/es or abnormal or impaired condition or cognitive or behavioral functioning etc. or which of the individual's brain region/s neuron/s or cell/s or neuronal network/s or cognitive or behavioral or other brain related trait/s or aspect/s or functioning or state/s may be impaired or abnormal or deficient etc. or what's the likelihood of any such brain region/s or behavioral or cognitive trait/s being impaired or abnormal etc. Then, the output of the PROBABLE DISEASE TRAIT/S (%) 500 is outputted to the ALGORITHM COMPUTATION 502 which is capable of computing the necessary or desired NEURO-COG (Si . . . n) STIMULATION parameters necessary to attain or achieve a particular HEALTH IMPROVEMENT NORM (%) (Ni . . . n); Thus, the ALGORITHM COMPUTATION 502 is capable of determining for each of single or multiple PROBABLE DISEASE TRAIT/S 500 (of FIG. 5) including but not limited to any single or multiple neural or neuronal or cognitive or behavioral or brain region/s or cell/s or neuronal network/s etc. what is the necessary stimulation parameters including but not limited to: electrical or electromagnetic or electrophysiological or neuronal or cognitive or behavioral etc. stimulation or intensity, frequency, location, or spatial or temporal or spatial-temporal stimulation parameter/s or protocol, duration, length, interval adjustment, fine-tuning (including based on the neuronal of cognitive or behavioral response to stimulation etc., which will be further discussed below). One of the possible best-mode/s embodiment/s of this ALGORITHM COMPUTATION 502 operation or function is an embodiment in which this ALGORITHM COMPUTATION 502 can be carried out at the location or independently by each implanted electrode/s or non-invasive neuro-cognitive or behavioral stimulation of any single or multiple disease-specific or impairment-specific region/s—independently of any NEURO-COGNITIVE BEHAVIORAL (RESPONSE) SENSOR 302 or in conjunction with the operation of such NEURO-COGNITIVE BEHAVIORAL (RESPONSE) SENSOR 302. According to such best mode embodiment each electrode or (invasive or non-invasive) stimulating element can operate independently and contain or be provided or utilize a specific output or internal computation pertaining to its own neuro-cognitive or behavioral stimulation parameters such that the neuro-cognitive stimulation parameters of any particular electrode or coil or wire or region-specific or disease-specific stimulating element is guided or controlled by this NEURO-COGNITIVE BEHAVIORAL (RESPONSE) SENSOR 302 which may be an integral part of the particular stimulating element/s or of a particular disease/s or condition/s or specific brain region/s etc.; Or alternatively, the computation of the NEURO-COGNITIVE BEHAVIORAL (RESPONSE) SENSOR 302 can be carried out by a centralized or several different computational engines or module/s which may be fixed or adjustable or carried out ad-hoc prior to or independent of any neuro-cognitive or behavioral or electrophysiological stimulation or could be carried interactively or iteratively or synchronized with the result/s or response/s or measurement/s of the neuro-cognitive stimulation of any single or multiple brain region/s stimulation. Thus, it is conceivable as one of the possible best mode configuration/s of this invention to have ‘smart’ electrodes or ‘smart stimulating elements’ which are capable of stimulating disease-specific or condition-specific or impairment—specific brain region/s or cognitive or behavioral or electrophysiological trait/s or region/s or loci etc.—either based on ad-hoc or disease-specific or impairment-specific stimulation parameters or based on synchronized or initiated NEURO-COGNITIVE BEHAVIORAL (RESPONSE) SENSOR 302. Indeed, the ALGORITHM COMPUTATION 502 outputs its computational determination or output of the necessary or desired or needed NEURO-COGNITIVE STIMULATION 502 in order to obtain a certain likelihood of improvement in the PROBABLE DISEASE TRAIT/S 500—in the form or format of NEURO-COGNITIVE STIMULATION PARAMETERS 504 which are essentially fed into or outputted to the NEURO-COGNITIVE BEHAVIORAL STIMULATOR 306 which is responsible for the stimulation of the relevant or desired or needed brain region/s or cognitive or behavioral or other neuronal parameters. Following this stimulation (e.g., of single or multiple brain region/s or diseased or impaired or cognitive or behavioral or sub-normal or disease-specific or condition-specific stimulation/s) there is the possibility of adjusting the ongoing or intermittent or other POST-STIMULATION NEURO-COGNITIVE SENSOR 506 based on a repeated or modified ALGORITHM COMPUTATION 502 which again compares between the established health norms and the patient's exhibited post-stimulation disease-specific measured traits etc. This loop of neuronal or cognitive or behavioral or brain stimulation which is aimed at improving or healing or curing or optimizing the performance or improving the diseased state or disease specific or impairment specific dysfunction/s can be repeated until a certain health-norm is attained or a certain improvement or even curing or healing of the impairment or disease/s etc.

A platform technology for Stimulus-Response-Enhancement-Specific Neuronal Modulation outlines several possible means for a system or a method or a medical device or implantable or electrode or electrodes or coil or coils that are capable of stimulating in order to enhance or improve specific brain region or regions or cell or cells or neuronal network or networks or which can modulate neuronal activity or performance or level or levels or pattern or patterns in order to achieve a more normalized pattern of activation or improved cognitive or behavioral or neurophysiological activity or activation of state or condition (which can be compared to a normal state or normal activity or normal or optimal or enhanced neuronal activity). The determination of the required stimulation parameters (including but not limited to: electrical or electromagnetic or electronic or any other neuronal stimulation including heat, light, electromagnetic, sound etc., and/or cognitive or behavioral or sensory or mechanical etc.) that are necessary in order to produce or attain or achieve the desired or criteria-based enhancement or maximization relative to normal brain functioning or baseline or pre-stimulation state or condition can be determined based on a mechanism or on a best mode embodiment which can consist of a enhancement-specific neuronal stimulation module or element that stimulates the diseased single or multiple brain region or regions, cell or cells, neural-network etc. and then measure the response to this stimulation (by another module) and compares the stimulus-response data of an individual to the standardized norms of healthy functioning or norms or activity or activation or performance or cognitive or behavioral performance levels.

Essentially, this application would simulate or perform the same function/s and procedure/s as the applications for neural or cognitive or behavioral modulation described above—but for the purpose of enhancing cognitive or behavioral or neuronal or brain functions is normal individuals or in individuals in which there is a need or desire to enhance or optimize or improve their various function or functions.

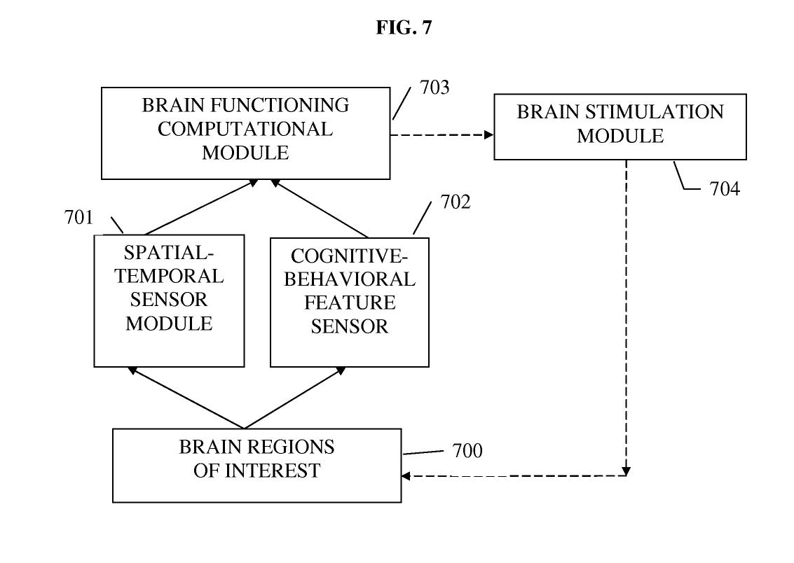

A system or method for deciphering or manipulation of any brain pattern or activity and various potential usages comprises a system or method for translating any brain activity or neurophysiological, neurological, functional or other pattern into identifiable semantic, syntactic, functional, action, sensing, perceiving, algorithmic or any other computational or other output (as described in FIG. 7); A system or method for translating any recognizable localized or dispersed brain activity or pattern into understandable output which also allows us to decipher any given brain activity state or function or transmission into a meaningful output; A system or method for utilizing such identifiable brain activity, function or state as means for monitoring, manipulating, enhancing or teaching that individual or brain new information, enhancing existing or other desirable states etc.; Such a system or method could be utilized for reacquiring lost cognitive, sensory, language, memory, physiological, motor, perceptual etc. information. It can also be used in order to interface with external computerized, robotic, artificial or human entities or devices. The system can also be used to heal, teach, improve undesirable or negative states of physiology, immunology, self-esteem, traumatic memories, loss of memory or physical, emotional or mental capacities or otherwise to enhance desirable skills, thought-patterns, or certain patterns or type of behavior.

A method or system or device for the manipulation of time-flow or materialization or de-materialization of objects or events or phenomena or phenomenon comprising a system and method for manipulating the flow of time, e.g., ‘forward’ or ‘backward’ (relative to its ‘normal’ known physical flow) through the application of an electromagnetic field that is either in the same direction as the electromagnetic field exerted on an object (on Earth or elsewhere) or is opposite to the object's electromagnetic field (on Earth or elsewhere); The relative pace of ‘forward’ or ‘reversed’ time flow can be controlled through the intensity and direction of the electromagnetic field or radiation relative to an object's own electromagnetic field such that if the direction and intensity of the electromagnetic field reverses the object's (or event's) original magnetic field it produces a reversal of the time flow for this object (or event), or alternatively if the exerted electromagnetic field or radiation increases the intensity or alters the direction of the object's existent electromagnetic field then it can advance the time-flow of this object or event (beyond its current space-time or quantum) value. A system and method for materialization or de-materialization of an object (or event) through an electromagnetic scanning of the object's precise electromagnetic values which can then be utilized through an application of an ‘opposite-valued’ electromagnetic field in order to de-materialize the desired object (or event) or to materialize this object or event at a different spatial-temporal localization (through the creation of that precise electromagnetic imprint at that particular localization). A system or method for altering an object or event's existent spatial-temporal value or localization through the abovementioned capacity to de-materialize or materialize an object or event, e.g., at any desired spatial-temporal point (in the universe). Such System or method also allows for the reversal or time-related aging, disease (and even death) processes or events of a given object/s or event/s and for the creation of new spatial-temporal objects or events based on the abovementioned principles of materialization or dematerialization. Alternatively (or additionally) A system or method which allows for the creation of novel objects or events through the application of novel electromagnetic field or direction constituting existing or new objects or events.

Notice is made to the possible connection or extension of the Computational Unified Field Theory (or the Duality Principle) described in this patent application as it may relate to the inventions mentioned in this document (or indeed any other existent or prospective inventions);

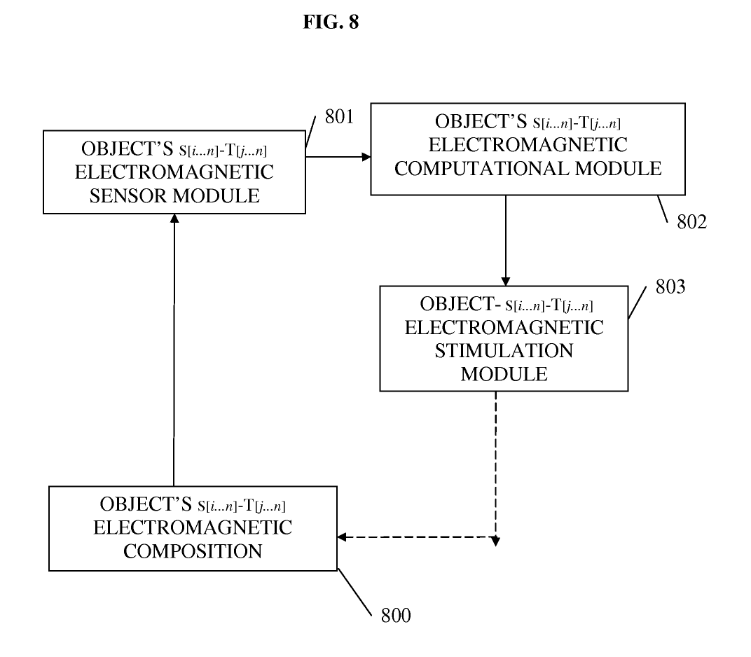

Reference is now made to FIG. 8 which describes an OBJECT'S S[I . . . N]−T[J . . . N] ELECTROMAGNETIC COMPOSITION 800 which is the total constituents electromagnetic values of an object, event or phenomena (at time: ti . . . n and spatial location or locations: si . . . n), which is detected, measured or identified by an OBJECT'S ELECTROMAGNETIC SENSOR MODULE 801, which is capable of identifying or measuring or determining the OBJECT'S ELECTROMAGNETIC COMPOSITION 800, which is then encoded, stored, transferred or transformed into any kind of computational representation (through any computational, communication, computer or robotic or human means or methods), which is then outputted to an OBJECT'S ELECTROMAGNETIC COMPUTATIONAL MODULE 802 that is capable of manipulating, computing or processing of any spatial-temporal or other dimension computation of that object, event, phenomena or spatial-temporal value, dynamics, change or transformation (existent or desired; norm, value or criteria based), which is then outputted to the OBJECT-S[i . . . n]−T[j . . . n] ELECTROMAGNETIC STIMULATION MODULE 803 that is capable of transforming, manipulating or acting upon the OBJECT'S S[I . . . N]−T[J . . . N] ELECTROMAGNETIC COMPOSITION 800 (at any temporal ti . . . n or spatial si . . . n value).

Since FIG. 8 delineates a potentially continuous process which can measure the OBJECT'S S[I . . . N]−T[J . . . N] ELECTROMAGNETIC COMPOSITION 800 through the OBJECT'S ELECTROMAGNETIC SENSOR MODULE 801 and carry out any possible comparison, computation or analysis of the object's past, present or prospective temporal ti . . . n or spatial si . . . n value/s carried out through the OBJECT'S ELECTROMAGNETIC COMPUTATIONAL MODULE 802, which can then be used to modulate, manipulate or otherwise determine the desired or past or future spatial-temporal electromagnetic values of the OBJECT'S S[I . . . N]−T[J . . . N] ELECTROMAGNETIC COMPOSITION 800 through the OBJECT-S[i . . . n]−T[j . . . n] ELECTROMAGNETIC STIMULATION MODULE 803 then this invention is capable of either reversing- or progressing- (e.g., beyond that object's existent spatial-temporal value/s at any S[i . . . n]−T[j . . . n] value or point) of the OBJECT'S S[i . . . n]−T[j . . . n] ELECTROMAGNETIC COMPOSITION 800; Alternatively, it may be possible to record, transfer, cause the disappearance of that object at any given spatial-temporal point and/or its transfer to any other spatial-temporal point (e.g., in the past, present or future etc.); Based on this operational principle it should also be possible to cause the appearance of the OBJECT'S S[I . . . N]−T[J . . . N] ELECTROMAGNETIC COMPOSITION 800 (e.g., object, phenomenon or phenomena, or spatial-temporal region) at multiple spatial-temporal points simultaneously or in any possible S[I . . . N]−T[J . . . N] combination or combinations; It should also be possible to cause the disappearance of the OBJECT'S S[I . . . N]−T[J . . . N] ELECTROMAGNETIC COMPOSITION 800 (at any possible spatial or temporal point or points) and then transfer the OBJECT'S S[I . . . N]−T[J . . . N] ELECTROMAGNETIC COMPOSITION 800 to any other (single or multiple) spatial-temporal point or points; In this manner it should also be possible to project or transfer or otherwise produce the OBJECT'S S[I . . . N]−T[J . . . N] ELECTROMAGNETIC COMPOSITION 800 at any single or multiple spatial-temporal points which would allow for the travelling, re-production, or transference of any OBJECT'S S[I . . . N]−T[J . . . N] ELECTROMAGNETIC COMPOSITION 800 (object/s or phenomena or phenomenon) at the speed of light or the speed of electromagnetic propagation.

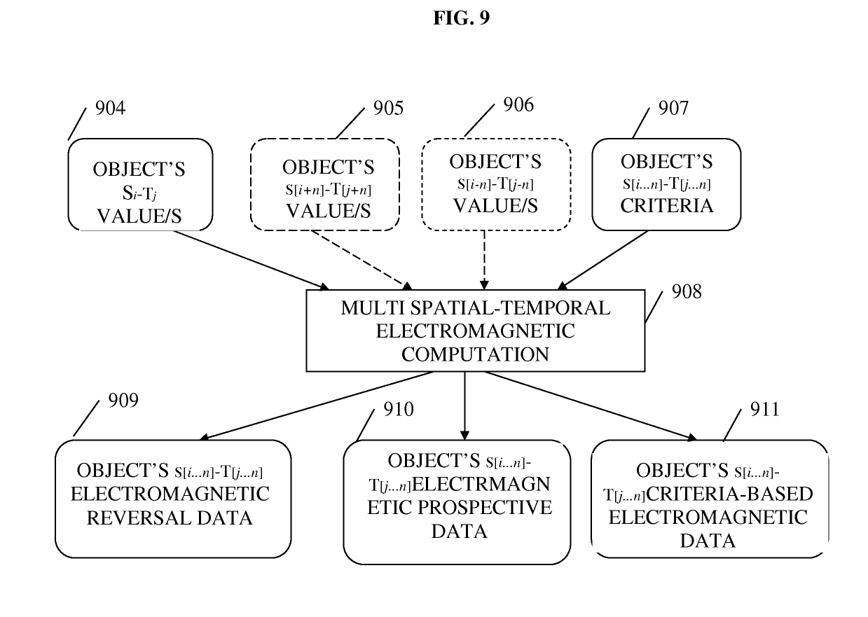

Indeed, FIG. 9 delineates the flow chart describing the operation or processing of the OBJECT'S S[i . . . n]−T[j . . . n] ELECTROMAGNETIC COMPUTATIONAL MODULE 802, whereby the OBJECT'S Si−Tj VALUE/S 904 which are the object's given spatial-temporal electromagnetic values at any given point in time and space, or the OBJECT'S S[i+n]−T[j+n] VALUE/S 905 which are the OBJECT'S S[I . . . N]−T[J . . . N] ELECTROMAGNETIC COMPOSITION 800 electromagnetic values at a prospective spatial-temporal point (e.g., relative to the object's given spatial-temporal electromagnetic value), or the OBJECT'S S[i−n]−T[j−n] VALUE/S 906 which relate to the object's electromagnetic values at a spatial-temporal prior point, or the OBJECT'S CRITERIA S[i . . . n]−T[j . . . n] 907 which relate to predetermined or criteria or desired electromagnetic values of an object or phenomenon or phenomena at any spatial-temporal point or points S[i . . . n]−T[j . . . n]; Each of these data sets, e.g., OBJECT'S CRITERIA S[i . . . n]−T[j . . . n], OBJECT'S S[i+n]−T[j+n] VALUE/S 905, OBJECT'S S[i−n]−T[j−n] VALUE/S 906, or OBJECT'S CRITERIA S[i . . . n]−T[j . . . n] 907 are inputted into the MULTI SPATIAL-TEMPORAL ELECTROMAGNETIC COMPUTATION 908 which is capable of comparing any given object's electromagnetic value/s at any given spatial-temporal point S[i . . . n]−T[j . . . n] with any other spatial-temporal point or points S[i . . . n]−T[j . . . n] or which may precede or follow these specific spatial-temporal (electromagnetic) values, or with a pre-determined or a criteria or a desired electromagnetic and/or any hypothetical spatial-temporal value. Based on this computation by the MULTI SPATIAL-TEMPORAL ELECTROMAGNETIC COMPUTATION 908 which takes into consideration or computes or determines the electromagnetic value/s of an object at a given spatial-temporal point/s S[i . . . n]−T[j . . . n] or compares between an object's given spatial-temporal point or points S[i]−T[j] electromagnetic value and other spatial-temporal point's electromagnetic value either before S[i . . . n]−T[j . . . n], or after S[i+n]−T[j+n] the given spatial-temporal electromagnetic value, or compares between the object/s electromagnetic value at any given spatial-temporal point/s and a given or pre-determined or criteria or desired electromagnetic value/s and/or spatial-temporal value/s S[i . . . n]−T[j . . . n] the MULTI SPATIAL-TEMPORAL ELECTROMAGNETIC COMPUTATION 908 outputs an OBJECT'S S[i . . . n]−T[j . . . n] ELECTROMAGNETIC REVERSAL DATA 909, which refers to either the electromagnetic stimulation data necessary to affect a reversal in an object's spatial-temporal and/or electromagnetic value/s or to the object's previous spatial-temporal S[i−n]−T[j−n] and/or electromagnetic value/s. Likewise the SPATIAL-TEMPORAL ELECTROMAGNETIC COMPUTATION 908 outputs an OBJECT'S S[i+n]−T[j+n] PROSPECTIVE ELECTROMAGNETIC DATA 909 which refers to either the electromagnetic stimulation data necessary to affect changes in the ‘future’ of an object's spatial-temporal and/or electromagnetic value/s or which informs or determines or records the object's prospective spatial-temporal S[i−n]−T[j−n] and/or electromagnetic value/s. Likewise the SPATIAL-TEMPORAL ELECTROMAGNETIC COMPUTATION 908 outputs an OBJECT'S S[i+n]−T[j+n] CRITERIA-BASED ELECTROMAGNETIC DATA 911 which refers to either the electromagnetic stimulation data necessary to affect changes in the spatial-temporal and/or electromagnetic value/s of an object in order to attain a predetermined or criteria-based or any desired spatial-temporal and/or electromagnetic value/s or which informs or determines or records the object's prospective spatial-temporal S[i−n]−T[j−n] and/or electromagnetic value/s based on a certain criteria etc.

One possible embodiment of the invention involves the MULTI SPATIAL-TEMPORAL ELECTROMAGNETIC COMPUTATION 908 determination of the OBJECT'S S[i . . . n]−T[j . . . n] ELECTROMAGNETIC REVERSAL DATA 909 through a computation of an electromagnetic stimulation of the object that is opposite in direction and/or in intensity, frequency or any other physical attribute associated with the electromagnetic field, strength, direction, intensity, duration etc. of the object (which can be measured at a single spatial-temporal point or across multiple spatial-temporal points S[i . . . n]−T[j . . . n] and consequently applied also at a single spatial-temporal point or points or across a series of (static or dynamic) electromagnetic point/s; Based on the Computational Unified Field Theory in the Appendix it is possible that any object's appearance across a series of universal simultaneous computational frames ‘USCF’ is associated with the speed of light and/or light as an electromagnetic field of wave that underlies or is associated with the succession—forward or backwards of these USCF's, it is plausible to assume that an application of a magnetic field, light or any other electromagnetic radiation, effects etc. on an object at a particular direction, intensity, duration or any other physical property of the electro-magnetic field or of the object (e.g., whether in opposite direction and/or intensity to the electromagnetic field or value/s of the object or in any other conceivable formulation) may lead to a reversal of the succession of frames, e.g., either for that particular object or for a certain segment (or even the totality) of the USCF's associated with that object. In this manner through the application of an electromagnetic field, force, wave or other associated electromagnetic or other associated physical property of the object—applied to the object either at a single or multiple spatial-temporal points it may be possible to reverse the flow of spatial-temporal events of values or electromagnetic properties of that object.

Likewise, One possible embodiment of the invention involves the MULTI SPATIAL-TEMPORAL ELECTROMAGNETIC COMPUTATION 908 determination of the OBJECT'S S[i+n]−T[j+n] PROSPECTIVE ELECTROMAGNETIC DATA 909 may be achieved through a computation of an electromagnetic stimulation of the object that is either in the same direction of the object at a particular spatial-temporal point or points (or in a different direction than the object's electromagnetic field of value or direction at a particular spatial-temporal point or points) with the same or different electromagnetic intensity, duration, or any other physical property associated with the electromagnetic field, strength, direction, intensity, duration etc. of the object (which can be measured at a single spatial-temporal point or across multiple spatial-temporal points S[i . . . n]−T[j . . . n] and consequently applied also at a single spatial-temporal point or points or across a series of (static or dynamic) electromagnetic point/s; Based on the Computational Unified Field Theory in the Appendix it is possible that any object's appearance across a series of universal simultaneous computational frames ‘USCF’ is associated with the speed of light and/or light as an electromagnetic field of wave that underlies or is associated with the succession—forward of these USCF's, it is plausible to assume that an application of a magnetic field, light or any other electromagnetic radiation, effects etc. on an object at a particular direction, intensity, duration or any other physical property of the electro-magnetic field or of the object (e.g., whether in opposite direction and/or intensity to the electromagnetic field or value/s of the object or in any other conceivable formulation) may lead to the appearance of the object's prospective spatial-temporal point/s or values, e.g., either for that particular object or for a certain segment (or even the totality) of the USCF's associated with that object. In this manner through the application of an electromagnetic field, force, wave or other associated electromagnetic or other associated physical property of the object—applied to the object either at a single or multiple spatial-temporal points it may be possible to reverse the flow of spatial-temporal events of values or electromagnetic properties of that object.

Likewise, One possible embodiment of the invention involves the MULTI SPATIAL-TEMPORAL ELECTROMAGNETIC COMPUTATION 908 determination of the OBJECT'S S[i+n]−T[j+n] CRITERIA-BASED ELECTROMAGNETIC DATA 911 may be achieved through a computation of an electromagnetic stimulation of the object based on predetermined, or criteria-based or desired values in comparison with the object's single or multiple spatial-temporal point or points (or in a different direction than the object's electromagnetic field of value or direction at a particular spatial-temporal point or points) value/s—relative to the criteria or desired spatial-temporal and/or electromagnetic value/s which are to be achieved; This (single spatial-temporal or multiple spatial-temporal points) computation then allows to derive the precise spatial-temporal and/or electromagnetic stimulation parameters necessary to manipulate the spatial-temporal and/or electromagnetic values or properties of the object across a single or multiple spatial-temporal points; Hence, it should be possible for the MULTI SPATIAL-TEMPORAL ELECTROMAGNETIC COMPUTATION 908 to determine the OBJECT'S S[i+n]−T[j+n] CRITERIA-BASED ELECTROMAGNETIC DATA stimulation parameters which may allow to produce or reproduce any given object (or even any non-existent electromagnetic object, property, phenomena or phenomenon) at any single or multiple spatial-temporal points, thus in effect creating “electromagnetic” (or real) replicates or “clones” of the same object at different (or the same) spatial-temporal values.

Moreover, based on the MULTI SPATIAL-TEMPORAL ELECTROMAGNETIC COMPUTATION 908 capacity to determine the OBJECT'S S[i+n]−T[j+n] CRITERIA-BASED ELECTROMAGNETIC DATA which allows for the stimulation parameters or the information regarding the object's appearance or dis-appearance at any particular spatial-temporal point or points indeed affords the possibility of altering the electromagnetic or indeed physical make-up, spatial-temporal, or sequence of events or phenomena associated with an object/s; Alternatively this capacity allows to eliminate or cause the disappearance of an object at a particular electromagnetic and/or spatial-temporal value or location and/or reproduce the object's ‘past’ ‘present’ or ‘future’ e.g., at any hypothetical single or multiple spatial-temporal and/or electromagnetic value or location etc.

Likewise, the MULTI SPATIAL-TEMPORAL ELECTROMAGNETIC COMPUTATION 908 capacity to determine the OBJECT'S S[i+n]−T[j+n] CRITERIA-BASED ELECTROMAGNETIC DATA allows to mobilize or replace, transport, eliminate or reproduce the desired object/s or phenomena or phenomenon at any single or multiple spatial-temporal point/s at any location throughout the physical universe (in the ‘past’, ‘present’ or ‘future’—as defined above) (at a velocity or speed that may be up to the speed of light); This would imply that an object such as a plane, human body or any other object or objects could be transported at up to the speed of light across any distance (provided off course that we are capable of reproducing the same or other electromagnetic stimulation or field as of the object or phenomena that we wish to create, re-produce or modify at any spatial-temporal point/s; It may thus be possible to acquire a person's or object/s electromagnetic value/s at a given spatial-temporal point/s then stimulate that object or human being with (opposite direction or the same or other direction f electromagnetic stimulation) in order to either eliminate the appearance of that object or human being from a particular spatial-temporal point/s and/or produce or reproduce that object or human at any other or same spatial-temporal location and/or at a different or same electromagnetic value. Such formulation would allow to transport humans or any other object or phenomena to any spatial-temporal location or to alter the electromagnetic field or value of that object/s at any spatial-temporal location or value.

Finally, by the transference of this output data from the MULTI SPATIAL-TEMPORAL ELECTROMAGNETIC COMPUTATION 908 to the OBJECT-S[i . . . n]−T[j . . . n] ELECTROMAGNETIC STIMULATION MODULE it should be possible to accomplish all of the abovementioned effects or transformation or transport or reversal or prospective spatial-temporal and electromagnetic value/s through its application to the OBJECT'S S[i . . . n]−T[j . . . n] ELECTROMAGNETIC COMPOSITION. Hence, it should be possible to reverse the flow of spatial-temporal events for any object, or to predict or even to produce the prospective spatial-temporal and/or electromagnetic values of any given or hypothetical object or event or phenomena, to reverse any disease process, or potentially even reverse critical events such as death, birth, injury, deformation, loss of any cognitive or physiological or emotional or behavioral or any other physical or biological or other phenomena through the application of the abovementioned certain electromagnetic field at any given single or multiple spatial-temporal point/s.

In a preferred embodiment of the invention the object whose OBJECT'S S[i . . . n]−T[j . . . n] ELECTROMAGNETIC COMPOSITION is being manipulated such as the human body or indeed select parts or organs, specific single or multiple regions of the body, brain, tissue or tissues, or more generally any object or phenomena or phenomenon may be placed within a device or an apparatus which is capable of measuring the electromagnetic field (e.g., intensity, direction, fluctuation, or any other physical property associated with the magnetic field and/or its spatial-temporal dynamics or characteristics and/or any other physical property including the energy, mass, temporal characteristics or spatial values or dimensions of the object which may be associated with a change in the electromagnetic field of values of an object) or value or values of that object at single, multiple or the entirety of its composition—before, during and after the application of any electromagnetic stimulation (which may alter the subsequent electromagnetic and/or spatial-temporal, energetic, spatial, mass etc. of the organ, body part or human body or any other object to which the electromagnetic stimulation is applied. Such preferred embodiment is also capable of computing the appropriate properties of the electromagnetic stimulation (as described above including but not limited to intensity, duration, localization, or dynamic fluctuation in time or space or any other physical property) which may be necessary to reverse the flow of time or spatial-temporal events or occurrences or processes or progress the spatial-temporal events, occurrences processes etc., or that are necessary in order to eliminate the appearance of an object or its electromagnetic value or values and produce or replicate or reproduce (simultaneously or at different spatial-temporal point or points) the object or even parts or elements of the object, or may even create new objects based on a specification of their electromagnetic and/or spatial-temporal values or characteristics, and/or electromagnetic and/or spatial-temporal or any other physical property (energy, mass, space, time, wave or particle etc/) of the stimulated object or parts of it; Hence, such preferred embodiment may also “clone” or replicate an object, organ, gene, brain activity pattern that may be characteristic of a specific thought, processing capacity or skill etc. (which may be superimposed on implanted electromagnetically and/or spatial-temporally into the same or other spatial-temporal locations or objects etc. (which may also regenerate tissues, diseases etc.) or conversely may be used in order to weaken or eliminate any antigen, disease agent cancerous cells or any aberrant phenomena, spatial-temporal occurrence etc.; viruses may be weakened selectively, cancerous cells targeted to be eliminated or weakened etc. Such preferred embodiment may therefore consist of the production of an electromagnetic field around an object, human body, organ, cell or even microelements such as genes molecules, nerves sub-cellular elements—based on an analysis of the desired spatial-temporal or electromagnetic reversal, progressing, elimination or production, reproduction at single or multiple spatial-temporal points throughout the universe or production of novel electromagnetic field/s, object/s phenomena, or which may be used for the reversal or reoccurrence of disease (birth or death) etc.

One such possible embodiment may be the placing of an object within a tube or an engulfing (partial or complete) magnet which is capable of either producing weak or strong electromagnetic fields which can be directed towards any part/s of the object and induce any sort of electromagnetic stimulation properties including (but not limited to) specification of intensity, direction, duration, spatial-temporal fluctuation etc. which are based on an additional measurement of the object's static or dynamic (single or multiple spatial-temporal) of electromagnetic, and/or spatial-temporal and or any other physical measurement and/or determination or computation of the particular spatial-temporal or electromagnetic stimulation necessary in order to alter the spatial-temporal sequence of events or electromagnetic properties (relating to space, time, energy or mass etc. or to biological processes of health healing or disease, birth death, or any undesired accidents, injuries, or desired events etc.), or the production or elimination of an object, event, or parts or processes or elements (biological, physical microscopic, genetic, viral, immunological, neurological atmospheric etc.), or the reproduction of an object at single or multiple simultaneous or non-simultaneous spatial-temporal point/s. [In case the electromagnetic stimulation of an object/s may cause a spatial-temporal transportation or change in that object/s or event/s (or surrounding environment or associated spatial-temporal object/s or event's or phenomena) it may be necessary to design this invention in such a manner which allows for a continuous monitoring, measurement and/or electromagnetic stimulation so that the objective of a continuous altering of the electromagnetic and/or spatial-temporal characteristics or location/s of the object/s would not be interrupted by such changes. Finally, another embodiment of the present invention relates to the possibility of transporting an object, person/s plane or Earth transportation or space vehicle by means of this analysis of the constituents of the object/person/s (or organ/s) etc. at the speed of light or below to different locations through this system which allows to scan, measure eliminate reproduce and hence also transport object/s to distant (or near) regions of space at which the same (or different) electromagnetic imprints can be produced!

In another possible embodiment of the invention, the OBJECT-S[i . . . n]−T[j . . . n] ELECTROMAGNETIC STIMULATION MODULE may produce an electromagnetic field that reinforces, strengths or otherwise enhances the already existent OBJECT'S S[i . . . n]−T[j . . . n] ELECTROMAGNETIC COMPOSITION based on the OBJECT'S S[i . . . n]−T[j . . . n] ELECTROMAGNETIC SENSOR MODULE input and OBJECT'S S[i . . . n]−T[j . . . n] ELECTROMAGNETIC COMPUTATIONAL MODULE. Such embodiment would allow for aversion of disease, e.g., of any type of associated with a particular organ or body part which may be stimulated separately or in conjunction with any other body part or whole body stimulation, or for enhancement of health, well-being, physical, emotional, behavioral capacities. The abovementioned embodiment of the invention may involve electromagnetic stimulation which is applies in the same or other direction, intensity or any other physical property involving the already existent magnetic field of the object.

A system or method for controlling global warming atmospheric and climate change and for transformation of electromagnetic radiation into usable energy comprising a system and method for cooling the Earth's atmosphere by means of a sheath covering of parts of or the entirety of the Earth's atmosphere which allows for the determination of partial or full obstruction of electromagnetic or heat related radiation passage into the Earth's atmosphere at specific loci; Alternatively, a system and method for partial or complete absorption of electromagnetic, heat, light or other sources of energy received by the Earth's atmosphere through intra-atmospheric, extra-atmospheric or Earth-surface absorption sheaths or mediators. Alternatively (or additionally) such a system can consist of (single or multiple) sources of focal energy stimulation of specific loci in the atmosphere or on the surface of the Earth which may be produced by electromagnetic, light, heat or other forms of energy stimulators that may be placed (or orbit) at any point within or outside of the Earth's atmosphere, or alternatively on the surface of the Earth; Either (or both) of the abovementioned systems—e.g., for partial/full obstruction of the electromagnetic (or light) extra-atmospheric radiation or for producing focal electromagnetic, light, heat (or other) energy stimulation of specific regions in the Earth's atmosphere or specific regions on the Earth's surface can be controlled or modulated through a centralized computing module via a computerized system which can determine which region or regions in the Earth's atmosphere or surface should be either diminished in terms of its received electromagnetic, light, heat or other radiation or alternatively should receive an enhanced electromagnetic, light, heat or other (focal—single region or multiple regions) radiation. Such a system which can either modulate the degree of extra-atmospheric radiation penetration, intra-atmospheric absorption gradient, or production of atmospheric or Earth-surface focal radiation (in any possible combination) allows for cooling of the Earth's atmosphere or surface and also allows for an effective intervention in the Earth's atmospheric patterns of weather changes, precipitation or extreme weather conditions (e.g., such as tornadoes, hurricanes etc.)

Finally, the invention also claims for a system and method for transformation of intra- or extra-atmospheric electromagnetic, heat, light or other radiation or sources of energy into usable electrical or other forms of usable energy.

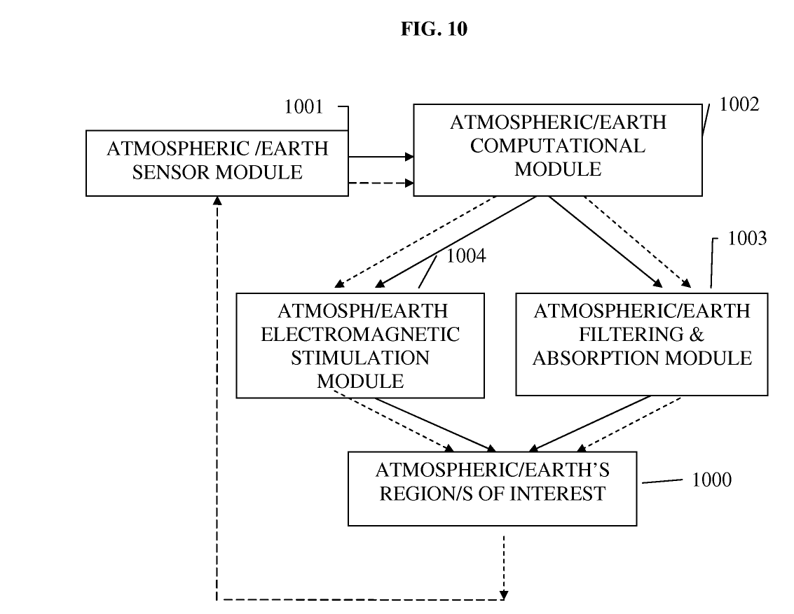

Reference is now made to FIG. 10 which describes an ATMOSPHERIC/EARTH'S REGION/S OF INTEREST 1000 which refers to any single or multiple region or regions, point or points within the Earth's atmosphere or on the Earth' surface or even any point/s or regions within the Earth's sub-surface, planet etc. which is of interest within the scope of this invention and/or is related to the measurement or assessment of any physical property associated with the Earth's surface, atmosphere or sub-surface region/s such as heat, electromagnetic field, seismic activity or property, sound, volcanic activity, whether patterns, humidity, wind, air-pressure, air or water or heat or other currents or global patterns of heat, weather, liquid/s, precipitation, earth-quake, temperature, sea-water level, atmospheric- whether, air- or water- or ocean/current turbulence etc. or any other extra-planetary cosmic rays, radiation, etc., intra-galactic ‘wind’, meteors etc., temperature, or any other localized or diffused property of the Earth's atmosphere, surface or interior (sub-surface) [Note that precisely the same technology can be applied to any other planet in the universe including applications which may derive or produce atmospheric, water, humidity or the formation of conditions necessary to cool, heat, produce water or any other chemical or thermal conditions on another planet—e.g., which may be conducive for the development of life on that planet or for the habituation of that planet or surrounding that planet with humans or other life forms];

These ATMOSPHERIC/EARTH'S REGION/S OF INTEREST 1000 are being measured or sensed by or through an ATMOSPHERIC/EARTH SENSOR MODULE 1001 which is a measurement device or devices or apparatus or apparatuses capable of measuring the abovementioned ATMOSPHERIC/EARTH'S REGION/S OF INTEREST 1000, e.g., any single or multiple region or regions, point or points within the Earth's atmosphere or on the Earth' surface or even any point/s or regions within the Earth's sub-surface, planet etc. which is of interest within the scope of this invention and/or is related to the measurement or assessment of any physical property associated with the Earth's surface, atmosphere or sub-surface region/s such as heat, electromagnetic field, seismic activity or property, sound, volcanic activity, whether patterns, humidity, wind, air-pressure, air or water or heat or other currents or global patterns of heat, weather, liquid/s, precipitation, earth-quake, temperature, sea-water level, atmospheric- whether, air- or water- or ocean/current turbulence etc. or any other localized or diffused property of the Earth's atmosphere, surface or interior (sub-surface). Hence, the ATMOSPHERIC/EARTH SENSOR MODULE 1001 is capable of measuring single or multiple of Earth's (or indeed any other planet's) Atmospheric, surface or sub-surface physical properties (or in the case of other planets what may be considered their extra-planet physical properties such as extra-planetary cosmic rays, radiation, etc., intra-galactic ‘wind’, meteors etc., temperature, or any other localized or diffused property of the planet's atmosphere, surface or interior (sub-surface), life-signs or potential etc.) and/or at different spatial-temporal points and/or at single or multiple spatial-temporal points or values; The ATMOSPHERIC/EARTH SENSOR MODULE 1001 is also capable of measuring, recording, computing, encoding or storing or manipulating or transferring or broadcasting or performing any digital, analogue, computerized or any other conceivable storage, manipulation, transfer of information or communication function, procedure or process or processing with this information; Indeed, the ATMOSPHERIC/EARTH SENSOR MODULE 1001 transfers its abovementioned information, operation, encoding, functions etc. to the ATMOSPHERIC/EARTH COMPUTATIONAL MODULE 1002 which is capable of computing, comparing, analyzing, storing, sorting, computing any single or multiple spatial-temporal patterns, values, occurrences, criteria-based analysis or analyses or computation/s associated with the abovementioned physical properties measured by the ATMOSPHERIC/EARTH SENSOR MODULE 1001; One preferred embodiment of the ATMOSPHERIC/EARTH COMPUTATIONAL MODULE 1002 is capable of computing any global or local spatial-temporal patterns, distribution, atmospheric, heat, temperature, humidity, seismic, volcanic, inter- or intra-tectonic plates or activity, precipitation, air pressure systems, winds, liquid or liquidity patterns or whether patterns, e.g. either globally or regionally, and determine the connections, dynamics, localization of low vs. high barometric pressure, dry vs., humid or precipitation levels, global warming levels or spots, holes or degradation of ozone layers, cosmic rays or other forms of radiation or any other chemical, atmospheric, electromagnetic, heat or any other patterns associated with the Earth's (or any other planet as discussed above) atmosphere, surface or sub-surface interior or seismic or other patterns. Thus, for instance, the ATMOSPHERIC/EARTH COMPUTATIONAL MODULE 1002 is capable of determining for each region or regions or loci or locus of the Earth's (or other planet's) atmospheric, surface or sub-surface point/s the precise levels of electromagnetic, heat, air-pressure, humidity, radiation or any other physical property values or characteristics, and/or determine the single or multiple relationship/s between each of these numerous physical properties at single or multiple atmospheric-surface- or sub-surface (as described above) single or multiple spatial-temporal relationships with any other spatial-temporal point across the Earth's (or other planet's) atmospheric, surface or sub-surface etc. point/s regions etc. patterns or dynamics or interrelationships. Likewise, the ATMOSPHERIC/EARTH COMPUTATIONAL MODULE 1002 is also capable of contrasting, comparing or otherwise analyzing (in any possible manner) any of these single or multiple spatial-temporal loci or locus to any given atmospheric, seismic, volcanic, tectonic plates, whether, barometric, precipitation conditions or values, storms hurricanes, tornadoes, earth-quakes, global warming measure etc., and/or also to compute the necessary electromagnetic, heat or other forms of stimulation or stimulating or intervention in the Earth's single or multiple point/s or regions of the atmosphere's or surface or sub-surface or extra-atmospheric regions in order to attain a certain value, criteria, condition or outcome etc. such as formation of precipitation, annihilation of a hurricane, tornado, tropical or other storm, seismic activity, earth-quake, rise or drop in sea-level, humidity, barometric pressure or any other physical condition or property of the Earth or its (internal or external) environment. This would also include the alteration of physical conditions associated with any bio-system such as forestation or de-forestation, liquid formation, gaseous conditions, ozone layer associated factors or amending any holes in the Earth's atmospheric shield, Ozone or other elements protective properties or capacities etc., greenhouse effects etc.

Thus, for instance it should be possible with such or similar embodiments of the invention to produce, alter, protect, reproduce, shift or ameliorate any drought or excessive rain, sea level, excited or instable atmospheric conditions in a particular or generalized regions or regions or even the entirety of the Earth's atmosphere or sub/surface regions—through a computation of the precise level or levels or condition/s related to any of the possible physical properties of the Earth or its atmosphere etc. in a single or multiple regions of the Earth's atmosphere or surface or sub-surface regions that are necessary in order to cause precipitation in any specific region, or to dissipate a hurricane or immanent volcanic activity, unstable Earth weather or oceanic or water sea-level condition etc. Hence, for instance it should be possible to cause the formation of water-bearing clouds or atmospheric conditions at a particular atmospheric region in the atmosphere adjacent or above a water source—then compute the conditions necessary to mobilize or displace that humidity or liquid or water evaporation entity or process to travel or be displaced to a particular desired regions or desert of agricultural or other region on the Earth's surface and/or compute the necessary conditions in order to cause or bring about precipitation in that desired location/or locations etc Likewise, it should be possible with such or similar embodiments to compute the necessary interventions for dissipating or disrupting or otherwise canceling or diminishing any seismic, volcanic or undesired weather system such as a tornado, hurricane etc. through the computation of the necessary change in single or multiple atmospheric, Earth's surface or sub-surface point/s or region/s (which could alter in one embodiment the barometric, temperature, humidity etc. or any other physical localized or un-localized property associated with such weather seismic or other phenomena. Likewise it should be possible to calculate, compute or determine the precise ill-effects or otherwise undesired causes, factors contributing to- or effects of-‘global warming’ or any associated factors such as the localized or generalized atmospheric, Earth's surface or sub-surface temperature, physical properties, radiation etc. and hence also be able to compute, calculate or otherwise determine the precise single or multiple regions of the atmosphere or surface that need to be either stimulated electromagnetically, heat or any other form of stimulation or alternatively need to be shielded, degraded or modulated—downwards in terms of the intensity of absorbed (or emitted) heat, electromagnetic, sun, light or any other form of electromagnetic or other form of radiation or heat, light, ultraviolet or other electromagnetic radiation etc.

Hence, based on the ATMOSPHERIC/EARTH COMPUTATIONAL MODULE's 1002 computation or processing or any sort of output determines or controls or informs or instructs or operates the functioning of the

ATMOSPH/EARTH ELECTROMAGNETIC STIMULATION MODULE 1004 which is capable of stimulating electromagnetically or otherwise in any other sort or type of heat, light, sound, seismic, volcanic, or any other form of stimulation of any single or multiple region/s or the Earth's surface, sub-surface, atmospheric etc. region/s or point/s. Alternatively or in combination with the abovementioned embodiment of the invention based on the ATMOSPHERIC/EARTH COMPUTATIONAL MODULE's computation or processing or any sort of output determines or controls or informs or instructs or operates the functioning of the ATMOSPHERIC/EARTH FILTERING & ABSORPTION MODULE 1003 which is capable of filtering, absorbing, degrading, or otherwise modulating the degree or existence or levels of electromagnetic or any other form/s of radiation, heat, seismic activity or any other physical activity etc. at any single or multiple Earth's surface or sub-surface or atmospheric point/s and/or in order to obtain any desired or criteria condition, modify existing conditions or producing or re-producing any desired physical conditions etc.; The ATMOSPHERIC/EARTH COMPUTATIONAL MODULE's computation or processing or any sort of output which determines or controls or informs or instructs or operates the functioning of the ATMOSPH/EARTH ELECTROMAGNETIC STIMULATION MODULE and/or ATMOSPHERIC/EARTH COMPUTATIONAL MODULE's allows for the modification of the ATMOSPHERIC/EARTH'S REGION/S OF INTEREST 1000 in any way possible including but not limited to any of the abovementioned embodiments, descriptions or delineations and/or allows for the control of global warming, weather, precipitation, seismic production of electromagnetic, electrical, heat or other forms of energy for usage at any point/s on Earth or in its atmosphere.

A system or method or medical device and/or smart functional electrode/s stimulation of CNS or bodily diseases and/or the Specificity Principle, personalized brain medicine, cortical and/or subcortical controlling brain diseases or enhancement/s treatments, critical pathways or junctions or regions for control or optimization or enhancement of human functioning, health, or therapeutic for any diseased state or brain condition comprising:

a system, method, medical device or apparatus or any other mechanism or mechanisms capable of deciphering or decoding or analyzing or determining or computing the significance, meaning, correlate (e.g., including but not limited to: behavioral, cognitive, affective, motor, physiological, input or response or action or potential or potential for any brain related or cognitive or physiological or any other bodily function or action or mechanism or function etc.) of neural- or single- or multiple-neuron/s or neural network- or neuronal- or electrophysiological- or electromagnetic- or electrical- or neurophysiologic- or brain-activity, pattern, function, operation, or characteristic/s; this system, method, functional capacity, computational capacity, system, loop smart electrode capacity or any other mechanism or apparatus (including the possibility of such brain or neuronal or neuronal or neuron or neurons or neural network/s deciphering system or method or medical device being capable of being a part of- or interfacing with- any brain stimulation or cognitive or affective or neuronal stimulation or manipulation or input- or output to brain or neuronal activity etc. including but not limited to the possibility of forming ‘sensory-response controlling loops’ of any identified diseased brain function, brain disease or diseased brain region/s or impaired brain or neuron function/s or aspect/s etc. (or even for enhancing normal brain, cognitive, affective, personality, neural or other brain, cognitive or neural etc. functions) that are capable of identifying or deciphering or localizing any diseased (or to be enhanced normal) brain or neuronal or cognitive or affective or motor or neurophysiologic function/s or region/s and then of manipulating or altering or healing or therapeutically affecting positively any such impaired or diseased neural, neuronal, neuron's or neuron's or neural-network/s abnormal or diseased or impaired of dysfunctional or sub-normal or to enhance beyond normal functioning (including the possibility of forming such ‘sensory-response controlling loops’ that can continuously or intermittently or adjustably or in a fixed manner work or stimulate or modulate the specific relevant neuron's or neurons' or neural network/s' or brain diseased or impaired function/s (including until a certain target brain functioning or neural functioning or any brain related disease/s or diseased or impaired neural function improvement or until a certain enhancement of any brain function/s or cognitive or affective or neuronal or other function/s or feature of normal brain functioning is enhanced to a certain degree or up to a certain threshold etc.) is achieved or until a certain improvement or threshold is reached). The described brain or neuronal or neural or cognitive or affective or motor or neurophysiologic or any other brain or neural correlate or function or output or input deciphering system or method or medical device or computational loop is based on the capacity to localize precisely specific single or multiple brain region/s or neuron/s or neural network/s or any specific locus or loci in the brain or in any neuron that is correlated or associated with a particular neural activity or neuronal activity or activities or function or functions or electrophysiological pattern or activity or activities or function/s or electrical or electromagnetic activity or activities and/or with any particular cognitive-affective-motor-neurophysiologic-electrophysiological process or processes or function/s or operation/s or lack of function/s or operation/s or processes, which allows to decode or decipher or interpret or determine or compute the precise meaning or correlate or significance or function or process or input or output or delineation or discovery or unraveling of any brain- or neural- or neuronal or brain- or any single or multiple neuron/s or neural-network/s-function, activity, operation/s, input, output, process/es, encoding, storage, processing, computation, cognition, affect, motor or neurophysiologic or any bodily physiological or biological function/s, activity/ies or occurrences or phenomena. The system described may also employ or consist of the manipulation of stimulus or stimuli or response/s directed to specific single or multiple brain region/s or neuron/s or locus or loci etc. or brain or neural function/s through the inhibition or facilitation of any specific single or multiple brain or neural function/s etc. which interfaces with the sensory measurement of time- and/or spatial- and/or computational-index or indices or function/s or operation/s or pattern/s etc. or activation, inhibition, facilitation or halting of these single or multiple brain or neural or cognitive, affective, motor etc. function/s or operation/s or process/es in order to accurately decipher or determine the precise significance or meaning or function of any single or multiple joint or dis-joint or associated or correlated or uncorrelated spatial- and/or temporal and/or computational- or affective- or cognitive- or neurophysiologic-event or activity/ies or activation or function/s.

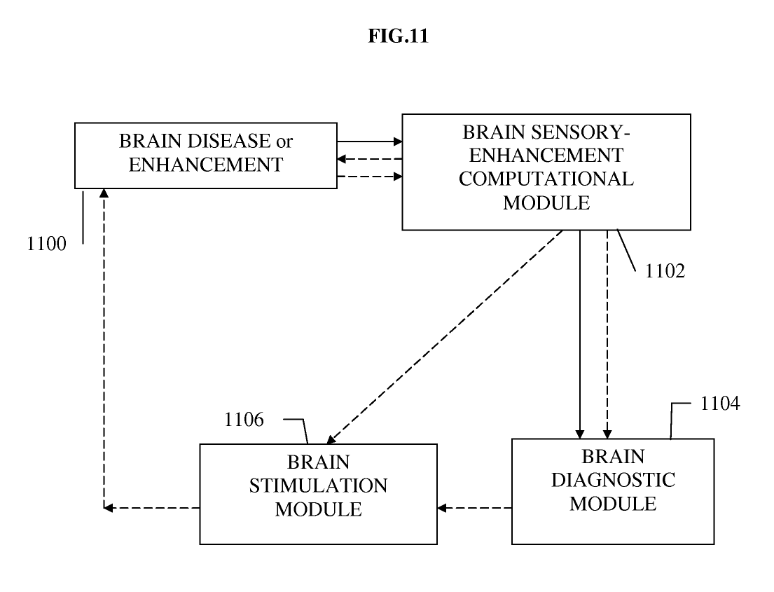

Reference is now made to FIG. 11 which describes a BRAIN DISEASE OR ENHANCEMENT 100 which refers to any single or multiple brain region/s or point/s or a single or multiple neuron/s or neuronal connection/s or pathway/s or any single or multiple brain or (central nervous system or peripheral nervous system) neural or neuronal or neuron/s that exhibit any disease or abnormality or impairment or deficiency or dysfunction of sub-normal functioning or structure or function or connectivity or electrophysiology or neurophysiologic features or cognitive or affective or motor or behavioral or physiological or intellectual or memory or processing or any other brain related or behavioral or neuronal deficiency or abnormality or disease or illness, or alternatively any such brain or neural or neuronal or any single or multiple brain region/s or point/s or a single or multiple neuron/s or neuronal connection/s or pathway/s or any single or multiple brain or (central nervous system or peripheral nervous system) neural or neuronal or neuron/s that are in need or are targeted for enhancement of function/s or cognitive or affective or intellectual or motor or physiological etc. or for enhancement of intelligence, affective health or other affective enhancement, personality, or any personality or cognitive or affective or behavioral enhancement (including but not limited to self-esteem, self-confidence, empowerment, mental health, etc., intellectual capacities etc.). The BRAIN DISEASE OR ENHANCEMENT 1100's single or multiple brain or neural or cognitive or affective or motor or behavioral or intellectual or any other brain or neural single or multiple region/s or cell/s or neuron/s or neural network/s diseased state or disease/s or abnormal feature/s or dysfunction/s or impairment/s or alternatively—enhancement or improvement (e.g., in individuals who exhibit any of these single or multiple brain, neural, cognitive, affective, behavioral, brain etc. features—which may be at normal- or sub-normal- or above-normal-functioning, level, threshold/s etc.) is being sensed or measured or identified or analyzed or data collected or various single or multiple continuous or intermittent or haphazard or methodical or other collection of single or multiple spatial- and/or temporal- or computational measures related or obtained or gathered from these single or multiple properties or features or aspects of brain, neural, cognitive, affective, motor, physiological, neurophysiologic etc. pattern/s by the BRAIN SENSORY-ENHANCEMENT COMPUTATIONAL MODULE 1102 which is also responsible for analyzing, computing or determining or figuring out or identifying any single or multiple brain or neural or neuronal or neuron/s or neural network/s or computational or cognitive or affective or behavioral or motor or physiological or neurophysiologic or intellectual or electrophysiological or any of these feature/s or measurement/s correlates or associations or causing or symptom/s or indication/s of any brain related or cognitive or affective or behavioral etc. disease/s illness/es or impairment/s or abnormality or dysfunction/s or alternatively determine the precise function/s or region/s or neuron/s or behavior/s, cognitive, affective, motor or physiological or intellectual, personality trait/s capacity/ies etc. is to be enhanced or trained etc. The BRAIN SENSORY-ENHANCEMENT COMPUTATIONAL MODULE 1102 initiates a series of computational-analysis/es-sensory- and other processes and function/s which allows it to decipher the precise meaning, significance, correlate/s, identification or analysis and/or determination of any single or multiple brain or neural or neuronal or neuron's or neurons' or cognitive or affective or neurophysiologic or motor or other brain-related which may be abnormal or diseased or which are normal or above-normal; Such brain or neural deciphering or quantification or computation or determination of the BRAIN SENSORY-ENHANCEMENT COMPUTATIONAL MODULE 1102 is hence capable of deciphering any single or multiple spatial- or and/or temporal and/or computational- and/or affective and/or cognitive and/or neurophysiologic and/or behavioral and/or any other brain or neural or (central or peripheral) neural activity including its meaning or function or operation or input or output or (behavioral, cognitive, motor or other neural or brain or cognitive, affective action, potential or operation or other) correlate/s or association or causal or result etc., which may also be utilized for the purpose of identifying any diseased brain or neuron/s or neuronal, cognitive or affective or motor or behavioral function/s or feature/s or diagnosis or evaluation of any brain related disease/s or impairment/s or dysfunction/s or alternatively any normal function/s or cognitive or affective or behavioral or motor or physiological or neuronal or electrical or electromagnetic or electrophysiological or neurophysiologic or other brain or neuron or neuronal or nervous system function/s pattern/s or capability/ies or operation/s etc., and which can furthermore determine or compute the precise value/s or intensity or duration or any other stimulation parameter/s which may involve any electrical or electromagnetic or neuronal or neural or cognitive or affective stimulation of any brain region/s or neuron/s or cognitive or affective or behavioral other brain function/s. Note that there is a two-way interaction between the BRAIN SENSORY-ENHANCEMENT COMPUTATIONAL MODULE 1102 and the BRAIN DIAGNOSTIC MODULE 1104 due to the fact that certain BRAIN SENSORY-ENHANCEMENT COMPUTATIONAL MODULE 1102 involve stimulation of single or multiple brain region/s or cognitive or affective or motor or other brain or neural or neuronal or other brain function/s or correlate/s etc. which can interact with—or be followed by- or be a part of the computational or algorithmic or analytical processing of the BRAIN SENSORY-ENHANCEMENT COMPUTATIONAL MODULE 1102 (e.g., for instance through the use of electrical or electromagnetic stimulation of a particular diseased or to be enhanced brain region/s or of an investigated normal brain or cognitive or affective or motor or behavioral brain function/s—for instance in an excitatory or inhibitory mode/s as a partial or full means for determining the specific interpretation or meaning or significance or function/s or role/s etc. of any particular brain or neuron/s or neuronal pattern or electrophysiological or neurophysiologic or electrical or electromagnetic pattern or value or event/s or occurrence/s or cognitive or behavioral or motor or intellectual function/s or capacity or level/s etc. The single or multiple computational processing, function/s or output of the BRAIN ENHANCEMENT COMPUTATIONAL MODULE 1102 (which are further described in details in the below Flow Chart FIG. 2) is outputted to either the BRAIN DIAGNOSTIC MODULE 1104 and/or to the BRAIN STIMULATION MODULE 1106. The BRAIN DIAGNOSTIC MODULE 1104 is capable of diagnosing or identifying any particular single or multiple brain disease/s or impaired brain functioning or diseased brain region/s or any single or multiple cognitive or affective or behavioral or neural or neuronal state/s or feature/s or condition or function/s and is also capable of computing whether or not any such brain region/s or neuron/s or neural network/s or neural activity, activation, pattern or cognitive or behavioral or neurophysiologic or electrical or electromagnetic or electrophysiological or other brain feature/s or correlate/s is likely to represent a normal or abnormal or above-normal characterization. Note that for certain applications the system or method described may end at the BRAIN DIAGNOSTIC MODULE 104 with the output of a diagnostic information (which may or may not interface with a physician or other appropriately trained professional or may be used for different applications including: brain cipher of interpreting or reading a person's thoughts, intentions, criminal or non-criminal, truthful or untruthful etc. or for diagnosing the existence of non-existence of any specific brain or mental or intellectual or behavioral or affective or neuronal disease/s or illness/es or condition or abnormality etc., or it may serve as the output for the BRAIN STIMULATION MODULE 1106 (e.g., including for the purpose/s therapeutic stimulation for single or multiple brain related disease/s or impairment/s or abnormality/ies or cognitive or affective or neuronal or motor impairment/s or for the enhancement of brain, neural, cognitive, affective, behavioral or motor or intellectual or other function/s etc. Finally, the BRAIN STIMULATION MODULE 1106 receives input from the BRAIN DIAGNOSTIC MODULE 1104 and is capable of determining the precise stimulation parameters (including but not limited to the intensity, duration, localization of single or multiple brain stimulation site/s or region/s or point/s etc.) that are necessary in order either improve any diseased or impaired brain or neural functioning or condition/s or state/s or to enhance any normal or sub-normal or above-normal brain or cognitive or behavioral or affective or neural or neuronal functioning etc. The BRAIN STIMULATION MODULE 1106 stimulates the originally measured BRAIN DISEASE OR ENHANCEMENT 1100 which referred to any single or multiple brain region/s or point/s or points or any brain-related feature/s or cognitive or affective or motor or electrophysiological etc. characteristics or condition/s which were measured or detected by the BRAIN ENHANCEMENT COMPUTATIONAL MODULE 1102 (as representing any normal or sub-normal or above-normal cognitive or affective or physiological or neuronal functioning or activity or disease/s or feature/s or deficient or abnormal trait or any such brain or neural or neuron's/s characteristic/s to be enhanced or improved or optimized or healed etc. Note also that the whole system or method or medical device or electrode or diagnostic system described can function as a complete system which comprises the BRAIN DISEASE OR ENHANCEMENT 1100 diseased or normal or to be enhanced or other regions, of cognitive or affective or motor or behavioral function/s or region/s or pattern/s plus the three other abovementioned modules which can also function as a continuous or intermittent or fixed or adjustable or other sensory-stimulation (automatic, semi-automatic, corrective, physician aided or technician aided or other input based system) which can continuously or progressively or corrective function that allows for a gradual or immediate or long-or short term method or system or medical device or electrode based method for combating or improving or healing any single or multiple brain related disease/s or function/s or impairment/s or for obtaining a (set or adjustable or dynamic) health or diseased state improvement threshold/s; Such an (open or closed) brain sensing & corrective stimulation loop may embody a full scale medical device/s for sensing or diagnosing or stimulating diseased brain regions or single or multiple brain diseases or for enhancing impaired or normal brain or cognitive or affective or behavioral functioning or for deciphering any single or multiple brain or neural activity or pattern/s or any spatial- or temporal- or computational processing, or may be embodied or encapsulated within a single or multiple brain or neural electrode/s which may allow for an automatic or semi-automatic or physician-aided stimulation of diseased brain region/s or cognitive or affective or behavioral or motor or other physiological feature/s or disease/s.

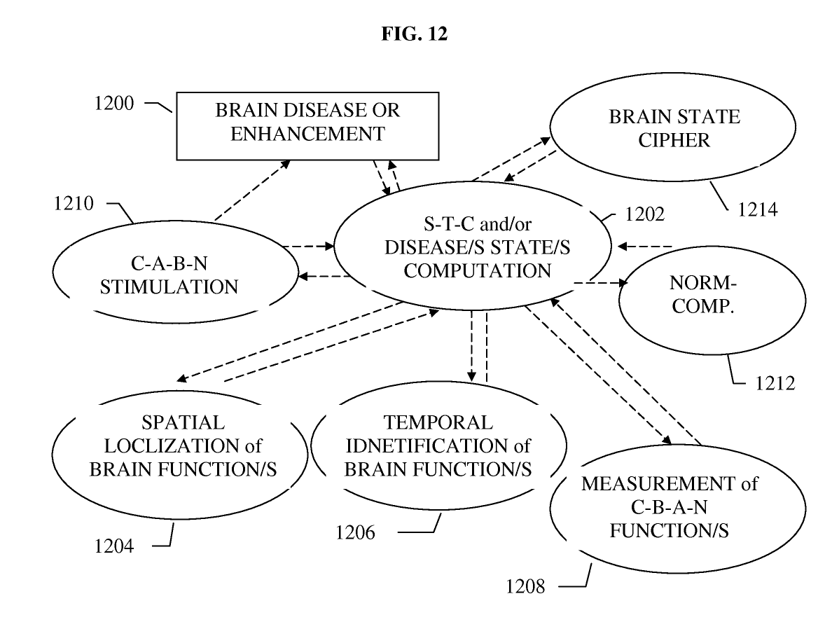

Reference is now made to FIG. 12 which describes a flow-chart diagram delineating the computational or algorithm or processing or analysis of computation/s taking place within the BRAIN SENSORY-ENHANCEMENT COMPUTATIONAL MODULE 1102 which also include input/s- and output/s- to- and from the BRAIN DISEASE ENHANCEMENT 1100 and input/s or output/s to- and from- the BRAIN STIMULATION MODULE 1106 (described in FIG. 11). The input received from the BRAIN DISEASE or ENHANCEMENT 1100 to the BRAIN SENSORY-ENHANCEMENT COMPUTATIONAL MODULE 1102 includes clinical or medical or psychiatric or psychological or professional information received from any other clinical, medical, care-taker or provider or by the individual or any of his legal or medical or professional representatives regarding any symptoms, any previous diagnosis or diagnoses or evaluation/s; Specifically, the input from the BRAIN SENSORY-ENHANCEMENT COMPUTATIONAL MODULE 1102 is outputted to the S-T-C and/or DISEASE/S STATE/S COMPUTATION 1202 which refers to the Spatial-Temporal-Computational processing, computation or algorithm carried out by this module and is capable of deciphering any single or multiple localized or dispersed or at single or multiple synchronized or un-synchronized spatial- temporal- or spatial-temporal points or region/s in the brain or any single or multiple cognitive, behavioral or computational or motor activity or activation of function/s or operation/s etc. Hence, the S-T-C and/or DISEASE/S STATE/S COMPUTATION 1202 obtains information regarding the specific brain or neural or neuronal or brain-related or any cognitive or affective or behavioral or physiological or neurophysiologic or even any body-related disease which is controlled or associated with any brain impairment/s or abnormality disease/s or impairment/s or abnormality or any neural or neuronal activity or activities or cognitive or affective or motor or physiological or other brain or neural or neurological or psychiatric related or associated pattern/s which is of interest or which is targeted for monitoring or for improvement/s of for enhancement or for decoding or for deciphering or for analysis etc. and based on this information or input from the BRAIN DISEASE OR ENHANCEMENT 1200 the S-T-C and/or DISEASE/S STATE/S COMPUTATION 1202 determines or controls or initiates or operates or collects data from any brain or neural or neurophysiologic or electrophysiological or cognitive or behavioral or motor or affective of other methodology which measures a SPATIAL LOCALIZATION of BRAIN FUNCTION/S 1204 or a TEMPORAL IDENTIFICATION of BRAIN FUNCTION/S 1206 or a MEASUREMENT OF C-B-A-N FUNCTION/S 1208 (e.g., Cognitive-Behavioral-Affective-Neuronal or Neuron/s Functions) or a NORM-COMP. 1212 (e.g., Norm based Computation); For all of these different types of information or computation or input there is potentially a two-way interaction between the S-T-C and/or DISEASE/S STATE/S COMPUTATION 1202 and each of these modules (e.g., #1204 through #1212). Thus, for instance, the S-T-C and/or DISEASE/S STATE/S COMPUTATION 1202 initiates, controls or determine which SPATIAL LOCALIZATION of BRAIN FUNCTION/S 1204 information or input needs to be received for a particular disease/s or brain or neural or neuronal impairment/s or abnormality/ies or for any particular brain or neural or neuronal function/s (e.g., including but not limited to any neural or neuronal or cognitive or behavioral or motor or any other brain or cognitive or affective or motor or neurophysiologic or behavioral activity/ies, function/s, operation/s etc. which may be desired or targeted to be deciphered or interpreted or to be enhanced or therapeutically treated etc.). Once the S-T-C and/or DISEASE/S STATE/S COMPUTATION 1202 gives the order or controls or initiates or outputs to the SPATIAL LOCALIZATION of BRAIN FUNCTION/S 1204 (or for that matter to any of the other above mentioned modules #1204 through #1212, together or separately in any possible combination/s) each of these module/s (separately or conjointly, synchronized, timed or unsynchronized or unpaired) collects the relevant information or measurement/s and transfers it or them to the S-T-C and/or DISEASE/S STATE/S COMPUTATION 1202 which allows the S-T-C and/or DISEASE/S STATE/S COMPUTATION 1202 to determine the precise interpretation or deciphering or analysis or discovery of the precise cognitive, behavioral, affective, motor or intellectual or neurophysiologic or operation or input or output or semantic or syntactic or associative or response correlate/s or significance or interpretation/s or meaning/s or function/s etc. The SPATIAL LOCALIZATION of BRAIN FUNCTION/S 1204 is responsible for collecting or measuring any brain or neural or neuronal or neurological or psychiatric or neuroscientific or cognitive or behavioral or affective or motor or any other neurophysiologic measure/s or activation/s or activity/ies or function/s or operation/s or trait/s or behavior/s or response/s or correlate/s etc. taken place or being measured at any single or multiple spatial point/s or pixel/s or neuron/s or neuronal or neural network or point/s within any neuron/s within the central nervous system or any single or multiple point/s, region/s, tissue/s, neuron/s etc.—at or across or measured across single or multiple temporal point/s or interval/s or duration/s etc. (which may include the localization of any brain or neural or neuronal any electrical or electrophysiological or neurophysiologic or electromagnetic or cognitive or behavioral or affective or motor or intellectual etc.). Hence, for any specifically identified brain disease/s or brain or neural abnormality or condition (e.g., identified by the BRAIN DISEASE OR ENHANCEMENT 1200) or for any other targeted brain-related or neuronal or cognitive or affective or behavioral trait/s or activity/ies or function/s or operation/s which is targeted or wished for deciphering, interpretation, analysis or manipulation or improvement/s or enhancement/s or the SPATIAL LOCALIZATION of BRAIN FUNCTION/S 1204 collects any specific or general measurement/s or data related to the precise single or multiple spatial point/s or region/s or neuron/s or neural network/s or locus or loci in the brain or any other part of the nervous system (e.g., at any single or multiple continuous or intermittent, synchronized or un-synchronized with any other brain measurement/s or spatial- or temporal-measurement/s) relating or associated or linked or caused by or causing or associated with or connected with any cognitive or behavioral or affective or motor or any other brain-related or neural activity/ies or function/s or operation/s, including also the spatial-temporal- or spatial-temporal pattern/s or activity or activation or resting-state or active-state or computation or processing or algorithm-based computation of the brain or any specific neuron/s or region/s within the brain. This information measured or collected by the SPATIAL LOCALIZATION of BRAIN FUNCTION/S 1204 is outputted to the S-T-C and/or DISEASE/S STATE/S COMPUTATION 1202 and may also interact with- or direct- or assist in synchronizing by the S-T-C and/or DISEASE/S STATE/S COMPUTATION 1202 its control or computation or determination or collection or input- or output- or any interaction with any of the other modules #1206 through #1212 (separately or independently or conjointly). This is also closely related to a ‘Functional Specificity Principle’ discovered and claimed in this patent application which refers to the idea that any brain or neural ciphering or interpretation or decoding or analysis relating to discovering its underlying or associated or caused or linked function or operation is claimed to be specific in its function or operation or response or input or significance to a specific spatial (single or multiple) localization and/or temporal point/s; In fact, one of the key principles of operation in this patent is the notion that in order to discover or identify or interpret or accurately decipher or manipulate or indeed replicate or regenerate or impact or influence or therapeutically heal or improve or positively affect—the neural or neuronal code/s or brain code of any cognitive or behavioral or affective or motor or any neurophysiologic or electrophysiological activity/ies or function/s or operation/s it is necessary to precisely identify the spatial- and/or temporal- and/or electrical and/or electromagnetic and/or electrophysiological and/or computational-pattern/s associated or correlated or causally related or producing or produced or associated with any such specific neural- or neuronal- or cognitive- or behavioral- or affective- or motor- or neurophysiologic activity/ies or activation or function/s or operation/s etc. A further key tenet of this patent application is that once the spatial- and/or temporal- and/or electrophysiological- and/or computational- and/or cognitive- and/or affective- and/or motor- and/or neurophysiologic pattern/s has been identified it can be broken-down or analyzed or interpreted or deciphered or computed or accurately decoded in order to determine the precise significance/s or operation/s or meaning/s or function/s or input/s- or output/s- or functional significance or operation/s or role/s; A further tenet/claim of this patent application is that this functional and/or operational and/or spatial- and/or temporal- and/or spatial-temporal and/or algorithm/ic and/or computational specificity is universal in the sense that it serves as the basic template for decoding or encoding or representing any such specific cognitive or behavioral or affective or motor or neurophysiologic or electrophysiological or electrical or electromagnetic pattern/s or characteristic/s or shape or spatial- and/or temporal and/or spatial-temporal and/or algorithm/ic and/or electrophysiological and/or electrical and/or electromagnetic pattern/s or value/s or feature/s is shared by all or most or a large number or a portion of all individuals (e.g., generally or specifically for a group of same-language/s or same-region/s or same gender-race-geographical location/s or other cultural or linguistic or other characteristic/s); Finally, another key claim of the present invention is that the identification of such functional and/or operational and/or spatial- and/or temporal- and/or spatial-temporal and/or algorithm/ic and/or computational specificity may allow for its replication or regeneration or therapy or improvement/s in impaired function/s or disease/s or diseased brain or neural region/s or cognitive or affective or behavioral or motor or neurophysiologic function/s—through the manipulation of such electrophysiological or electrical or neurological or neuronal or neural specific activation or activity/ies at the specific spatial- and/or temporal- and/or spatial-temporal and/or electrophysiological localization and/or pattern/s and/or intensity and/or frequency and/or time- or duration- and/or associated with a specific cognitive or affective or behavioral or motor or neural activity or function/s or operation/s etc.; and moreover that the usage and/or manipulation and/or amplification and/or alteration in terms of duration and/or intensity and/or frequency and/or spatial- and/or spatial-temporal or temporal localization may allow for the regeneration of lost cognitive or affective or neuronal or motor or any other neurophysiologic or electrophysiological function/s or operation/s etc.—including its replication or the replication of a normal representation/s or pattern/s of the healthy or normal or above-normal brain or neural or cognitive or affective or behavioral or neurophysiologic function or operation of representation at the same specific spatial- and/or temporal and/or spatial-temporal and/or electrophysiological or electrical or neurophysiologic and/or algorithm and/or computation.

Hence, the S-T-C and/or DISEASE/S STATE/S COMPUTATION 1202 receives input or measurement/s or clinical evaluation/s or any other cognitive or affective or behavioral or neural/neuronal input or data from the BRAIN DISEASE OR ENHANCEMENT 1200 regarding the particular brain- or neural- or neuronal-disease/s or diseased region/s or diseased state/s or regarding any particular singular or multiple cognitive or affective or behavioral or neuronal etc. trait/s or value/s or brain or neural region/s or neural network/s which is targeted or wished to be enhanced or deciphered etc. Based on this input/s the S-T-C and/or DISEASE/S STATE/S COMPUTATION 1202 directs or outputs or interfaces with any single or multiple of these module/s: the SPATIAL LOCALIZATION of BRAIN FUNCTION/S 1204 which is capable of measuring or detecting any spatial- and or temporal or spatial-temporal brain or neuronal or neural activity/ties or pattern/s occurring at any single or multiple spatial/temporal point/s or region/s or associated with any cognitive or affective or any brain- or neural related function/s (including specifically those brain region/s or function/s that the S-T-C and/or DISEASE/S STATE/S COMPUTATION 1202 has instructed or outputted etc.), or DISEASE/S STATE/S COMPUTATION 1202 directs or outputs or interfaces with the TEMPORAL IDENTIFICATION of BRAIN FUNCTION/S 1206 which is capable of identifying the temporal or spatial or temporal spatial configuration/s or pattern/s or shape or temporal or spatial-temporal pattern or dynamics—including the S-T-C and/or DISEASE/S STATE/S COMPUTATION 1202 interactive or interaction or interface/s or computation/s (including in real-time or off-line or otherwise) with any of the other modules including (but not limited to) the capacity to dissect or analyze or compute or determine the segment/s or segmentation or correlation/s or association of any other mathematical computation of the relationship/s between the spatial- or temporal- or spatial-temporal and/or intensity and/or duration and/or threshold/s and/or dynamic/s and/or relationship/s between any cognitive/s or behavioral/s or affective/s or intellectual/s or neurophysiologic or electrophysiological or electrical or electromagnetic or any other brain- or neural- or neuronal-activity/ies and/or function/s and/or pattern/s etc. and any correlated cognitive or affective or behavioral or input/s or output/s or brain function/s etc., or DISEASE/S STATE/S COMPUTATION 1202 directs or outputs or interfaces with the MEASUREMENT of C-B-A-N FUNCTION/S 1208 which is capable of measuring or detecting or otherwise analyzing or deciphering any cognitive or behavioral or affective or neurophysiologic or neurological or neural or neuronal activity or activities or pattern/s or function/s or localization/s or correlate/s or other feature/s including any interaction of these single- or multiple-brain region/s or brain function/s or neural function/s, activity/ies or feature/s including any interaction/s or relationship/s with any of the other modules (#1200, #1202 through #1212) and/or deciphering of the meaning/s or function/s or correlate/s or association/s or significance/s or operation/s or function/s or input- or output of any of these neurophysiologic, electrophysiological, cognitive or affective or neural or neurological or neural network/s. or DISEASE/S STATE/S COMPUTATION 1202 directs or outputs or interfaces with the C-A-B-N STIMULATION 1210 which is capable of cognitive or behavioral or affective or neural or neuronal or neuron/s or neural network/s or neurological or any other brain-related or neuronal-related stimulation/s (e.g., at any single or multiple point/s, region/s, domain/s, feature/s or function/s or interval/s or intensity/ties or frequency/ies or dynamic/s or pattern/s etc.)—including but not limited to its interface/s or interaction/s with any single or multiple or any possible combination of the other module/s #1200 and/or #1202 through #1212. The C-A-B-N STIMULATION 1210 module produces any single or multiple neuron/s or neuronal or neural network or any brain-related stimulation/s or any cognitive or affective or behavioral or neurophysiologic or electrophysiological or other brain- or neuron/s- or neural-stimulation including any spatial-temporal or spatial-temporal pattern/s or interval/s or intensity/ies or duration/s or dynamic/s or interactive-stimulation of any single or multiple neuron/s or region/s or brain or cognitive or affective or motor function/s which can also include any real-time or off-line or constant or adjustable or continuous or intermittent or haphazard or any other pattern/s of stimulation or any other spatial-temporal- or spatial-temporal stimulation/s schedule/s or pattern/s or paradigm/s or program/s, which may also be timed- or synchronized- or configured- or coordinated- with either any single or multiple neural- or neuronal- or brain activity (e.g., including at any point/s or region/s or modality or neuron/s or neuronal or neural network/s or cognitive or behavioral or affective or motor stimulation/s), and which may also be synchronized with any of the data or patter/s or norm/s or function/s or input/s or output/s or operation/s found within any of the module/s #100 and/or #202 through #212; The system or method or medical device or apparatus described generally in this invention or in any sub-components or module/s or function/s or operation/s or any combination/s of any of the above mentioned modules—also includes the possibility of the C-A-B-N STIMULATION 1210 to allow for the operation of ‘smart-electrode/s’ which are capable of measuring (e.g., including in real-time or off-line or in any other spatial-temporal or spatial-temporal configuration any diseased or normal or targeted or to be measured or enhanced or modified brain region/s or point/s or point/s or any brain- or neural- or neuronal-pattern/s or function/s or timing or synchronization/s etc.) or stimulating any single or multiple brain region/s or point/s and/or cognitive and/or affective and/or behavioral and/or motor and/or neurophysiologic and/or electrophysiological pattern/s and/or spatial- and/or temporal and/or spatial-temporal point/s or pattern/s or activity or activities, which may bring about an alteration of the baseline or diseased or normal or targeted neural or neuronal activity/ies or cognitive or affective or behavioral or motor function/s or operation/s or spatial-temporal pattern/s or dynamic/s etc., and/or which may also include a synchronized or closed- or open-control loop (e.g., including a predetermined criteria or an interactive or a real-time or an intermittent feedback loop etc.). The C-A-B-N STIMULATION 1210 module and its interaction with any of the other module/s allows for a system or method or medical device or function/s or apparatus/es which can measure any targeted single or multiple brain region/s or diseased brain region/s or to be enhanced or optimized brain region/s or point/s or locus or loci and/or cognitive or behavioral or affective or motor or electrophysiological or neurophysiologic feature/s or correlate/s or value/s—including (but not limited) to a system or method or medical device or electrode/s or ‘smart electrode/s’ which are capable of measuring any baseline or activity/ies or pattern/s or characteristic/s or any single or multiple and/or spatial- and/or temporal brain region/s or diseased brain region/s or locus or loci or spatial- or temporal- or spatial-temporal and/or determining any desired or targeted criteria or criterion or threshold/s or value/s or determining any type/s or pattern/s or control-loop/s and/or modulation/s of any targeted or identified or measured or diseased or to be enhanced or modulated or monitored single or multiple brain region/s or neural or neuron/s activity/ies or pattern/s or feature/s or characteristic/s. The C-A-B-N STIMULATION 1210 also can allow for a recording and/or measurement/s and/or stimulation/s and/or presentation/s of any stimulus or stimuli (including but not limited to cognitive or affective or behavioral or neural or neuronal or neurophysiologic or electrophysiological or other brain related stimulation) pre- or post- or during- any brain activity or activities or at baseline and/or at diseased and/or to be enhanced brain region/s neural or neuronal or cognitive or behavioral or affective or motor or electrophysiological or neurophysiologic or other brain related activity or at baseline or targeted towards any deficient or abnormal or dysfunctional brain or neural or neuronal activity or pattern/s or etc. and may also allow for the continuous or constant or intermittent or criteria-based or improvement based or threshold based stimulation/s of diseased brain region/s or single or multiple diseased or impaired or dysfunctional or to be improved or enhanced brain region/s or function/s or spatial- or temporal or spatial-temporal locus or loci or pattern/s or dynamic/s or feature/s etc. Finally, the DISEASE/S STATE/S COMPUTATION 1202 directs or outputs or interfaces with the NORM-COMP. 1212 which is capable of comparing any whole or partial or segment/s or pattern/s or dynamic/s and/or combination/s or association/s or correlate/s of any of the other module/s (e.g., #1200, #1202 through #1210) with predetermined or measured or computed or other health or diseased or criteria based norms of cognitive or behavioral or affective norms or any other brain or neuron/s or neural-network/s or disease/s or abnormal or healthy or enhanced or above-normal functioning or level/s or threshold/s or intensity/ies norm/s or comparison/s or single or computation or ability to determine whether a particular measured neuron/s or neuronal or brain related or cognitive or affective or intellectual or motor activity or activities or pattern/s or intensity/ies or shape or spatial- or temporal or spatial-temporal pattern/s or activity/ies o frequency or location or association/s with any cognitive or affective or motor or neurophysiologic or electrophysiological or neural or other brain activity/ies is likely to be normal or abnormal or impaired or dysfunctional or above-normal or modulated and/or in what manner or through what stimulation protocol/s or parameter/s it is possible to alter or improve or normalize or detect or positively change such electrophysiological or neurophysiologic or cognitive or affective or motor or intellectual or electrophysiological or neuron/s or neuronal or brain related operation/s or pattern/s or activation/s or functioning etc.

Based on these single or multiple computation/s or processing/s or interaction/s between the BRAIN DISEASE OR ENHANCEMENT 100 and any single or multiple other modules #1210 through #1212 the S-T-C and/or DISEASE/S STATE/S COMPUTATION 1202 is capable determining or computing any single or multiple brain disease/s or brain disease/s state/s or condition/s or impairment/s or abnormality/ies or any single or multiple brain region/s which exhibit sub-normal or abnormal or normal or enhanced brain- or neuronal- or neural- or neural-network- or cognitive- or affective-state or operation or functioning or baseline or response or input or output, or decipher the significance or meaning/s or operation/s or function/s or interpretation/s or correlate/s or cause/s or effect/s or association/s with any of the single or multiple spatial-temporal brain region/s or occurrence/s or event/s or phenomena or activity/ies or diagnosis or diagnoses of any single or multiple brain disease/s or brain illness/es or abnormal or impaired or dysfunctional activity/ies, or determine what is the precise intensity and/or magnitude and/or stimulation parameter/s and/or stimulation single or multiple region/s or point/s or neuron/s or neural-network/s etc. necessary in order to bring about an improvement or an enhancement or a positive change/s and/or a healing and/or obtaining or attainment of a certain criteria or criterion based improvement or enhancement/s or modulation in any single or multiple brain or neural or region/s or spatial-temporal or cognitive or behavioral or affective pattern/s or intensity or criterion/criteria or functioning or performance or operation/s or deciphering of the meaning of interpretation or role/s or function/s or input/s or output/s of any such brain or neural or neuron/s function/s or activity/ies or state/s or enhancement/s etc. Indeed, the BRAIN STATE CIPHER 1214 is outputted from the BRAIN SENSORY—ENHANCEMENT COMPUTATIONAL MODULE 1202 (e.g., based on the S-T-C and/or DISEASE/S STATE/S COMPUTATION 1202 computation of the BRAIN STATE CIPHER 1214) to the BRAIN STIMULATION MODULE 1206 (depicted in FIG. 1), where it assists in determining the precise stimulation parameter/s of the BRAIN STIMULATION MODULE 1206 of single or multiple BRAIN DISEASE or ENHANCEMENT 1200 region/s or function/s or cognitive or affective or neuronal or neurophysiologic or electrophysiological region/s or feature/s or trait/s or function/s or operation/s etc. Note then that the system or method or medical device described in this invention and also depicted in FIGS. 11 & 12 allows for both a diagnosis and/or evaluation or deciphering of any brain or neural or neuronal activity or activities or cognitive or affective or neuronal or neural pattern/s or activity/ies or function/s or operation/s or characteristic/s and/or any brain disease/s or impaired or dysfunctional or dysfunctional and/or deficient and/or to be improved and/or enhanced brain or cognitive or affective or behavioral or motor function/s and/or any neural activity/ies or function/s or input/s or output/s or deciphering of any single or multiple brain region/s and/or spatial- and/or temporal- and/or spatial-temporal activity or activities or function/s or operation/s or interpretation/s or role/s or significance/s, and/or which is capable of altering or modulating or healing or positively affecting or changing any single or multiple diseased state/s or condition/s or region/s or function/s and/or enhancing or optimizing or improving any brain or neural or cognitive or neuronal or motor or any other neurophysiological or electrophysiological function/s or operation/s.

Thus, for instance, the method or system or medical device or apparatus or electrode/s or smart electrode/s or mechanism/s described in this invention include also the development of a smart electrode or smart stimulation system or method or medical device which can be utilized for any brain disease/s or neural or neuronal or brain disease/s treatment or diagnosis or modulation of for enhancement of any normal or subnormal or above-normal brain or cognitive or affective or intellectual or motor or neurophysiologic or electrophysiological function/s or operation/s—including for instance a smart electrode/s or system/s or medical device for treating Parkinson's disease;

First, an embodiment of one possible deciphering or detection and/or interpretation and/or translation and/or determination and/or manipulation and/or modulation/s of any brain or neural or any single or multiple spatial and/or temporal and/or spatial-temporal brain or neural or neuronal or neurological or cognitive or affective or motor activity/ies or pattern/s or function/s or region/s or deciphering and/or manipulation of any electrophysiological activity/ies or activation/s and/or pattern/s and/or diseased or impaired or abnormal or deficient or dysfunctional or above-below- or normal-brain or neural or cognitive or affective or motor or behavioral function/s or role/s and/or the capacity to alter or modify or heal or improve function/s or functioning or disease/s or disease-state/s and/or improve sub-normal or even normal or above-normal functioning or level/s or attaining any (predetermined, computed, adjustable, open or closed loop controlled criteria for health or diseased improvement etc.) One such possible embodiment relates to the monitoring or measurement/s or analysis/es of for instance Parkinson's disease related sub cortical or cortical diseased region/s such as the Substantia Nigra or motor or visual or Amygdala or other impaired regions—which will constitute clinical or neurophysiologic or electrophysiological or cognitive or motor or behavioral or clinical or other medical, neurological or neuroimaging or EEG or ERP or cognitive or behavioral or neuropsychological or neurological measures relating to any index or indices of Parkinson's disease which may be measured at any single or multiple brain region/s or point/s or function/s or cognitive or affective or motor or cognitive feature/s or level/s or threshold/s or criterion/criteria etc. and be measured as part of the BRAIN DISEASE or ENHANCEMENT 1200 module which will be transferred to the BRAIN SENSORY-ENHANCEMENT COMPUTATIONAL MODULE 1202; In terms of one of the possible embodiment/s or this invention the BRAIN DISEASE or ENHANCEMENT 1200 may be embodied within a single or multiple sensing device/s or electrode/s or electrophysiological or cognitive or affective or motor or intellectual or behavioral or neurophysiologic or correlate/s measures included within the BRAIN SENSORY-ENHANCEMENT COMPUTATIONAL MODULE 1202 (including continuously, intermittently or at any real-time or off-line or other spatial- and/or temporal and/or spatial-temporal point/s or region/s or neuron/s by any invasive or non-invasive single or multiple brain or neural or neuronal measurement/s—which may (or may not) be accompanied or associated either in the same or different single or multiple brain region/s or point/s or locus or loci stimulation electrode/s or other electrical or electrophysiological or electromagnetic or neuronal or cognitive or affective or electrophysiological stimulation modality/ies included within the BRAIN STIMULATION MODULE 1206, which may include or be embodied within the same electrode/s or sensing and/or stimulating mechanism or region/s or modality/ies (e.g., such as for instance a single or multiple electrode/s placed at the same single or multiple brain region/s or point/s or neuron/s or the same or utilizing the same or different single or multiple cognitive or affective or behavioral or motor modality/ies or region/s or neuron/s or neural-network/s etc., and which may be accompanied by same-time or real-time or different or same or different or single or multiple spatial- and/or temporal and/or spatial-temporal stimulation/s (relative to—or as- the sensing or sensory spatial- and/or temporal and/or spatial-temporal functioning or measurement/s or computation/s or input or output etc.), and which may further be accompanied or function with or without an open- or closed- or without any control loop (as described by the closed open or closed loop in the delineation of FIG. 11's depiction of a potential loop formed between the BRAIN DISEASE or ENHANCEMENT 1200, BRAIN SENSORY-ENHANCEMENT COMPUTATIONAL MODULE 1202, BRAIN DIAGNOSTIC MODULE 1204, BRAIN STIMULATION MODULE 1206, and back to the BRAIN DISEASE or ENHANCEMENT 1200 and potentially again through the same loop in real-time, or off-line or continuously or intermittently etc.; Obviously, such an open- or closed-computational/control loop is supported by the flowchart in FIG. 12 of the computation/s or process/es taking place within the BRAIN SENSORY-ENHANCEMENT COMPUTATIONAL MODULE 1102 which includes the possibility of continuous or discontinuous or intermittent interface/s or interaction/s or single or multiple input/s and/or output/s between the S-T-C and/or DISEASE/S STATE/S COMPUTATION 1202 and the BRAIN DISEASE OR ENHANCEMENT 1200 and/or BRAIN STATE CIPHER 1214 output which is outputted to the BRAIN DIAGNOSTIC MODULE or directly to the BRAIN STIMULATION MODULE 1206 (which then is applied to the BRAIN DISEASE or ENHANCEMENT 1200), which is also obviously supported by the single or multiple interaction/s or interface/s or input/s or output/s between the S-T-C and/or DISEASE/S STATE/S COMPUTATION 1202 and any single or multiple other modules #1204 through 1212 and/or at any spatial- and/or temporal and or spatial-temporal point/s or interval or schedule/s etc.

Hence, one possible embodiment of the present invention include/s a smart electrode for treating Parkinson's disease which is based on the BRAIN DISEASE OR ENHANCEMENT 1200 measurement/s or determination or computation or analysis of any single or multiple brain region/s or locus or loci or cognitive of affective or behavioral trait/s or function/s or value/s or feature/s or abnormality/ies or indication or determination of the precise abnormality/ies or deficiencies that exist or are projected or predicted to appear in an individual suffering from Parkinson's disease (or any other brain related disease/s or condition/s or predicament/s or impairment/s or in an individual or a group whose cognitive or behavioral or affective or other brain related function/s are to be improved or enhanced etc.)—which is transferred or outputted to the BRAIN SENSORY-ENHANCEMENT COMPUTATIONAL MODULE 1102 which then can measure or compute or determine or analyze the precise deficiency/ies or abnormality/ies or impairment/s (e.g., in terms of any single or multiple associated or unassociated brain or neuron/s or neuronal or cognitive or affective or behavioral or other brain related impairment/s, deficiency/ies, abnormality/ies etc. or conversely for enhancing a specific brain or motor or intellectual or affective or neuron/s or neuronal function/s etc.) and/or compute the specific neural or neuronal or cognitive or affective or behavioral stimulation/s or input/s or modulation/s (including any single- or multiple-spatial-temporal or spatial-temporal or other stimulation/s or presentation/s or input/s) that are necessary in order to produce or induce a positive or therapeutic effect/s or regeneration of lost cognitive function/s or structure/s or neuron/s or connection/s etc. and/or the single or multiple stimulation/s or schedule/s or stimulation parameter/s or interval/s or intensity/ies of any other stimulation or neural or cognitive or behavioral or motor input or stimulation or modulation etc. The BRAIN SENSORY-ENHANCEMENT COMPUTATIONAL MODULE's determination/s or computation/s of the specific single or multiple spatial- or temporal or spatial-temporal abnormality/ies or neural or cognitive or behavioral or affective or motor or brain related abnormality/ies or deficiency/ies or impairment/s is carried out or achieved through the above mentioned interaction between any single or multiple of its flowchart (FIG. 2) modules at any single or multiple spatial- or temporal- or spatial-temporal point/s, interval/s or combination/s and may occur off-line or in real-time, or in any other spatial or temporal or spatial-temporal configuration/s and/or may occur within the implanted single or multiple electrode/s or within any other invasive or non-invasive mechanism or operation/s etc. constituting this invention as described above or in FIGS. 11 and 12.

Specifically, the operation of the BRAIN SENSORY-ENHANCEMENT COMPUTATIONAL MODULE 1102 in the case of Parkinson's disease (e.g., as an example for all other brain disease/s or condition/s etc.) may include an analysis by the S-T-C and/or DISEASE/S STATE/S COMPUTATION 1202 of the precise nature, and/or characteristic/s and/or feature/s and/or level/s and/or threshold/s and/or abnormality/ies and/or functional impairment/s and/or cognitive or behavioral and/or motor and/or affective impairment/s or abnormality/ies at any given spatial or spatial-temporal or temporal point/s or interval/s or combination/s or sequence/s or pattern/s etc. related to any impairment/s or abnormality associated with Parkinson's disease (or any other brain disease/s)—which may be computed by the S-T-C- and/or DISEASE/S STATE/S COMPUTATION 1202 based on- or through- any combination of any single or multiple modules (e.g., including #1200 and/or #1202 through #1214): Thus, for instance, the S-T-C- and/or DISEASE/S STATE/S COMPUTATION 1202 may interact or receive input or send output to the SPATIAL LOCALIZATION of BRAIN FUNCTION/S 1204 which may measure any cell/s or neuron/s or brain- or neural- or neuron/s or point/s or region/s or locus or loci or specific diseased (Parkinson's related) brain region/s or point/s or neuron/s or neural network/s etc. and/or in combination/s with the TEMPORAL IDENTIFICATION of BRAIN FUNCTION/S 1206 which can monitor or measure or identify any particular or specific or general or generalized or other temporal- or spatial- or spatial-temporal pattern/s of activity/ies or neural activity/ies or neural or brain or neuronal activation/s and/or cognitive or affective or motor or neurophysiologic or electrophysiological activation/s or value/s or pattern/s or threshold/s or framework/s or mold/s or structure/s or pattern/s or association/s with any specific or general spatial- or temporal or spatial-temporal or cognitive or affective or stimulus or stimuli presentation of cognitive processing or task/s of function/s or motor function/s or operation/s or input or output (e.g., such that for instance a particular motor function/s which may be impaired or diseased or abnormal or deficient or sub-normal or above normal which may be associated with Parkinson's disease (PD) or with any PD deficiency/ies, impairment/s or malfunction/s etc. may be identified or deciphered or properly interpreted or decoded into a particular single or multiple spatial-temporal- or spatial-temporal pattern/s or activity/ies of activation/s or specific electrophysiological activation/s etc.; Or that the potential interaction or interplay or interface or dynamics between the S-T-C- and/or DISEASE/S STATE/S COMPUTATION 1202 and any single or multiple modules including #1200 and/or #1202 through #1214 and/or any of these module/s input/s and/or output/s and/or processing/s or computation/s can determine whether any specific brain function/s or operation/s or pattern/s or any single or multiple spatial- or temporal or spatial-temporal activity/ies or activation/s is likely to be abnormal or is computed to be impaired or abnormal and/or whether any such single or multiple spatial- or temporal or spatial-temporal pattern/s may be above—or below- or at- any single or multiple normal brain or neuron/s or neural or cognitive or affective or motor or behavioral or neurophysiologic or electrophysiological or intellectual or any other brain- or neural-functioning and/or what may be the necessary electrical or electromagnetic or electrophysiological or cognitive or affective or motor or behavioral or intellectual or other neuronal or brain stimulation/s that may be necessary to improve or enhance or therapeutically treat or heal or improve or bring the neuronal or brain or cognitive or affective or motor functioning back to normal or subnormal or even to a specific threshold/s or value/s or level of spatial- or temporal- or spatial-temporal optimized or improved or normal or above normal functioning or state/s or condition/s or operation/s etc. or improvement in any single or multiple specific or general identified PD abnormality or neural abnormal or cognitive or affective or motor or neurophysiologic abnormal or deficient or impaired functioning etc.; One possible embodiment/s of the current invention may be the convergence or cross-talk or interface or computation/s by the S-T-C and/or DISEASE/S STATE/S COMPUTATION 1202 of the identification of a specific Parkinson's related or impaired spatial- or temporal- or spatial-temporal neural or brain or neuronal or neural-network/s region/s or function/s or cognitive or behavioral or affective or motor or intellectual abnormal or abnormality or deficient or impaired functioning or region/s or characteristic/s and/or identification/s or measurement/s or computation/s of the spatial- or temporal- or spatial-temporal pattern/s or neural or neuron/s activity/ies or value/s or threshold/s or intensity/ies or frequency/ies or characteristic/s or feature/s—which can be derived through a combination of any single or multiple modules (including #1202 through #1214) including for instance any combination of single or multiple analysis/es or computation/s within-between-in convergence- or through interaction between- any of these single of multiple (above mentioned) modules that can identify or measure or compute the spatial-temporal or spatial temporal of any Parkinson's (or any other brain disease or abnormal or subnormal or above normal or to be enhanced medical or neural or psychological or psychiatric or neurophysiologic or cognitive or affective or motor or intellectual etc. function/s or functioning or role/s or state/s) related cognitive or affective or behavioral or neural or neuronal or brain function/s or functioning—which can be gained through the comparison of any single of multiple cognitive or affective or motor or intellectual or neuronal or other brain functioning in a PD patient/s with established norm/s for healthy or sub-normal or above normal or normal functioning or neuron/s or cognition or affect or behavior etc. such as by the NORM-COMP. 1212 module with- or without interaction/s with the MEASUREMENT of C-B-A-N FUNCTION/S 1208 with or without or in any combination/s with the C-A-B-N STIMULATION 1210 (which can assist in determining or measuring or arousing or invoking measurable response/s or processing/s of specific cognitive or affective or behavioral or neuronal task/s or function/s etc.) with or without interaction with the BRAIN STATE CIPHER 1214 (which can decipher or diagnose or evaluate or compute the existence of any specific motor or behavioral or cognitive or neuronal or interpret or decipher any such functioning or state/s—which in turn can interact with the NORM-COMP. 1212 module to determine whether or not a specific measured or computed or identified brain or neural or neuronal or any single or multiple spatial- or temporal- or spatial-temporal function/s or neurophysiologic or electrophysiological measurement/s or pattern/s or locus or loci or value/s of function/s or operation/s or performance/s etc. is normal or abnormal or above- or below- or at normal functioning or level/s or threshold/s etc.), and which may in turn interact with any other module including specifically with the S-T-C and/or DISEASE/S STATE/S COMPUTATION 1202 module to determine or compute any single or multiple and/or any spatial- or temporal- or spatial-temporal or cognitive or behavioral or affective or motor or neurophysiologic stimulation/s necessary in order to obtain a specific improvement/s or alteration/s or modulation/s or healing or treatment/s or improvement/s or optimization/s of the identified diseased brain or neural or neuronal functioning or healthy or improved or enhanced or healed brain or neural or neuron/s or cognitive of affective or motor or neurophysiologic functioning or state or improvement or healing of enhancement of any PD feature/s or state/s or function/s or functioning.

Indeed, there are numerous possible embodiment/s for the present invention/s including for the above outlined embodiment of the invention's system or method or medical device/s or apparatus/es capable of measuring or computing or deciphering or computing any single or multiple and/or spatial- and/or temporal- and/or spatial-temporal neural or neuron/s or neuronal and/or any cognitive or affective or behavioral or motor or neurophysiologic or other brain functioning/s and/or identifying or deciphering or measuring or computing any PD (or any other brain disease/s or impairment/s or condition/s or above- or below- or at-normal brain or neural or cognitive or affective or behavioral or motor or neurophysiologic or electromagnetic or electrical function/s or functioning) and/or for affecting change or improving functioning or any single or multiple brain or neural or neuron/s or cognitive or affective or motor or behavioral function/s or state/s or condition/s or disease/s or abnormality/ies etc.: Among these embodiment/s there is also a possibility of measuring invasively or non-invasively and/or in ‘real-time’ or ‘off-line’ and/or in- or at- any single or multiple spatial and/or temporal- and/or spatial-temporal and/or measuring and/or computing and/or analyzing and/or interpreting and/or deciphering any single or multiple electrophysiological or neurophysiologic or neural or neuron/s and/or cognitive and/or affective and/or motor and/or intellectual and/or any other brain or neural function/s or feature/s or characteristic/s etc.—which can be combined with any spatial-temporal neural or neuronal or cognitive or affective or behavioral or brain stimulation/s (including at any stimulation or intensity or stimulation parameter/s or schedule or time/s or interval/s or any single or multiple pattern/s or spatial or temporal or spatial-temporal locus or loci etc.) and/or in order to attain any improvement/s or enhancement/s or modulation/s or healing or optimization/s of normalization/s of any single or multiple PD (or any other brain disease/s or impairment/s or enhancement of normal- or sub-normal or above-normal brain functioning etc.); One such possible embodiment may be to measure with existent or proprietary or created EEG or ERP or any electrophysiological or neurophysiologic (invasive or non-invasive including wireless or other measuring or computing device/s or apparatus/es) etc.—in real-time or at any single or multiple and/or spatial- and/or temporal and/or spatial-temporal point/s or region/s and/or before and/or during- and/or after- any brain or neural or neuronal (including invasive or non-invasive) brain or neural or neuron/s stimulation and/or related to PD and/or to any PD related impairment/s or abnormality (e.g., including brain, neural, in or associated with any neuron/s or cognitive or affective or behavioral or neuronal abnormality/ies of impairment/s etc.) and/or combine- or interface- or interact- such brain or neural or any single or multiple spatial-temporal or any single or multiple PD related or abnormal or impaired brain activation or activity/ies or pattern/s etc. with any invasive or non-invasive electrode/s or smart electrode/s and/or cognitive or behavioral or affective or motor or neurophysiologic stimulation/s—including the possibility of synchronizing or timing or pairing between any such single or multiple and/or spatial-temporal stimulation combination/s and any concurrent and/or timed and/or synchronized (real-time or off-line or intermittent or continuous or discontinuous) electrophysiological or neurophysiologic or cognitive or affective or behavioral or motor etc. of diseased or impaired or abnormal of identified PD (or any other brain disease/s) functioning or malfunctioning or impairment/s or abnormal feature/s etc., including the possibility of a smart loop or of an open- or closed-loop or of an adjustment or modulation or personalization of personal medicine for the fine-tuning of optimization or normalization or healing of improvement or attainment of a specific (predetermined or interactively determined or otherwise determined or computed) threshold or norm or improvement of level/s or target level/s or feature/s etc.) is achieved or improved etc. Thus, for instance it is possible to embody a single or multiple electrode/s or smart electrode or sub-dermal or super-dermal (above skin or below or within skin or skull or neuron/s) placement/s or implantation/s of electrode/s or ‘smart electrodes’ or any other neural or electrical or electromagnetic or electrophysiological and/or any synchronized or unsynchronized convergent or non-convergent cognitive or behavioral or motor or behavioral stimulating element/s which are capable of stimulating either the same and/or other single or multiple spatial- or spatial-temporal or temporal-brain region/s or neuron/s or function/s or brain associated function/s or feature/s or cognitive or affective or motor or behavioral etc. function/s or capabilities or features of diseased feature/s etc.—including the possible synchronization of any of these single or multiple stimulation/s and/or at any single or multiple spatial-temporal or spatial or temporal point/s or region/s or brain or neural or cognitive or affective or motor etc. and/or any PD abnormal or diseased function/s etc.—including until a certain norm of any single or multiple brain related or cognition or behavioral or motor or neurophysiologic or other improvement/s or PD (or any other brain related disease/s or state/s or enhancement/s of normal or supernormal or subnormal brain or cognitive or behavioral etc. functioning or state/s is attained etc.), including any smart electrode or open or closed loop improvement/s or normalization/s of optimization/s or brain or associated functioning or threshold/s or state or disease/s or health or enhancement configuration allowing for the control or improvement/s of any targeted or diseased or abnormal or impaired brain or cognitive or behavioral or other brain functioning. This configuration of the invention also allows for the deciphering or diagnosis or interpretation or decoding or deciphering any brain or neural or neuronal or neural-network operation of activity or any single or multiple or any spatial-temporal or spatial or temporal activity or process of behavior or cognition or motor or any other neurophysiologic or electrophysiological brain functioning or disease/s or diseased state or feature or abnormal or supernormal or enhanced brain of associated cognitive or affective or motor or behavioral or intellectual or mental health or personality or mood or balance or any other activity or pattern/s or state/s or condition/s or performance/s or level/s etc.

Finally, it is important to note that the current invention also allows for a series of possible applications and/or claims which include but are not limited to:

a) The Specificity Principle:

which is capable of identifying or deciphering and/or manipulating and/or altering and/or healing etc. any specific single or multiple spatial- and/or temporal and/or spatial-temporal electrophysiological or neurophysiologic and/or any single or multiple and/or single or multiple spatial-temporal activation or association of pattern/s associated with any single or multiple cognitive or affective or motor or intellectual or any other brain- or brain-related activity or feature/s or pattern/s or function/s or disease/s or healthy or abnormal or subnormal or above normal or normal brain functioning/s or feature/s and/or which is capable of identifying or deciphering or interpreting any such single or multiple spatial- or temporal- or spatial-temporal and/or electrophysiological or neurophysiologic or neuronal or neuron/s or neural network/s or any brain activity/ies or brain function/s and/or any single or multiple cognitive or behavioral or motor or affective or neurophysiologic meaning/s or function/s or activity/ies or input/s or output/s or process/es or processing/s or pattern/s and/or which is capable of utilizing this specific single or multiple spatial-temporal brain activity/ies and/or any electrophysiological pattern/s or activity/ies and/or any associated single or multiple cognitive or affective or neuronal or neuron/s or neural-network/s or any other brain function/s or activity/ies—for the purpose/s of modulating and/or improving and/or enhancing any specific single or multiple such spatial-temporal and/or electrophysiological and/or same- or different-brain or neural or neuronal or electrophysiological or neurophysiologic etc. or cognitive or affective or motor or intellectual or any other targeted brain function/s or feature/s or pattern/s and/or including in real-time or off-line and/or continuously or intermittently or otherwise monitoring and/or modulating and/or altering including through feedback or control or adjustment or fine-tuning or optimizing or normalizing feedback loop/s or control mechanism/s capable of modulating any such single or multiple specific spatial-temporal and/or electrophysiological and/or neurophysiologic and/or any cognitive or affective or behavioral or motor or brain related process/es or function/s or any input/s and/or output/s or mechanism/s or pattern/s or disease/s or disease-related or health or healthy or targeted norm/s or function/s or pattern/s or feature/s or any desired brain function/s or interpretation of interface/s or capacity/ies.
1) Personalized Brain Medicine: A system or method or medical device or apparatus/es or electrode/s or smart electrode/s or mechanism/s or configuration/s which may consist of any single or multiple or any combination of claim/s #1 through #20 which is capable of personalizing any single or multiple and/or spatial- and/or temporal- and/or spatial-temporal deciphering/s and/or interpretation/s and/or stimulation/s (including but not limited to any combination of electrical or electromagnetic or neuronal or cognitive or affective or behavioral or motor or neurophysiologic or other brain stimulation/s) and/or manipulation/s and/or alteration/s and/or healing/s and/or modification/s and/or attainment/s- or improvement/s- or normalization/s- and/or optimization/s- and/or enhancement/s-towards any desired brain or single or multiple and/or any spatial-temporal pattern/s or configuration/s or neuronal or neural or neuron/s norm/s (e.g., including but not limited to health or healthy or normal or normalized or optimized norms) pertaining to any single or multiple and/or any spatial-temporal and/or any electrophysiological or neurophysiologic and/or neuronal and/or cognitive or behavioral or affective or motor or any intellectual or any other brain and/or neural and/or neuronal and/or brain-related and/or directed towards attaining or improving any such single or multiple spatial-temporal and/or any associated any single or multiple electrophysiological or neurophysiologic or cognitive or affective or motor etc. any healthy- or diseased- or any improvement or perfection or optimization of normal or subnormal or above-normal brain and/or neuronal and/or neuron/s and/or cognitive or affective or behavioral or motor or any other brain functioning; The claim for personalized brain medicine also includes and/or implies and/or consist/s of the capacity to identify or decipher or interpret any single or multiple and/or spatial-temporal and/or any neuron/s and/or neuronal and/or neural-network/s and/or any single or multiple locus or loci or any cellular or neuronal activity/ies or pattern/s or electrophysiological or neurological or neurophysiologic pattern/s or parameter/s (including but not limited to any structural o functional or connectivity or neurophysiologic or behavioral or cognitive or affective or motor or other neuronal or neuron/s or any brain or brain related or associated activity/ies)—including in real time or continuously or off-line or intermittently or at any targeted or identified single or multiple spatial- or temporal or spatial-temporal point/s or interval/s or duration/s etc. and/or which is capable of modulating or affecting change/s or producing therapeutic or positive or improving neuroplasticity or improving any functional or structural or electrophysiological neural or neuronal or neuron/s or any brain (localized or non localized or generalized or particular) function/s or functioning or improving any impaired brain or cognitive or behavioral or neuronal or neurophysiologic or electrophysiological or any diseased or impaired or abnormal or subnormal brain functioning or feature/s or characteristic/s or cognitive behavioral etc. performance/s or state/s or condition/s or enhancing any brain or cognitive or motor or behavioral or affective or other brain function/s of functioning and/or including through the personalization of any single or multiple neuronal or brain or cognitive, affective, behavioral etc. measurement/s or computation/s or deciphering of neural or brain or cognitive affective etc. activity/ies or pattern/s or interpretation/s etc. and/or modulation/s or manipulation/s or treatment/s or therapeutic intervention/s or stimulation/s (including all or some of the above mentioned or application mentioned electrical or electrophysiological or neurophysiologic or electromagnetic or cognitive or behavioral or affective etc.) and/or in order to improve personalized or generalized or clinical group characteristic/s to which the personalized computation/s or identification of deciphering/s indicate belongingness or compatibility etc. towards a set or adjustable target/s or goal/s or aim/s or level/s or parameter/s (including but not limited to attaining an improved electrophysiological or neurophysiologic or behavioral or cognitive or motor or affective or any other single or multiple brain localization or pattern/s and/or any single or multiple spatial-temporal pattern/s or loci or locus etc. or towards healing diseased or impaired neural or brain or cognitive etc. function/s or functioning etc.) and/or including a personalized feedback or control loop/s and/or stimulation parameter'/s' norms based stimulation or modulation or control mechanism/s or feature/s etc.
2) Cortical-Sub cortical Controlling Brain Disease/s or Enhancement/s Treatment: A system or method or medical device or electrode/s or smart electrode/s or mechanism or apparatus/es or configuration including but not limited to any single or multiple claims #1 through #21 (or any combination/s of these claim/s separately or together—with or without any other element/s) which is capable of stimulating or enhancing or improving or attaining any targeted brain and/or neuron/s or neuronal or cognitive or affective or behavioral or motor or any other brain feature/s or function/s or state/s or condition/s including improving or healing or optimizing or normalizing any diseased or subnormal or abnormal or impaired brain function/s or feature/s or state/s or condition/s or characteristic/s and/or attaining or improving towards or modulating any brain function/s or neural state/s or improving any brain disease/s and/or including through or based on or including the identification and/or computation and/or determination of any single or multiple diseased or disease-related or impaired or abnormal or to be enhanced brain region/s or pattern/s or feature/s and/or the possible modulation or therapy or treatment or healing of any such single or multiple diseased brain region/s or neuron/s or neuronal connection/s or neural network/s and/or any associated or converging or same- or different-single or multiple cognitive or affective or behavioral or motor or intellectual or any other impaired or diseased or abnormal or subnormal or normal or to be enhanced brain function/s including through the stimulation of same- or different-brain regions and/or modulation of diseased brain function/s or pattern/s or features and/or stimulation or modulation (including electromagnetic or electrical or neuronal or cognitive or affective or behavioral or motor or any other brain stimulation/s) of any single or multiple brain region/s or locus or loci or neuron/s or neural network/s etc. including but not limited to: cortical brain region/s or locus or loci or controlling neuron/s or neural-network/s which control or affect or modulate or heal or treat or regulate or normalize any brain disease/s abnormality/ies or specific impairment/s or deficiency/ies or diseased state/s or condition/s; This may include also a real-time or off-line or continuous or feedback or control loop/s based control or modulation/s or stimulation of any cortical or other neural or neuron/s or brain or neural-network/s multiple spatial- or temporal- or spatial-temporal brain region/s or neuron/s or neuronal or associated cognitive or behavioral or affective or motor or any other brain activity/ies or pattern/s which is adjusted or computed or stimulated or determined or executed or fine-tuned personally for each individual who is diagnosed or evaluated or computed to deciphered to have any specific single or multiple spatial- or temporal- or spatial-temporal impaired or diseased or abnormal or dysfunctional or to be enhanced brain or neuron/s or neural-network/s including until a certain or particular improvement/s or targeted or enhancement/s or modulation/s of diseased or abnormal or impaired or to be enhanced brain function/s or neuronal or neuron/s or cognitive or behavioral or motor or neurophysiologic function/s or pattern/s have been achieved etc.; a few examples for this Personalized Brain Medicine could include Personalized smart electrode/s or invasive or non-invasive for Parkinson's disease or autism or schizophrenia, or ADHD or depression, or PTSD.
3) Critical CNS Pathway/s or Junction/s or Region/s Control or Optimization: A system or method or medical device/s or apparatus/es or configuration/s including but not limited to any single or multiple of claim/s #1 through #22 which is capable of identifying or deciphering or computing any single or multiple critical pathway/s or region/s or neuron/s or neural-network/s or junction/s or critical single or multiple electrophysiological spatial-temporal diseased or impaired brain region/s and/or identifying any single or multiple and/or spatial- or temporal- or spatial-temporal-diseased or impaired or abnormal or subnormal brain region/s or neuron/s or electrophysiological or neurophysiologic or cognitive or affective or motor or behavioral etc. pattern/s or normal condition/s or to be enhanced and/or identification/s or deciphering/s or computation/s of any single or multiple and/or spatial-temporal combination/s or pattern/s or loci or locus or neuron/s or neuron's/s' activity/ies which may be associated with any particular brain disease/s or brain impairment/s or associated neuron/s or neurological or cognitive or behavioral or affective or motor or other abnormality/ies or deficiencies or any feature/s to be enhanced, and which is also capable of stimulating the same- or different-single or multiple and/or spatial- or temporal- or spatial-temporal critical pathway/s or junction/s or region/s or point/s or any electrophysiological or neurophysiologic or cognitive or affective or behavioral or motor or any other brain- or neural-activity/ies or pattern/s which are associated with the critical pathway/s or region/s or juncture/s or specific disease/s or impairment/s or brain condition/s or pathological or abnormal brain function/s or feature/s or characteristic/s, and/or which are capable of modulating or altering or positively affecting or healing or treating or improving any brain disease/s or brain disease/s specifically associated or causing or linked critical disease/s or diseased brain region/s or point/s or juncture/s or pathway/s or neuron/s etc. or which is capable of attaining a certain criterion or criteria for improved or targeted or healthy or enhanced brain function/s or disease-ridden or (fully or partially) eliminated disease/s or diseased brain condition/s or pathway/s or juncture/s or any associated single or multiple spatial-temporal electrophysiological or neurophysiologic or cognitive or behavioral or motor or any other brain related function/s or feature/s or state/s or condition/s or pattern/s; Further application of this principle claim is the identification/s or deciphering/s of any CNS controlled or modulated or stimulated disease/s in the brain or body or mentally or Psychiatric (or for enhancement/s of any normal or below normal or abnormal or subnormal or above-normal cognitive or behavioral or bodily or health-related or any function/s or feature/s of the organism or of any of its spatial- or temporal- or spatial-temporal function/s or activity/ies or state/s or condition/s or feature/s or characteristic/s)—which may also include the identification of the precise specific single or multiple spatial- or temporal- or spatial-temporal CNS combination/s or electrophysiological or cognitive or affective or behavioral or motor or health-related function/s or disease/s or illnesses or impairment/s or abnormality/ies or pattern/or to be enhanced function/s or features pattern/s or characteristic/s, and which may also include the modulation or stimulation (including but not limited to any single or multiple electrical or electromagnetic or cognitive or affective or behavioral or motor or any other brain stimulation/s) of such same- or different-single or multiple spatial- or temporal- or spatial-temporal neural or CNS or any single or multiple spatial- or temporal- or spatial-temporal activity/ies or pattern/s or disease/s or diseased state/s or condition/s or function/s, including also the capacity to personalize or to produce control loops or control mechanism/s for controlling or eliminating any disease/s or disease-state/s or condition/s or to optimize or normalize or improve any such single or multiple brain disease/s or condition/s or any CNS associated or controlled or modulated or affected or healed body related disease/s or impairment/s or abnormality/ies etc. (including but not limited to a continuous or intermittent or fixed or otherwise controlled feedback or control loops capable also of producing total or partial healing or remission or improvement/s in any such single or multiple brain or CNS or CNS related or bodily disease/s controlled or affected by the CNS function/s or pattern/s or locus or loci etc.). A few examples for the scope of this invention or possible application/s include the identification and/or deciphering of any CNS or brain or region/s and/or bodily disease/s or impairment/s or disease/s or abnormality/ies such as any type of cancer/s or cardiovascular or infectious disease/s or hormonal or stress, mood, cognitive or affective or motor or personality or mental state or psychiatric related or associated bodily and/or CNS disease/s for which there is an identification/s or ciphering/s or deciphering or computation/s of the specific single or multiple spatial- or temporal- or spatial-temporal diseased or disease/s or impaired or abnormal or deficient or pathological or to be enhanced CNS or neural or neuron/s or pattern/s or function/s and which may also include the possibility of stimulating any such same- or different-single or multiple spatial- or temporal- or spatial-temporal diseased (or to be enhanced) brain or neural or neuron/s or any CNS point/s or region/s or any CNS or brain or cognitive or affective or motor or any CNS function/s or pattern/s or spatial-temporal configuration/s or state/s or condition/s in order to optimize or enhance or improve or heal or effectively treat any brain and/or CNS and/or bodily and/or physical and/or mental and/or psychiatric and/or well-being and/or intelligence and/or condition/s (including through control loop/s or feedback loop/s as described above).

A system, method, medical device or apparatus or any other mechanism or mechanisms capable of stimulating selectively specific diseased or healthy or targeted brain region or regions for the purpose of normalizing or optimizing of affecting positively or therapeutically any single or multiple structural, functional or behavioral or electrophysiological deficiency or disease or impairment or functional or structural or behavioral or other abnormality or dysfunction or disease or diseases or illness or illnesses; This method or system or medical device etc. is capable of improving or healing or affecting (positive) neuroplasticity or assisting in cognitive or behavioral or functional or structural normalization or countering or resisting or treating any brain related (or bodily) disease can operate through the selective stimulation of particular disease or diseased or affected or healthy brain regions (such as the Substantia Nigra in the case of Parkinson's disease) in order to optimize the functional activation level of the diseased brain region or in order to improve or heal or optimize the behavioral or movement or affective or cognitive or bodily or electrophysiological index or level or indices or levels or feature or features or abnormality or abnormalities etc.; Such therapeutic or improvement-inducing or optimization or disease- or diseases- or abnormality- or abnormalities-counteracting or resisting or improving or enhancement of diseased or deficient brain or behavioral or cognitive or affective or neurophysiologic function or functions may be achieved or affected or modulated through the synchronization or modulation or fine-tuning or optimization of electrical or electromagnetic stimulation in the affected or normal diseased brain region or regions based on a pre-set criterion or criteria, or interactively fine-tuned or modulated based on response to certain electrical or electromagnetic or behavioral or cognitive or neurophysiologic or electrophysiological stimulus or stimuli or set on ‘real-time’ or ‘off-line’ to reach or improve towards or approach or enhance or normalize any level or levels or criteria or criterion of electrophysiological or behavioral or cognitive or physiological or movement or bodily or functional or structural or other index or indices etc.; The measurement and/or the fine-tuning of (set or interactive or adjustable or fixed or intermittent or fixed or other) level/s or feature/s or intensity or intensities or pattern or pattern/s of activity or functional or electrophysiological or cognitive or behavioral or physiological or motor or bodily or function or structure etc. in the determined diseased brain region or regions or in any associated or other healthy region or regions can be performed simultaneously or before- or after- the electrical or electromagnetic stimulation of the determined or other brain region or regions and can interact or affect or modulate or not affect, interact etc. with the ongoing, or dynamic or fixed or intermittent stimulation or single or multiple diseased or healthy region/s or may be performed ‘off-line’ at different time/s then the actual stimulation time-point/s or interval/s or regimen, and can be performed at single or multiple or same- or different-spatial and/or temporal-point/s or loci or locus relative to where electrical or electromagnetic (or cognitive or behavioral or movement or other bodily or physiological stimulation) takes place. The determination of the uniform or personalized or disease- or location- or target-specific etc. properties of electrical or electromagnetic or cognitive or behavioral or physiological etc. stimulation can be determined based on real-time or off-line or known in the art or investigated or experimented norms or curves or intensity/duration/location etc. that are necessary in order to reach a particular targeted pattern or level or levels or feature/s of electrophysiological or cognitive or behavioral or physiological or movement or other criteria or criterion or improvement or optimization towards such criteria, criterion etc.

Insulated biogenic smart systems and personalized biogenic ‘markering’ system comprising a system, method, or medical device or pharmaceutical/s or drug delivery system which is based on an insulation of identified pathogenic or diseased or abnormal or functionally or structurally impaired bodily or organ/s or tissue/s or brain or neuronal loci or locus or region/s or organizational or functional structure/s or network/s; Such system is capable of insulating the diseased or pathogenic or impaired or abnormal or dysfunctional locus, loci, region/s, cell/s or structure/s through various (known or to be developed) methodologies, apparatus/es, membrane/s, isolating chemicals, device/s, implant/s, electrical or electromagnetic or laser or sound or other neurophysiological, immunological, chemical, or endocrine-based insulation methodology/ies or technique/s; Such system may also have the capacity to saturate or deliver in real-time different levels or dosages or adjustable value/s or level/s of any particular pharmaceutical/s or other chemical or biological element/s or genetic or microbiological or other element/s or substances which may be specific to a particular site or region/s or cell/s or stage of a disease or personalized to the specific pathogenesis or biological abnormality or functional deficiency or abnormality or biological state or specific abnormality or pathological measures or value/s measured at a particular region/s or cell/s or organ or functional organization or systemic level/s; Finally, the delivery of the precise dosage/s or level/s or compound/s to the insulated biological or biogenic pathological or diseased region/s or regions may be accomplished either through an ‘in-store’ reservoir or storage of pocket/s or capsule/s or any other implanted, in-vitro pool of the specific compound or compound/s, or may be derived or delivered or transported or infused through intermittent or infrequent or constant invasive or non-invasive delivery of the substance or may be intermittently, or periodically of continuously delivered through various infusion, osmosis, blood, cellular, immunological, genetic, viral or other means for transporting or magnetizing or drawing any specific chemical or other bodily, biological, genetic or other compound to the desired pathological site/s or region/s or organ/s etc.; Such delivery may also be controlled via internally or automatically or externally or manually or physician-aided single- or multiple—time measurements of the levels of a particular element or compound/s or biological state etc. and may be adjusted accordingly (e.g., in real-time or ‘off-line’). Thus, the described INSULATED BIOGENIC PHARMACEUTICAL SMART SYSTEM possesses capabilities to insulate identified pathological biological site/s, organ/s, tissue/s cell/s biological network/s in a manner which may be tailored to an individual or a disease/s or a stage/s in the progression of a disease/s and may also be adjusted to allow for specific (constant or adjustable or intermittent) level/s, dosage/s, or element/s or compound/s or genetic or biological or neurophysiological of chemical or microbiological or any other single or multiple or distinct or combined chemical or physical element/s which therefore may be made precisely compatible or congruent with the healing or improvement or therapy or pharmaceutical treatment of any particular disease/s or region/s or cell/s or individual/s or loci or locus of any abnormal disease/s region/s, cell/s, tissue/s, organ/s etc.

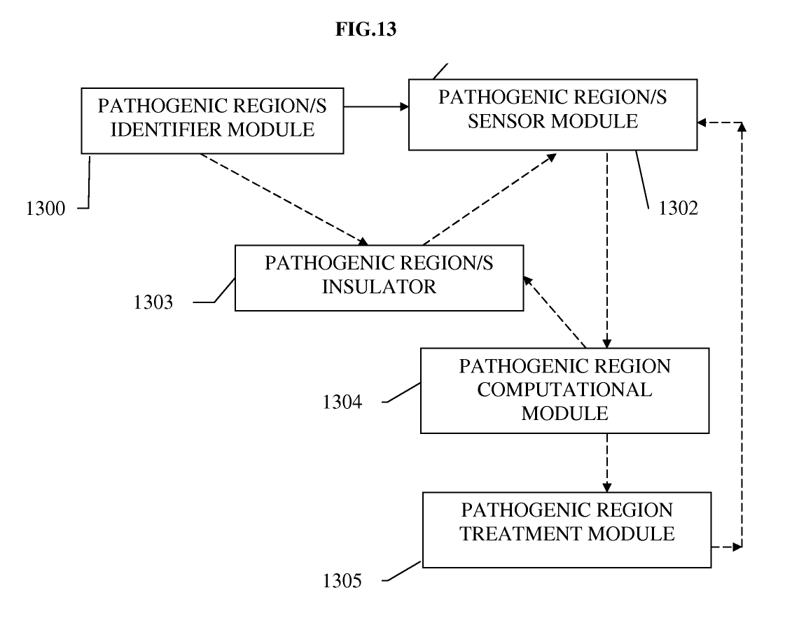

Reference is now made to FIG. 13 which describes a PATHOGENIC REGION/S IDENTIFIER MODULE 1300 which is capable of identifying single or multiple bodily region/s or organ/s or cell/s or cellular organization/s or functional organization unit/s within the body or any other single or multiple locus or loci within the human body, or any of its systems or the brain or nervous system or any other system or organ, tissue or cell/s within the body that are impaired, diseased, pathological, abnormal, deficient, malfunctioning, abnormal in structure or function, or connectivity or any other biological aspect/s function or trait in an individual or in a group of patients or in a particular age/s or stage/s of any single or multiple disease/s in a single individual or group/s of individual/s. The PATHOGENIC REGION/S IDENTIFIER MODULE 1300 may identify the diseased bodily region/s, locus, loci, cell/s, network or organ/s or segment/s of any of the abovementioned or of the body etc. based on a comparison of that particular region/s, organ/s, tissue/s or any other single or multiple, disjoint or independent or connected bodily part/s, loci or locus by comparing its function, structure, chemical, physical, electrical or electromagnetic or light-related or sound-related or neurophysiological or imaging based to that of normal health criteria (e.g., for each of these aspect/s or dimension/s), or based on a physician or clinical or computerized or radiological, histological, chemical, genetic, hormonal, neurotransmitter/s biometric, blood or any other medical test or examination etc. or any other medical or clinical test or measurement or analysis (which is capable of determining pathological or abnormal functioning or disease related to any particular region/s or cell/s or organ/s etc.). Following the identification of the pathogenic, pathological or abnormal or impaired or diseased single or multiple bodily region/s in an individual the PATHOGENIC REGION/S IDENTIFIER MODULE 1300 directs the PATHOGENIC REGION/S SENSOR MODULE 1302 the precise mean/s or method/s or region/s or loci or locus or value/s or measurement/s or test/s or medical device/s or methodology to measure the abnormal or impaired or diseased or pathological site/s, value/s, substance/s or compound/s or element/s or analysis/es of a single or multiple bodily region/s, locus, loci, function/s or structure/s or cell/s or system/s or organ/s etc. in order to obtain real-time or off-line data or measurement/s of any single or multiple disease/s or ailment/s or abnormality/ies or deficiency etc.; The PATHOGENIC REGION/S IDENTIFIER MODULE 1300 may also direct the PATHOGENIC REGION INSULATOR 1303 the precise mean/s or method/s or site/s or single or multiple region/s or method/s or technique/s or application/s which are aimed at insulating or blocking off or isolating or barring or insulation (e.g., partial, full, intermittent, constant or adjustable or dynamic or fixed or other) of a given particular region/s or organ/s or cell/s or tissue/s or any part/s or segment/s of any of these biological or bodily locus or loci or single or multiple region/s, cell/s, structure/s, system/s, sub-system/s etc. which may be accomplished via different chemical, membrane or other sealing or isolating or insulating or disrupting the passage of any chemical or other compound etc. The PATHOGENIC REGION/S SENSOR MODULE 1302 may be installed or implanted or attached (e.g., invasively or non-invasively) to the relevant or appropriate or specific single or multiple region/s or loci or locus before-during-after or in connection or independently of the PATHOGENIC REGION INSULATOR 1303 or the PATHOGENIC REGION COMPUTATIONAL MODULE 1304. This is due to the fact that the PATHOGENIC REGION/S SENSOR MODULE 1302 measurement/s or indication/s or value/s or test/s or extraction of any biological or chemical or physical substance/s or element/s or compound/s may impact or determine or interact or influence the PATHOGENIC REGION COMPUTATIONAL MODULE 1304 determination of the dosage/s, or value/s or frequency/ies or compound/s or chemical/s or pharmaceutical agent/s to be released or delivered or targeted towards a specific region/s or loci or locus or organ/s or cell/s or tissue/s or any segment/s thereof—which may therefore inform or direct or control or modulate the placement/s or operation/s or modulation/s or location/s or frequency/ies, dosage/s or other delivery or compound-specific or location/s or stage/s or disease/s specific administration of the drug/s or compound/s which may closely interact with the placement or operation of interaction of the PATHOGENIC REGION INSULATOR 1303 independently or interactively or jointly or dis-jointly with the PATHOGENIC REGION TREATMENT MODULE 1305.

The PATHOGENIC REGION SENSOR MODULE 1302 is capable of measuring any physical, chemical, histological, hormonal, genetic, biological, microbiological, viral or inflammatory or any other chemical or neurophysiological or electrical or electromagnetic measurement of the identified diseased or impaired or abnormal or deficient or pathological bodily or brain or single or multiple region/s or cell/s or tissue/s or loci or locus within the human body—at single or multiple time point/s or off-line or in real-time or in response to any sort of stimulation or state or condition or independently etc. These measurement/s by the PATHOGENIC REGION SENSOR MODULE 1302 are then transferred to the PATHOGENIC REGION COMPUTATIONAL MODULE 1304 which is capable of determining whether or not the particularly measured chemical, biological, histological, or any other physical substance or electromagnetic or electrical values measured for the single or multiple diseased bodily or brain or any other organ/s or tissue/s or cell/s or loci or locus are normal or abnormal and what may be the particular abnormality/ies, deficiency/ies, or impairment/s or disease/s or imbalance/s characterizing this organ/s or region/s or loci or locus or system/s or sub-system, tissue/s, cell/s etc. Based on the results of this analysis by the PATHOGENIC REGION COMPUTATIONAL MODULE 1304 the PATHOGENIC REGION COMPUTATIONAL MODULE 1304 outputs to the PATHOGENIC REGION TREATMENT MODULE 1305 the precise abnormality or abnormal or deficient or impaired chemical, substance, neurophysiological, hormonal, blood-related or any other fluid related, genetic, cellular, histological, or other structural or functional or disease related or age related or other physiological or biological abnormality or pathology that is associated or detected within the specific Pathogenic regions at any time or times (which may be obtained through ‘off-line’ or ‘real-time’ measurement/s of the PATHOGENIC REGION/S SENSOR MODULE 1302).

Based on the output of the PATHOGENIC REGION COMPUTATIONAL MODULE 1304 which can identify the specific pathogenic or pathological or abnormal or impaired bodily or brain or any other bodily system, sub-system, region/s or cell/s or cellular or microbiological or genetic abnormality/ies or deficiency/ies or any other chemical, physical, structural or functional or histological or any other substance etc. the PATHOGENIC REGION COMPUTATIONAL MODULE 1304 can determine the location, type, operation, function, partial or full or intermittent or continuous or dynamic changes in the permeability or insulation or isolation or blockage or containment of specific or all or single, few or multiple substance/s or chemical/s or passage of fluids or liquid/s or gas/es or blood or any other bodily substance or pharmaceutical or drug or electrical or electromagnetic or any other macroscopic or microscopic or any size or molecule or compound or interaction or flow or transmission of element/s or natural or artificial material/s surrounding or insulating or isolating or sealing off (e.g. partially or fully or permeable) a specific pathological or diseased or disease-related single or multiple bodily or body region/s or network/s or illness; Hence, based on the PATHOGENIC REGION COMPUTATIONAL MODULE 1304 analysis and output of the disease/s or diseased region/s or pathology/ies or abnormality/ies the PATHOGENIC REGION COMPUTATIONAL MODULE 1304 directs- or outputs to- or controls- the PATHOGENIC REGION/S INSULATOR 1303 which can be any sheath or membrane or any element or any structure (e.g., microscopic, macroscopic, permeable or totally insulating etc.) which is capable of filtering or blocking off or damning or insulating or isolating or producing any sort of permeability or filtering or osmosis or letting in- or out- or containing of blocking (partially or fully or dynamically changing) a single or multiple pathogenic or diseased or disease-related or abnormal or identified or pathological it impaired region/s by blocking passage of all substance/s or some or a single substance or any other physical or bodily or pharmaceutical or electrical/electromagnetic or thermal or auditory, tactual, sensory, olfactory, pain related or other substance or any other physical effect or chemical compound or element from the insulated or filtered or blocked or sealed pathogenic region/s to its surrounding or adjacent or even distant or distantly connected bodily region/s. The control of the site/s or locus or loci or function or passage of any physical or chemical or electrophysiological or any other bodily or natural or artificial or chemical compound or element or substance or genetic or cellular or blood related or any other microscopic or macroscopic element/s from the insulated pathological or diseased or abnormal or infected or identified body or bodily region/s loci or locus to its surrounding environment or vice versa from the environment to the insulated region/s is carried out by the PATHOGENIC REGION COMPUTATIONAL MODULE 1304 which is capable of determining which precise region/s or site/s or loci or locus or specific chemical element/s or compound/s or physical transmission of heat, electrical or electromagnetic or histological or microbiological or genetic or protein or other element/s, compound/s or physical effect between the insulated or identified or diseased pathogenic or pathological bodily region/s and its environment (e.g., in either or both direction/s). One example for this insulation of the pathogenic region/s and its environment/s or extra- or intra-pathogenic region/s space/s or cell/s or network/s is the insulation or blockage (partial or full or dynamically changing or stable) of the pancreas or the heart (or region/s of these organs) or of the hippocampus or pituitary gland etc. Such partial or full, permeable or filtering—of a single or a few or multiple element/s or materials or compounds or molecule/s may be predetermined by the PATHOGENIC REGION COMPUTATIONAL MODULE 1304 (including as a set, changeable, constant or variable or adjustable etc. level/s or threshold/s or certain single or multiple or varied compound/s or chemical/s) or may be adjusted according to adjustable or dynamic or varied or interactive computations of the PATHOGENIC REGION COMPUTATIONAL MODULE 1304 derived the measurement/s of the PATHOGENIC REGION/S SENSOR MODULE 1302 measurement/s of specific pathogenic or diseased bodily region/s such as the pancreas, hippocampus, heart or sub-region/s or subsystems or specific cell/s, region/s within these organ/s or tissue/s or network/s etc. Thus, in the case of for example the pancreas the insulation of the pancreas or subregion/s of the pancreas by the PATHOGENIC REGION/S INSULATOR 1303 may allow for the blockage or filtering or partial or full filtering or disabling of particular substance such as the insulin to be released from- or to- the pancreas to- and from- its biological environment or blood stream or vessel/s or any cell/s within- or outside- the pancreas. Such insulation or filtering or isolation of the pancreas (in this example) from its environment or biological interaction or output or input of specific factors (such as insulin generating factor/s) or determination or allowance of only below or above a certain threshold of level/s of a specific substance/s or of filtering of molecule/s magnitude exceeding or below a certain size/s etc. may allow for a build up or filtering of therapeutic or pharmaceutical drug delivery or saturation of exposure to specific therapeutic element/s or avoidance or elimination of toxic or antigen-based on infectious or contagious or harmful element/s.

The functioning of the PATHOGENIC REGION/S INSULATOR 1303 may be combined together with the adjustable or constant or fixed or intermittent or varied functioning of the PATHOGENIC REGION TREATMENT MODULE 1305 which is directed by the PATHOGENIC REGION COMPUTATIONAL MODULE 1304 and which is capable of increasing or enhancing or selecting an increase or decrease or maintenance of a specific single or multiple compound/s or element/s or material/s or substance/s within or without or inside, or outside or associated with the PATHOGENIC REGION/S INSULATOR 1303 of specifically selected pathogenic region/s or diseased region/s or disease-related region/s which may be accomplished through a combination of the PATHOGENIC REGION COMPUTATIONAL MODULE 1305's control of the degree of permeability or selection of specific substance/s or element/s or filtering of any molecule/s, element/s, genetic or biological or bodily substance or pharmaceutical/s drug/s or infusion/s or elimination/s of any particular substance or chemical/s etc. or of the particular level/s of a single or multiple or mixed combination or specification of a specific level or threshold/s of a mix of a series of compound/s, element/s or molecule/s etc. which may be fixed or changed or dynamically determined etc.—in combination with the PATHOGENIC REGION TREATMENT MODULE 1305 which can adjust or coordinate or interact or be associated with or enhance or interfere with the selection or maintenance or enhancement or reduction or change in the composition/s or level/s of a particular substance/s or element/s or chemical/s or pharmaceutical/s or toxin/s, infectious related substance/s or carcinogen element/s or viral or genetic or microbiological substance/s or molecule/s etc. (including as described above) such that the coordination or the filtering or permeability or blockage or insulation or containment of particular element/s or compound/s or molecule/s by the PATHOGENIC REGION/S INSULATOR 1303 together or interacting or in addition to the PATHOGENIC REGION TREATMENT MODULE 1305 selection of infusion or addition or increase in the level/s of a single or multiple compound/s or element/s or molecule/s or substance/s or genetic component/s or any other physical or chemical element/s or elimination of extraction of decrease or suction or decrease in the level/s or composition or selection of specific element/s or chemical/s as coordinated or controlled or determined by the PATHOGENIC REGION COMPUTATIONAL MODULE 1304 is capable of determining the particular composition/s or level/s or increase/s or decrease/s in a single or multiple element/s or compound/s or creation of a specific biological or ph level or cellular or intra- or extra- or cellular or organ/s intra- or extra-biological, fluidic, or metabolic or genetic or nutrient or certain chemical or organic environment or composition etc. which may also be conducive to therapeutic application/s or healing or improvement in biological or organic or cellular of bodily or body related functioning etc.

This configuration of identified or selected pathogenic region/s or diseased region/s or disease related region/s in the body or loci or locus of disease or diseased region/s that are insulated by the PATHOGENIC REGION/S INSULATOR 1303 as determined by the PATHOGENIC REGION/S COMPUTATIONAL MODULE 104 and as directed before- or during- or after- the PATHOLOGICAL REGION/S SENSOR MODULE 1302 and which is further controlled or mediated by the PATHOGENIC REGION/S COMPUTATIONAL MODULE 1304 control or direction of the PATHOGENIC REGION TREATMENT MODULE 1305 selection of particular chemical/s or other substance/s or molecule/s or level/s of any single or multiple or series of imprint or collection of substance/s or element/s or compound/s within the PATHOGENIC REGION/S INSULATOR 103 or outside of it or associated with it in some way and which may be further controlled or adjusted through a fixed or predetermined or adjusted in time or interacting or varying based on single time/s or spatial measurement/s at a single or multiple locus or loci etc. within or without the PATHOGENIC REGION/S INSULATOR 1303 measurement of the level/s of particular element/s or substance/s or molecule/s—may allow to create a well controlled biological, or cellular or intra- or extra-cellular or blood, lymphatic, or any other body or bodily environment/s within particular region/s of the body as desired or determined by the system or a physician or a physician aided system etc. This configuration may be able to produce an ‘in-vitro’ like procedure or environment or manipulation within the human (or indeed any other organism) allowing the controlling of biological or chemical or genetic etc. or any body- or bodily-functioning environment which is within the living body yet allows for an in-vitro like control of the precise level/s or composition/s of particular single, series or multiple biological, natural or artificial, pharmaceutical, disease-related, disease producing (including viral or cancer or other disease associated element/s or factor/s or producer/s or by-products etc.) or disease/s antagonistic or combating or therapeutic or pharmaceutical or immunological or antigen-fighting or counteracting element/s etc.

As may be seen from FIG. 13 the system described can operate in an interactive manner such that the selection of the PATHOGENIC REGION/S INSULATOR 1303 may be selected or modulated or formulated or controlled (e.g., including but not limited to the location/s, or type of filtering or permeability or electrical or electromagnetic filtering or magnetic attraction or repulsion or laser or ultrasound associated methodologies for filtering any single or multiple of series of environment determined criteria for determination of which element/s or molecule/s etc. or level of substance/s may be allowed to pass through the PATHOGENIC REGION/S INSULATOR 1303 into- or out of-within-or without- the PATHOGENIC REGION/S INSULATOR 1303—which may be controlled through either the PATHOGENIC REGION/S IDENTIFIER MODULE 1300 and/or the PATHOGENIC REGION COMPUTATIONAL MODULE 1304 (e.g., the latter of which may be interactively modulated or controlled or adjusted through the operation or input from the PATHOGENIC REGION/S SENSOR MODULE 1302 which may measure the level/s or composition/s of particular level/s or existence of specific element/s or compound/s or chemical/s within-without-etc. the PATHOGENIC REGION/S INSULATOR 1303 at different time point/s, interval/s and/or location/s loci or locus or region/s which may be diseased or abnormal or adjacent or communicating or associated with such identified diseased region/s as identified by the PATHOGENIC REGION/S IDENTIFIER MODULE 1300); The PATHOGENIC REGION COMPUTATIONAL MODULE 1305 also coordinates or controls or monitors or interacts with the PATHOGENIC REGION TREATMENT MODULE 1305 in order to produce the desired (predetermined, interactively or in real-time determined or intermittently determined or constant or variable or adjustable or dynamic etc.) biological or pathogenic region/s chemical or genetic or pharmaceutical or antigen- or immunological- or histological- or microbiological- or physical environment or composition or compound/s or in-vivo in-vitro like environment or chemical or biological composition etc.

As may be further seen from FIG. 13 the interaction or control or modulation input of the PATHOGENIC REGION/S SENSOR MODULE 1302 may be adjusted intermittently or continuously or interactively by the PATHOGENIC REGION TREATMENT MODULE 1305 which may in turn control or modulate or interact with- or adjust the PATHOGENIC REGION COMPUTATIONAL MODULE 1304 which may in turn further interact with- modulate—or control- the PATHOGENIC REGION/S INSULATOR 1303 and/or the PATHOGENIC REGION TREATMENT MODULE 1305 which may further adjust-interact-control-output-to the PATHOGENIC REGION/S SENSOR MODULE 1302 with additional feedback loop or adjustment capabilities from the PATHOGENIC REGION/S INSULATOR 103 which receives input from the PATHOGENIC REGION COMPUTATIONAL MODULE 1304 and outputs or adjusts the PATHOGENIC REGION/S SENSOR MODULE 1302.

The system's configuration allows to predetermine or adjust or measure and interactively change or alter or modulate the composition/s, value/s or assembly or series of element/s, chemical/s, biological element/s, compound/s, molecule/s or any other natural, disease-associated or antagonistic- or therapeutic or medical intervention or increase or decrease in any element/s or substance/s or compound/s or molecule/s that is to be measured or found within- or without-associated with- or interacting with or produced or maintained or eliminated or communicated or instigating any compound/s or element/s or substance within or associated with or outside of the pathogenic region/s or diseased region/s or organ/s or sub/system/s etc. or within-without or associated with the region/s or loci or locus of the PATHOGENIC REGION/S INSULATOR 1303, thereby allowing to produce within the body or bodily organ/s or region/s or pathogenic region/s or diseased region/s or sub-system or loci or locus or cell/s or cellular or biological network/s etc. ‘in-vitro’ like condition/s or control or therapy or increase/s or decrease/s in the specific composition or particular element/s or compound/s or molecule/s or substance/s (e.g., including: single, multiple, assortment, combination or level/s of any single or multiple element/s, compound/s chemical/s, biological agent/s or substance/s etc.

A non-invasive or invasive subliminal or supraliminal Brain-Medic Neuro-Affective stimulator for CNS impairments or diseases or abnormalities or for enhancement of normal brain functioning comprising a system, method, medical device or apparatus or any other mechanism or mechanisms capable of providing personalized subliminal or supraliminal neuro-affective and/or neuro-cognitive stimulation and/or is geared towards or targets the treatment or healing or therapeutic improvement of any single or multiple brain- or cognitive- or affective- or behavioral or neurophysiological disease or illness/es or impairment/s or towards the enhancement of any single or multiple brain- or cognitive- or affective- or behavioral or neurophysiological function/s or capability; Such medical device comprises of the combination of non-invasive (or invasive) subliminal or supraliminal stimulus or stimuli (including but not limited to any sensory, electrical or electromagnetic or other stimulation) and/or which may be directed towards the healing or therapy or improvement of any CNS disease/s or illness/es or impairment/s or enhancement—which may be also combined with- or operate independently of any additional electrical or electromagnetic invasive or non-invasive stimulator/s which may or may not be synchronized in time-space- or space-time. The Brain-Medic invasive or non-invasive subliminal or supraliminal stimulating system may function independently of- or may also be combined with the Hope Detector or with any other diagnostic or evaluative or assessment and/or personalized system (e.g., which can operate at the same time- or convergent with- or independently of the timing of the operation of the Brain Medic stimulating device); If combined with the operation of the Hope Detector or any other assessment system (or procedure or test or measure) then the operation of the Brain Medic Subliminal or Supraliminal stimulator may be corrected or fine-tuned or adjusted continuously or intermittantly or disjoint or combined with the level/s or value/s or protocol/s of stimulation . . . .

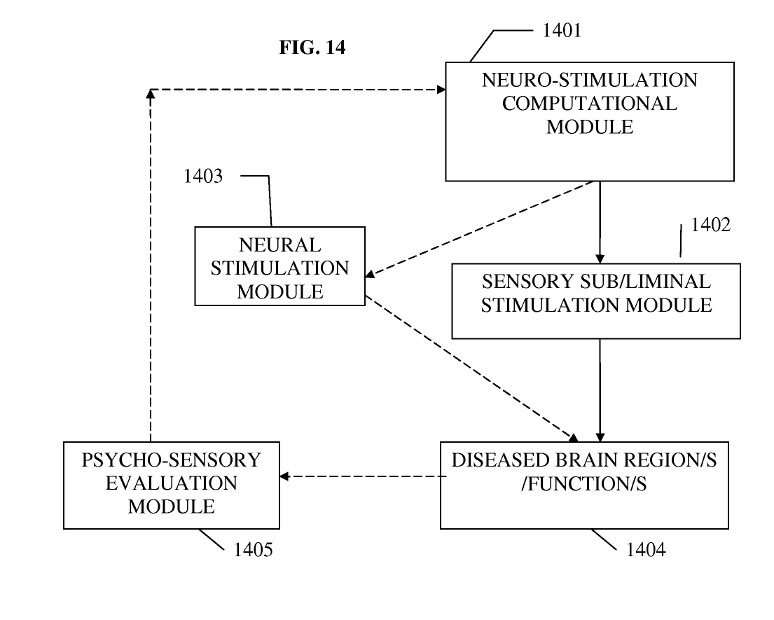

Reference is now made to FIG. 14 which describes a new Neuro-affective or Neuro-cognitive or neuro-behavioral stimulation which is geared towards treating any CNS deficit/s or disease/s or illness/es or impairment/s through either cognitive or behavioral affective or sensory stimulation and/or accompanying or convergent or synchronized or non-synchronized electrical or electromagnetic or implantable or invasive or non-invasive or cortical or sub-cortical stimulation or any combination of any of these components of neural or behavioral or cognitive or affective or neuronal or neural stimulation/s.

The invention comprises of a NEURO-STIMULATION COMPUTATIONAL MODULE 101 which is responsible for computing and controlling any specific single or multiple sensory or cognitive or behavioral or affective or neurological or neurophysiological stimulation or stimulus or stimuli that are effective in treating or improving or optimizing or enhancing any single or multiple brain or CNS or nervous system function/s or functioning etc. or disease/s or illness/es or deficiency or difficulty etc. and which can also provide personalized or collective or disease-specific or disease-state or stage- or brain impairment/s severity or dysfunction/s and which may also be adjusted and receive feedback and adjusted accordingly based on progression of disease/s or level of symptoms or degree of cognitive or behavioral or affective impairment/s etc. or may be adjusted based on improvement due to therapy or otherwise or to the operation of the Brain Medic (or any other therapeutic or pharmaceutical intervention).

The NEURO-STIMULATION COMPUTATIONAL MODULE 1401 outputs specific single or multiple SUB/LIMINAL SENSORY STIMULATION MODULE 1402 stimulus or stimuli or sensory presentation/s or stimulation/s that may be geared or target or activate or train or rewire or cause therapeutic or other positive neuroplasticity in the single or multiple brain region/s associated with the single or multiple disease/s or illness/es or brain function of CNS or nervous system deficiency or enhancement/s etc.; the SUB/LIMINAL SENSORY STIMULATION MODULE 1402 is capable of producing subliminal or supraliminal or any other sensory stimulation (which may be specific to any single or multiple brain disease/s or function/s etc. or enhancement/s etc.)—with or without convergent or synchronized or non-synchronized NEURAL STIMULATION MODULE 1403 electrical or electromagnetic or neural or neuronal stimulation which can be activated by the abovementioned NEURO-STIMULATION COMPUTATIONAL MODULE 1401, in such a way that the SUB/LIMINAL SENSORY STIMULATION MODULE 1402 may or may not be activated and produce any possible type of sensory or subliminal or supraliminal stimuli in a synchronized or non-synchronized manner with the NEURAL STIMULATION MODULE 1403 which may also include same or different brain region/s or function/s or feature/s being stimulated by the SUB/LIMINAL SENSORY STIMULATION MODULE 1402 and/or the NEURAL STIMULATION MODULE 1403—e.g., both or any one of them being activated or controlled by the NEURO-STIMULATION COMPUTATIONAL MODULE 1401. Either or both of these SUB/LIMINAL SENSORY STIMULATION MODULE 1402 and NEURAL STIMULATION MODULE 1403 are capable of producing therapeutic neuroplasticity effects through their usage of specific exercises—sensory, electrical or electromagnetic or neurophysiological or behavioral or cognitive or affective or neuronal stimulation that is known or experimented or discovered to manipulate and/or train or heal or treat the specific disease/s or illness/es or impairment/s etc. Finally, the SUB/LIMINAL SENSORY STIMULATION MODULE 1402 is capable of presenting single or multiple subliminal (e.g., including but not limited to non-perceptible) or supraliminal (e.g., including but not limited to perceptible) sensory stimulation or cognitive or behavioral or affective or neurophysiological or neural or neuronal stimulation that can be tailored or adapted or geared towards or compatible to- or specifically designed to-treat or heal or improve or produce therapeutic or positive neuroplasticity or enhance any brain function or any disease/s deficiency/ies or feature/s of cognitive or behavioral or affective or motor or any other neural function/s or trait/s or capacity that may be associated with any mental or psychological or Psychiatric disease/s or illness/es or impairment/s or associated with the desire or goal of improving or enhancing any brain related function/s or behavior or affect or cognition etc. Thus, for instance there is a whole series of possible positive and other affirmations or statements (including about self, others, significant others, attachment figures and the self's relationship or communication of affect or behavior of feelings towards them and vice versa) given in the Appendix A which describe possible such positive or therapeutic or neuroplasticity inducing statements which may be appropriate for the treatment of various CNS mental or psychological or Psychiatric illnesses or symptoms such as Depression or Anxiety or PTSD or phobias or Schizophrenia or Autism or any other brain or mental or psychological etc. condition or impairment/s (and may further be used to enhance normal brain or well-being or personality or intelligence or any other human quality or characteristic or capacity etc.)

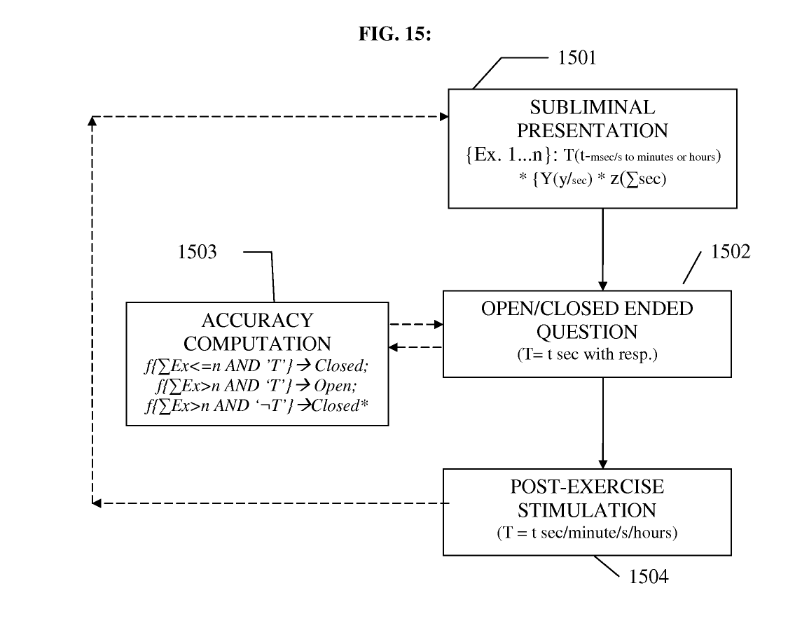

The operation of the SENSORY STIMULATION MODULE 1402 and/or of the NEURAL STIMULATION MODULE 1403 (e.g., which can be used separately or independently or in any combination—including but not limited to utilizing both modules in convergence or synchronized or non-synchronized or disjoint or intermittently etc.—for the same or different brain region/s or brain function/s including but not limited to cognitive or behavioral or affective or neural etc.) is geared towards the same or other or several or single or multiple DISEASED BRAIN FUNCTION/S 1404, which refers to any single or multiple brain region/s or cell/s or tissue/s or function/s or associated brain function/s which may be specific to a single or multiple particular disease/s or brain disease/s or brain function/s or impairment/s or deficiency/ies etc. or associated with the enhancement of normal function/s in normal individuals. Thus, the Subliminal Brain-Medic device is constructed in such a manner which allows it to selectively stimulate (subliminally or supraliminally), invasively or non-invasively any (single) or multiple diseased brain region/s or function or associated brain function/s in order to treat or improve of create therapeutic neuroplasticity effects that are specific or generalized to any given brain related disease/s or condition/s or illness or Psychiatric, psychological or mental illness or impairment/s. Note that the integrative operation of the NEURO-STIMULATION COMPUTATIONAL MODULE 1401 together with the SENSORY STIMULATION MODULE 1402 and/or the NEURAL STIMULATION MODULE 1403 which together or separately (or in any possible combination) can stimulate or heal or treat or improve or enhance the functioning or function or state/s or improve clinical condition or enhance or improve or effectively treat any CNS condition/s or deficiency or impairment/s etc. can comprise of single or multiple subliminal or supraliminal or combined or any of these stimulation element/s which can be combined with other sensory or electrical or electromagnetic or implantable or invasive or non-invasive sensory or cognitive or behavioral or affective or motor or neural or neurophysiological stimulation—all or part geared towards improving any brain or brain related or CNS or nervous or even bodily related symptom or brain function/s etc.—which may also include the flowchart FIG. 15 (below) which delineates some of the possible processing or sequence/s associated with Neuro-Affective subliminal or supraliminal stimulation of diseased brain region/s or functions of enhancement of normal brain or cognitive or behavioral or neurophysiological brain function/s;

There is also a possibility within this invention to have a ‘feedback and adjustment loop’ (or not have any such additional feedback loop—(in which case the sequence of the NEURO-STIMULATION COMPUTATIONAL MODULE 1401, SENSORY STIMULATION MODULE 1402, and/or NEURAL STIMULATION MODULE 1403 leads up to or is outputted to the DISEASED BRAIN FUNCTION/S 1404, and which may be repeated several times until a certain improvement in relevant brain function/s improve or not etc.) which comprises of the DISEASED BRAIN/FUNCTION/S 1404 prior to- during- or following- the stimulation by the SENSORY STIMULATION MODULE 1402 and/or the NEURAL STIMULATION MODULE 1403 of the DISEASED BRAIN/FUNCTION/S 1404—which is carried out by the PSYCHO-SENSORY EVALUATION MODULE 1405 which is capable of measuring any aspect or feature of the DISEASED BRAIN FUNCTION/S 1404 (e.g., before-during- or after- the stimulation by the SENSORY STIMULATION MODULE 1402 and/or the NEURAL STIMULATION MODULE 1403); Hence, the SENSORY EVALUATION MODULE's 1405 measurement of any aspect of disease/s or health or normal or above-normal or below-normal brain functioning associated with the DISEASED BRAIN REGION/S 1404 or with any particular brain related disease/s (including but not limited to neurophysiological or electrophysiological or electrical or electromagnetic or other neural measure/s or index/indices or cognitive or behavioral or motor or affective properties etc.) can occur continuously or intermittently or haphazard or random or separately or disjoint before-during- or after- the SENSORY STIMULATION MODULE 1402 and/or the NEURAL STIMULATION MODULE 1403 stimulation of the DISEASED BRAIN/FUNCTION/S 1404 is then outputted to the NEURO-STIMULATION COMPUTATIONAL MODULE 1401 which can adjust or fine-tune or alter or modulate the stimulation parameter/s or value/s or intensity/ies or frequency or behavioral or cognitive or affective or neuronal parameter/s based on the baseline or diseased or impaired or to be enhanced brain function/s prior to- during- or after stimulation (as described above); The PSYCHO-SENSORY EVALUATION MODULE can also consist of the HOPE DETECTOR (listed in this patent application) or of any other sensory, medical, psychological or psychiatric or neural or CNS evaluation system or device or method capable of diagnosing any brain related disease/s or condition/s or illness/es etc. which thereby allows the BRAIN-MEDIC SUBLIMINAL STIMULATOR (this invention) to modulate or adjust (in ‘real-time’ or ‘off-line’) any parameter/s of SENSORY STIMULATION MODULE 1402 and/or NEURAL STIMULATION MODULE 1403 stimulation parameters or protocol or cognitive or affective or motor or behavioral or neurophysiological or any other CNS stimulation value/s, feature/s or parameter/s etc.

Reference is now made to FIG. 15 which describes the flowchart diagram describing the operation of the SENSORY SUB/LIMINAL STIMULATION MODULE 1402 which therefore allows the Brain-Medic Neuro-Cognitive and/or Neuro-Affective and/or Neuro-behavioral device to stimulate the brain or CNS in such a manner which allows it to heal or treat or improve any CNS disorder/s or disease/s or illness/es or enhance any cognitive or behavioral or affective or neurophysiological or any psychiatric or behavioral or mental or psychological or couple's relationship/s or individual or couple's or organizational well-being, or any intelligence or well-being, or enhancement of individual features or qualities or executive functions or leadership or managerial or artistic or creativity or any other individual, group, organizational, family or other human function etc. or function/s through the application of subliminal or supraliminal stimulation through a SUBLIMINAL PRESENTATION 1501 which consists of presenting any subliminal or supraliminal sensory or cognitive or affective or behavioral or any other CNS stimulation (subliminally or supraliminal) for a certain period of time (which can be directed to any sensory modality or function appropriately in order to produce such subliminal or supraliminal stimulation), in conjunction with the presentation of OPEN/CLOSED ENDED QUESTION 1502 (which may also be augmented by ACCURACY COMPUTATION 1503) and the presentation of POST-EXERCISE STIMULATION 1504.

Reference is now made to the SUBLIMINAL PRESENTATION 1501 which is capable of producing any subliminal or supraliminal sensory or cognitive or behavioral or affective or neurophysiological or any other CNS such stimulation that may not be perceptible or recognized (subliminal) or may be perceptible (supraliminal) to the individual/s receiving such stimulation—but which is elicited or given in such timing and/or intensity which causes the subject to be able to process or respond or receive the relevant stimulation or sensory input (e.g., even if the subliminal stimulation is not perceptible to the individual); Hence, the specific stimulus that can be used for each subliminal stimulation/s can be tailored to each specific brain region/s or brain function/s or cognitive or behavioral or cognitive or behavioral function/s or brain- or psychological- or Psychiatric- or cognitive- or behavioral- or affective-disease/s or impairment/s etc. based on known or newly developed or discovered stimulation that can therapeutically or positively affect or improve this specific brain disease/s or impairment/s or enhance the brain or CNS or behavioral or cognitive or affective function/s or capacity/ies.

The SUBLIMINAL PRESENTATION 1501 then can comprise of any sensory or cognitive or behavioral or neurophysiological or electrical or electromagnetic or neural or neuronal stimulation and/or can consist of single or multiple such stimulus or stimuli that are presented in such a rate and/or intensity and/or duration and/or frequency that does or does not allow for a human being to be conscious of any such presentation but nevertheless be capable of processing such stimulation and allowing the brain or CNS to receive the input contained within such stimulation or to respond to any suggestion/s or stimulus or influence that such stimulation may have on the nervous system etc. Thus, for instance one possible stimulation that can comprise the SUBLIMINAL PRESENTATION 1501 are subliminal visual or auditory sentences or affirmations or stimuli which are presented for anywhere from 4 msec to 1 second (or any other time-interval etc.) at any frequency and/or intensity which is not perceptible (for subliminal stimulation) or that is perceptible (for supraliminal stimulation) and/or which can contain positive or self-affirming or body-affirming or self-image affirming or positive somatic or positive personality- or environmental- or significant other/s- or parent/s-positive attachment or parent/s' or significant others' positive rapport- or positive evaluation or regard or affection or attitude towards the individual or which may also include single or multiple sensory stimulations such as photograph/s or voice/s or other feature or association of a significant other/s or parent/s whose seen smiling or any other positive affect or perception of the parent/s or significant other/s' as positive by the individual or as lovable or as possessing a significant (positive or other) affect for the individual, and/or which may be accompanied (once or more, single or multiple times) by or associated with any single or multiple positive affirmation/s statement/s or input or affect/s or cognition or neurophysiology or electrical or electromagnetic stimulation/s or any therapeutic CNS stimulus or stimuli (including but not limited to subliminal or supraliminal stimulation). A list of such possible examples for positive affirmations in the case of Depression or Anxiety or similar or modified or same or other such principle or other affirmation/s or self- or world- or life- or other- or significant other/s- or parent/s- or attachment figure/s- or personality- or past history- or capacity to overcome any psychological or Psychiatric or CNS impairment/s statement/s or influence/s or exercise/s or stimulus/stimuli is given in an Appendix A, attached; for each single or multiple exercise comprising the SUBLIMINAL PRESENTATION 1501 there may be single or multiple presentations which can be presented sequentially or non-sequentially or combined sequentially and non-sequentially.

The rate of subliminal or supraliminal presentation can be tailored or determined of computed collectively or specifically for any brain- or behavior- or cognitive or affective- or motor- or psychological- or psychiatric- or enhancement of any individual, couple's organizational, family, executive or management or intelligence or any brain or behavior or human aspect of well-being or enhancement of function/s etc. or it may be also computed or experimented individually or personalized based on measurement/s or psychophysical or perceptual or psychological testing to determine what is the threshold below which an individual or a brain condition or disease/s may not perceive consciously the content of the text but nevertheless may be able to process or respond or encode or memorize or internalize its content/s or association/s or implication/s etc. or the rate at which there is optimization or normalization of brain or human or behavioral or cognitive or affective or neurophysiological function/s etc. (including considerations whereby “quantity may become quality”—i.e., wherein the presentation of a greater number of same or different texts or affirmation/s or (positive) statement/s may lead to significantly better brain related or psychological or Psychiatric or affective cognitive behavioral etc. results (among others due to the increased capacity to rewire or re-record positive or therapeutic or healing of enhancing messages or texts or suggestions which therefore may rewire or re-encode or “erase” negative messages etc. more rapidly or more fully or more effectively etc.)

Reference is now made to the OPEN/CLOSED ENDED QUESTION/S 1502 which refers to single or multiple question/s that may be linked or associated or otherwise convergent or non-related to the specific open or closed or other type of question/s that can appear before or after or even concurrently or synchronized or unsynchronized with the presentation of the SUBLIMINAL PRESENTATION 1501; One embodiment of the current invention consists of the presentation of the OPEN/CLOSED ENDED QUESTION/S which refer directly or indirectly to the content or association of context or ramification/s of the (same-previous- or future-) SUBLIMINAL PRESENTATION 1501 exercise/s or domains or features, including (but not limited to the possibility of) utilizing an OPEN/CLOSED ENDED QUESTION 1502 item or question/s which directly relates to the content of the previously presented exercise (e.g., such as for instance of the same Exercise 1 . . . 150, or of a previously presented SUBLIMINAL PRESENTATION 1501 exercise/s): if for instance the SUBLIMINAL PRESENTATION 1501 exercise consisted of: “I accept myself fully”, then the OPEN/CLOSED ENDED QUESTION 1502 of the same exercise or of previous or subsequent exercises may be: “how do I feel towards myself?” (open ended) or “I - - - myself - - - ” (closed ended; “correct” responses are: “accept, fully”. In such a particular embodiment the invention claims that the individual who is being presented with a subliminal (or supraliminal) assertion or self-positive statement (or other/s- or world-view- or attachment relationship/s or somatic or self-image assertions etc.) such as of the form: “I accept myself fully”—will be likely to produce a conscious response of the individual to the subsequent (same- or different-Exercise) question: “I - - - myself - - - ” (closed ended) or “I accept myself/fully” (open ended question format);

It is further claimed that a new Neuro-cognitive/affective principle entitled: “Cognitive Resonance” relates precisely the potential positive reinforcement of the subliminal (or supraliminal) suggestion/statement/s that occurs when the person responds “correctly”—i.e., in compatibility with the earlier suggestion/s—which now produces a psychological or brain related ‘urge’ or ‘tendency’ to justify or alter the person's self-image, psycho-pathology or enhance his/her self-image or positive psychological, cognitive behavioral or brain related so as to adjust to the conscious behavior/s or response/s or choice/s which the person has made as a response to the subliminal or supraliminal conditioning or priming etc. (e.g., which may be said to be somewhat opposite to Cognitive Dissonance in which the person's behavior if oppositional to his or her beliefs tends to alter these beliefs towards a greater compatibility with the observed behavior etc.) The Cognitive Resonance principle has several different potential applications and ramifications such as for instance the tendency of the person to respond in a ‘compatible response’ to the subliminal or supraliminal conditioning/stimulation and moreover once the person responds in a compatible manner or even semi-compatible manner or even in a slightly more compatible manner or tendency or frequency than his or her original response-tendency or response/s following any single or multiple (related or unrelated) subliminal or supraliminal stimulation or suggestion/s etc.; Moreover the Cognitive Resonance principle (and claim) claims that once the person has responded positively or compatibly or semi-compatibly etc. to any single or multiple subliminal or supraliminal stimulus, stimuli or suggestion/s etc. that person is much more likely to rearrange their internal belief system or response set or response/s or response likelihood in a much more compatible manner to the original subliminal or supraliminal stimulation or suggestion/s and/or to the compatible or semi-compatible or slightly more compatible response selection that the person has chosen following the subliminal stimulation/s, or to process or encode or internalize or think or feel or act or behave or respond or memorize or conceptualize information in a more compatible manner to the original subliminal/supraliminal stimulation or response selection etc. Hence, another implication or application of the Cognitive Resonance principle is that even beyond the immediate potential influence or impact of any single or multiple or even set of subliminal or supraliminal stimulation/s may have on the immediate or prospective or following single or multiple response/s or even at any later point in time (e.g., including after the immediate single or multiple Brain Medic stimulation session/s or treatment/s have commenced or any single or multiple component of these Brain-Medic stimulation/s or treatment/s such as for instance utilizing only the Neuro-Affective or Neuro-Behavioral stimulation—with or without a converging or synchronized or non-synchronized or related or unrelated stimulation by any electromagnetic or electrical or neural or neuronal stimulator (which can be a part of the Brain Medic stimulation or be carried out externally to the Brain Medic operation or function/s). Another implicit ramification and claim of the Cognitive Resonance principle is that due to the person's selection of a behavior or a response or a thought pattern/s or an emotional response/s or any cognitive or neural or behavioral or response/s set/s that is related or compatible or semi-compatible to a particular (single or multiple) subliminal or supraliminal stimulation/s or suggestion/s there is a tendency or an effect or an impact/s of such subliminal or supraliminal stimulation/s or suggestion/s and/or any subsequent response/s or selection or output/s by the individual and their immediate or prospective reorganization of their internal representation or framework or self-image or self-esteem of anti-depressive makeup or tendency/ies or evaluation which can be caused by a continuous or intermittent tendency of their neural network/s or neuronal connection/s or CNS etc. to reorient or reorganize or respond differently in such a manner that is compatible with the explicit or implicit response/s that the person has responded according to.

Additionally, the Cognitive Resonance principle is utilized when it comes to a smart feedback loop of the POST EXERCISE STIMULATION 1504 in relationship to the individual's response to the OPEN/ENDED QUESTION 1502, and/or relative to the initial or original single or multiple SUBLIMINAL PRESENTATION 1501 (e.g., assertion/s or stimulation/s or stimulus or stimuli etc.)—which are all computed by the SENSORY SUB/LIMINAL STIMULATION MODULE 1402; One possible embodiment of such potential application of the Cognitive Resonance Principle is that there is a computation or a determination or an analysis and output by the SENSORY SUB/LIMINAL STIMULATION MODULE 1402 of the precise relationship/s that may exist between the SUBLIMINAL PRESENTATION 1501 of any single or multiple statement/s or assertion/s or suggestion/s or stimulus or stimuli and the immediate, prospective or subsequent or any-spatial-temporal response of the individual to the precise content of the answer to a question/s that is/are somehow related to this single or multiple subliminal or supraliminal stimulation/s: thus, for instance if the subliminal or supraliminal stimulation stated: “I believe in myself fully” and a subsequent single or multiple Closed Ended question/s asked: “I - - - in myself - - - ” and the person responded: I do not believe in myself at all” then the SENSORY SUB/LIMINAL STIMULATION MODULE 1402 would likely compute and output a POST-EXERCISE STIMULATION 1504: “I am open to believing in myself”—rather than the more emphatic post-stimulation statement: “I believe in myself fully”—precisely based on the Cognitive Resonance principle which attempts to increase the cognitive compatibility that exists between the person's responses to any subliminal or supraliminal pre-stimulation or post-stimulation assertion/s or suggestion/s etc.

In fact the Cognitive Resonance principle serves as a major anchor or structure within the BRAIN-MEDIC NEURO-AFFECTIVE/COGNITIVE device or invention as it is reinforced several times—such as in the ACCURACY COMPUTATION 1503 contingency computation determining whether an open or closed ended question format will be utilized or other question format will be used, as well as in the incorporation of a POST-EXERCISE STIMULATION 1504 which essentially reiterates the same or similar or enhancement of the original (same- or different-Exercise message/s)—but which nevertheless monitors and computes the compatibility difference between the person's response/s and the original subliminal/supraliminal message/s, as given the example above.

Below are outlined some of the key operation principles of the Neuro-Cognitive/Affective stimulation which are designed to treat or heal or improve any CNS or couple's or depression or any other mental illness or Psychiatric illness or cognitive or behavioral or affective or human intelligence or well-being etc. capacities or capabilities etc.

Principles of Operation:
1) Each individual receives a certain amount of Neuro-Affective/Cognitive stimulation (e.g., again with or without accompanying electrical or electromagnetic stimulation for a certain amount of time daily such as for instance 35-45 minutes of Neuro-Affective Stimulation (daily).
2) The Neuro-Affective stimulation may consist of several different Clusters (grouped according to content or disease/s or impairment/s etc.—such as for instance two Clusters (#1 & #2)—with each of these Clusters also comprised of several Groups of exercises which may be aimed at different CNS disease/s or impairment/s etc.; Each group consists of multiple Neuro-Affective Exercises (e.g., all of which each of the couple's partners receives).
3) Each Neuro-Affective Exercise is presented for a ‘x’ length of time (such as for instance x=4-8 msec′) a certain ‘y’ number of times per second (for instance: y=6/sec) for a ‘z’ number of seconds (e.g., for instance approximately 4 seconds—calculated by 60 minutes×60 sec′=3600 sec′/350 Exercises*=10.28 sec minus—out of which 4 sec′ for Open/Closed ended question+2 sec′ Post-Exercise Stimulation, =6 sec′, which leaves us approx. 4 seconds (‘z’) for presenting each Subliminal Stimulation for each of the Neuro-Affective Exercises.
4) Note that there the total of approximately 350 Exercises consists of two Clusters of Neuro-affective Exercises: e.g., Cluster #1 with 167 Exercises and Cluster #2 with only 90 Exercises—which are each presented twice (if possible non-consecutively)=180 Exercises for Cluster #2.
5) Following the completion of the series (y×Σsec) of presentations of each individual Neuro-Affective Exercise there appears an immediate closed-ended question (which is listed for each Exercise), with the person keying in their response for each Subliminal Stimulation interval of each exercise.
6) *Each Neuro-Affective Exercise should be adapted to the sex of the subject.
7) Following the subject's response there appears a 1 second ‘Post-Exercise Stimulation’ text which is then followed by the next sequence of the next Neuro-Affective Exercise (see FIG. 1 below).
8) Following a three-days consecutive ‘correct responses’ (e.g., responses which are compatible with the Neuro-Affective stimulation) on any given item the question format will change to ‘open ended’ (e.g., also listed for each question); However, if after shifting to an open-ended question format for a particular question (e.g., after three consecutive days in which a subject has responded correctly on that particular question/s) the subject responds incorrectly on any three days (e.g., which may also be non-consecutive), then the question format will revert back to a closed-ended format for the remainder of that subject's treatment duration.
9) For each question in which the person is asked about themselves (alone) then a picture of that individual which he/she likes (e.g., preferably smiling or happy) needs to be presented alongside the Neuro-Cognitive Exercise (for the duration of time ‘x’).
10) For each question relating to Father/Mother—instruct individual to supply photograph of the most loved parent;
11) For each question in which the person is asked about their (loved) parent or (loved) partner then a picture of that loved parent or partner which the individual likes (e.g., preferably expressing love towards the individual) needs to be presented alongside the Neuro-Cognitive Exercise (for the duration of time ‘x’=4-8 msec).
12) For each of the Neuro-Affective Exercises in which there are more than 3 words in the subliminal presentation the text has to be presented in two lines

1) Appendix: The Computational Unified Field Theory (CUFT) or Unification between Quantum Mechanics and Relativity Theory and Other Computational Issues

The ‘Duality Principle’

The contradiction between quantum mechanics and Relativity Theory constitutes perhaps the most striking theoretical enigma in modern natural sciences. Indeed, the principle reason for this conceptual schism between these two theoretical frameworks stems from Relativity's positivistic vs. quantum's only probabilistic predictive characteristics. But, it is hereby hypothesized that a common (novel) computational ‘Duality Principle’ potentially constrains both Relativity's assumed (computation of) the constancy of the speed of light, as well as the probabilistic interpretation of quantum mechanics—based on its identification of a common ‘self-referential ontological computational’ [SROCS] structure underlying both theoretical paradigms. Succinctly stated, the Duality Principle proves that it is not possible (in principle) to determine the ‘existence’/‘non-existence’ of a hypothetical ‘y’ element from within its direct physical interaction with another ‘x’ element (termed: ‘di1’); This is because such SROCS structure: OCP/di1=PR{x,y}→[y or [symbol] y] includes the possibility of a ‘self-referential ontological negative statement’ [SRONCS] of the structure: OCP/di1=PR{x,y}→ [symbol] y, which inevitably leads to both ‘logical inconsistency’ (e.g., the ‘y’ element is asserted to both ‘exist’ and ‘not exist’ at the same computational level ‘di1’) and ‘computational indeterminacy’ (e.g., ensuing inability to determine whether the ‘y’ element ‘exists’ or does ‘not exist’ at this ‘di1’ computational level). However, given robust empirical evidence for the capacity of both quantum and relativistic computational systems to determine the existence/non-existence of the ‘y’ value (i.e., the constancy of the speed of light or the complimentary energetic/spatial or temporal/mass 1 values of the subatomic ‘target’), the Duality Principle infers that the determination of the existence/non-existence of the ‘y’ element (termed: ‘ontological computational processing, ‘OCP’) must take place at a conceptually higher-ordered ‘D2’ computational level (e.g., that is in principle irreducible to the direct physical interaction between the ‘x’ and ‘y’ entities at ‘di1’): OCP/D2→[‘y’ or ‘ [symbol] y’]≠PR{x,y}/di1

Specifically, the Duality Principle challenges Relativity's (assumed) capacity to determine the constancy of the speed of light from within the direct physical interaction between an observer and a beam of light, as well as quantum mechanics' probabilistic interpretation of the uncertainty principle; This is because the Duality Principle points at the fact that both (computational) paradigms create the abovementioned SROCS structure which leads to logical inconsistency and computational indeterminacy, which (in turn) are contradicted by the capacity of relativistic and quantum systems to determine the constancy of the speed of light as well as the complimentary (spatial-energetic or temporal-mass) values of the subatomic target element; Hence, the ‘Duality Principle’ asserts the conceptual computational inability to determine the ‘existence’ or ‘non-existence’ of the constancy of the speed of light {‘c’ or ‘ [symbol] c’} from within the direct physical interaction between a relativistic observer and the light beam, as well as the conceptual computational inability to determine the complimentary (spatial/energetic or temporal/mass) value of the subatomic target from within its direct physical interaction with another probing element.

In the case of the relativistic determination of the speed of light the resulting SROCS may be formalized thus: SROCS: OCP/di1=PR{Odiff, c}→[‘c’ or ‘ [symbol] c’] wherein the constancy of the speed of light is determined based on its direct physical interaction with a (relativistic) observer (at the ‘di1’ level). However, computationally such SROCS structure possesses the potential to lead to a SRONCS output thus:

OCP/di1=PR{Odiff, c}→‘ [symbol] c’, wherein the direct physical interaction between a relativistic observer and a beam of light (e.g., traveling at the speed of light)—may lead to a computation of a light-beam not traveling at the speed of light, which would obviously result in both logical inconsistency and computational indeterminacy . . . . But, as we know relativistic (computational) systems are capable of determining the (constancy of the) speed of light, thereby proving the Duality Principle's insistence upon placing OCP→[‘c’ or ‘ [symbol] c’] at a conceptually higher-ordered D2 level;

Note that the empirical fact that the speed of light is always measured at ‘c’ does not in any way negate the SROCS/SRONCS computational structure, nor the Duality Principle's resulting conceptual computational higher-ordered placement of OCP at D2.

This is because the SROCS structure of Relativity's assumed constancy of the speed of light computationally possesses the potential of outputting a SROCS result, e.g., OCP/di1=PR{Odiff, c}→‘ [symbol] c’, which inevitably leads to both logical inconsistency and computational indeterminacy—both of which are negated by empirical relativistic measurements indicating the capacity to measure the constancy of the speed of light (by any relativistic observer); Hence, from an empirical-computational standpoint the Duality Principle challenges Relativity's assumed capacity to determine the constancy (e.g., or lack of constancy) of the speed of light from within the direct physical interaction between an observer and a beam of light (at the ‘di1’ computational level). Instead, the Duality Principle points at the existence of a conceptually higher-ordered OCP/D2 which alone can carry out the necessary computation determining the constancy/ [symbol] constancy of the speed of light (e.g., rather than through any hypothetical interaction between a relativistic observer and a light beam). Apparently, one of the defining empirical characteristics of such OCP/D2 is its consistent determination of the constancy of the speed of light (which would therefore be incorporated later on as an ‘objective’ empirical-computational constraint of OCP/D2).

In the case of the probabilistic interpretation of quantum mechanics the SROCS/SRONCS structure may be represented thus: OCP/di1=PR{p,t}→[‘t’ or ‘ [symbol] t’], wherein the direct physical interaction between a subatomic target (e.g., which is assumed to be dispersed all along a probabilistic wave function) and another probe elements is believed to determine the ‘existence’ or ‘non-existence’ of the target element at one of the possible (complimentary energetic-spatial or temporal-mass) localizations along this probability wave function. But, if the existence/non-existence of the subatomic target element at any given complimentary value; ‘t[s/e or t/m]i1 . . . n’ or ‘ [symbol] t[s/e or t/m]i1 . . . n’] is allowed to take place at the same computational level at which the direct physical interaction [‘di1’] between these two subatomic elements takes place then this can lead to the abovementioned logical inconsistency and computational indeterminacy (which inevitably result from a SRONCS structure):

OCP/di1=PR{p[s/e or t/m]i1, t[s/e or t/m]i1 . . . n}→ [symbol] t[s/e or t/m]i1, wherein the direct physical interaction between the target element (which is assumed to be dispersed all along the probability wave function's complimentary spatial/energetic or temporal/mass possible points) and the subatomic probe—leads to a situation in which the target element is computed (e.g., presumably at OCP/di1) to not exist at all t[s/e or t/m]i1 . . . n except the one measured t[s/e or t/m]i1 complimentary value. However, such SRONCS computational output, inevitably leads to logical inconsistency and computational indeterminacy which are contradicted by quantum system's capacity to determine the existence or non-existence of the subatomic target element at a particular (complimentary) t[s/e or t/m]i1 . . . n localization. Hence, the Duality Principle asserts the conceptual computational inability to determine the existence/non-existence of the subatomic target from within the direct physical interaction (di1) between the subatomic target and probe elements, but only at a conceptually higher-ordered ‘D2’ level.

Therefore, the Duality Principle asserts the conceptual computational irreducibility of OCP determining the existence/non-existence of ‘t[s/e or t/m]i1’ or of ‘c’ to within their direct physical interaction with any ‘p [s/e or t/m]i1’ or ‘Odiff’ (respectively). However, such conceptual computational inability to determine the existence/non-existence of the target particle at a particular complimentary (spatial/energetic or temporal/mass) point along a hypothetical probabilistic distribution of the wave function—as a result of the direct physical interaction between the subatomic probe and target elements directly challenges the current probabilistic interpretation of quantum mechanics. This is because the probabilistic interpretation of quantum mechanics assumes that the subatomic target element is distributed all along the (complimentary) probability wave function—up until it directly interacts with another probing element, which then determines (i.e., ‘collapses’) the probability wave function into a single complimentary (spatial/energetic or temporal/mass) point . . . . But, if the Duality Principle's proof for the conceptual computational inability to determine the complimentary (spatial/energetic or temporal/mass) ‘whereabouts’ of the subatomic target—from within its (direct) physical interaction with the probe element is accepted, then we must also give up its associated probabilistic interpretation of quantum mechanics wherein the target element is assumed to be dispersed probabilistically (e.g., along a probability wave function) prior to its interaction with the probe element. Instead, what we're left with are only the empirical findings regarding the complimentary spatial/energetic or temporal/mass value of any subatomic ‘target’ particle—that nevertheless may only be determined through a conceptually higher-ordered (and irreducible) OCP/D2 computational level.

Hence, the Duality Principle clearly points at the conceptual computational irreducibility of both Relativity's constancy of the speed of light and quantum's probabilistic interpretation of the uncertainty principle—to within their (currently formulated) direct physical interaction between a light-beam and the relativistic observer or between the subatomic target and any hypothetical subatomic probing element. Instead, it advocates the search for a conceptually higher-ordered OCP/D2 as the only possible computational model that can potentially account for both relativistic space-time as well as quantum phenomenology (e.g., based on the recognition of the ‘un-feasibility’ of either Relativity's or quantum's current SROCS computational models which inevitably lead to logical inconsistency and computational indeterminacy that are contradicted by robust empirical evidence). As such, it also opens the door for Einstein's long-sought for unifying (field) theory which could be based upon a singular hierarchical-dualistic computational principle. This is because if indeed, the only possible OCP/D2 of both Relativity's space-time configuration and of quantum's complimentary energy-space and temporal-mass values may be derived from a conceptually higher-ordered computational level (e.g., that is in principle irreducible to the direct ‘observer-event’ relativistic or probe-target’ quantum physical interaction ‘di1’), then it is reasonable to expect that we should be able to formulate such a singular conceptually higher-ordered D2 computational framework upon which to base the empirical findings of Relativity and quantum observations;

Characterizing Quantum vs. Relativistic Computation

Hence, the basic stipulation of such (conceptually) higher-ordered OCP/D2 computational framework is that there exists such irreducible (singular) computational framework that determines the empirical value of both relativistic and quantum measurements. What follows is an analysis of the key computational features of such a tentative conceptually higher-ordered OCP/D2, e.g., which may satisfy (singularly) both quantum and relativistic empirical data;

Positivistic vs. Probabilistic Computation.

First, I suggest that the Duality Principle's challenging of the (current) SROCS structure underlying both the probabilistic interpretation of the quantum uncertainty principle and the relativistic (assumed) constancy of the speed of light may circumvent the (above mentioned) schism that seems to exist quantum mechanics and Relativity theory; This is because a fuller analysis of the theoretical ramifications of the Duality Principle may indicate that it necessitates a reformalization of the conceptually higher-ordered hierarchical-dualistic computational reality which transcends both the probabilistic interpretation of quantum mechanics and the positivistic conceptualization of Relativity Theory. Hence, as outlined above, the Duality Principle challenges not only the conceptual inability to determine the complimentary (spatial/energetic or temporal/mass) value of a subatomic target particle based on its direct physical interaction with another probing element—but more significantly undermines the theoretical validity of the hypothetical existence of a probability wave function along which the subatomic target particle is assumed to be dispersed (e.g., prior to its direct physical interaction with the probe element). But, if indeed the Duality Principle undermines the probabilistic distribution of any hypothetical target particle (prior to its direct physical interaction with another probe element), then the tentative hierarchical-dualistic computational framework may only be characterized through the (above mentioned) empirical facts of quantum complimentarity, e.g., but not as characterized by any probabilistic (wave function) distribution.

In much the same manner, I hypothesize that the Duality Principle's proof for the conceptual inability to determine the constancy of the speed of light from within the direct physical interaction between any (hypothetical) relativistic observer and a beam of light may point at a non-positivistic (reductionistic) hierarchical-dualistic computational framework; This is due to the fact that the lack of an ability to determine the constancy of the speed of light from within the direct physical interaction (‘di1’) between an observer and a beam of light—also has (potentially) far reaching computational ramifications which may be characterized as: ‘computational-relativistic uncertainty’. This is because each of the four basic physical properties of space, time, energy and mass is determined (according to Relativity theory) based on the direct physical interaction between an observer and a constant speed light-beam. But, since the Duality Principle asserts that it is not possible to determine the constancy of the speed of light from within the direct physical interaction between an observer and a beam of light, then from a (purely) computational standpoint we obtain a mutual contingency of complimentary computational pairs, e.g., space and energy or mass and time—which together yield the constancy of the speed of light unified value! As a matter of fact, I suggest a perceptual-computational metaphor which may assist us in explicating such hypothetical complimentary computational uncertainty between an object (or event's) space and energy or mass and time values: Imagine a condition in which a (relativistic) observer is devoid of the capacity to measure the contextual environment of a source of light which is (rapidly) approaching it (e.g., such as for example in a ‘dark’ chamber which does not allow for any light-signal measurement of the distance or velocity of this approaching object or event). In such a situation we can immediately see that there is no conceptual possibility to distinguish between a distant yet relativistically fast approaching object and a near object which approaches the observer relatively slowly! Note that such hypothetical complimentary spatial-energetic uncertainty could arise as a result of the (above mentioned) Duality Principle's assertion regarding the conceptual inability to determine the constancy of the speed of light from within the direct physical interaction between any relativistic observer and a light beam. This is because if (as the Duality Principle claims), it is not possible to determine the constancy of the speed of light from within the direct physical interaction between a beam of light and a relativistic observer (e.g., at ‘di1’—but only at a conceptually higher-ordered OCP/D2) then we cannot utilize the constancy of the speed of light as a means of determining whether the rapidly approaching observer is travelling fast but is distant or traveling slow but is closer to the observer . . . .

But, since (again based on the Duality Principle's conceptual proof) there exists empirical evidence indicating that relativistic systems are capable of determining the constancy of the speed of light as well as its associated space-time, energy-mass values etc. we must conclude that there exists a conceptually higher-ordered OCP/D2 which alone is capable of computing an object's (or event's) relativistic (as well as quantum) values. Nevertheless it is worth noting that such (potential equivalent) relativistic computational complimentarity may (later on) point at a computational connection that exists between the two pairs of basic physical properties, e.g., space and energy, time and mass—which is independent of any (current) probabilistic-quantum or relativistic theoretical frameworks . . . . Indeed, what follows is a delineation of an important distinction between the Duality Principle's negation of the existent theoretical models underlying Relativity theory and (probabilistic) quantum mechanic, and the actual empirical data—which therefore should set factual constraints upon the hypothetical conceptually higher-ordered OCP/D2 framework that can alone (singularly) determine both the quantum and relativistic values of an object or event;

Empirical Facts vs. Theoretical Interpretations

Indeed, I suggest that one of the key changes brought about by the Duality Principle's abovementioned challenges to relativistic and quantum computational modeling involves a rethinking of empirically validated measurements vs. (currently) assumed theoretical constructs underlying both quantum and relativistic models. Thus, for instance based on the Duality Principle's proof for the conceptual computational inability to carry out the (necessary) OCP/D2 at either the (relativistic) observer-event interaction or at the (quantum) probe-target subatomic interaction (‘di1’)—we are forced to abandon both relativistic and quantum (existent) models in favor of a purely empirical account of their (respective) measured effects; This implies that the only incontrovertible empirical facts that we may consider as indispensible (across both relativistic and quantum realms) are: the constancy of the speed of light e.g., as computed by a conceptually higher-ordered OCP/D2 for any hypothetical spatial-temporal point, and the complimentarity of both quantum and relativistic spatial-energetic, and temporal-mass physical computation, e.g., again as computed solely through a conceptually higher-ordered OCP/D2. In contrast, the superimposed theoretical constructs arising from Relativity's assumption wherein it is possible to determine the space-time (energy-mass or gravitational curvature etc.) of an object (or event) based on the direct physical interaction between a beam of light (emanating from an event) and an observer must be rejected (based on the abovementioned Duality Principle computational constraints). Similarly, I hypothesize that (current) quantum theoretical superimpositions consisting of the (abovementioned) probabilistic interpretation e.g., wherein the subatomic target is assumed to be dispersed all along a probability wave function up until its direct physical interaction with the subatomic probe—must be rejected.

Where does these lead us, e.g., in terms of trying to construct an alternative hierarchical-dualistic computational model (as demanded by the Duality Principle) of these raw empirical relativistic and quantum facts?

I suggest that a deeper examination of the common computational grounds underlying such hierarchical-dualistic computational reformalization of relativistic and quantum modeling may point at the existence of a singular ‘hierarchical-dualistic computational framework’, e.g., a (singular) conceptually higher-ordered OCP/D2 computational framework which (alone) can determine the existence/non-existence of any hypothetical relativistic or subatomic spatial-temporal (complimentary) object or event (which is in principle irreducible to the direct physical interaction (‘di1’) between any subatomic probe ad target elements or relativistic direct physical interaction between an observer and a beam of light emanating from any given object or event).

I suggest that the key importance of such hypothetical (singular) hierarchical-dualistic computational framework is twofold: in its insistence on the total contingency of all spatial-temporal quantum as relativistic phenomena upon a conceptually higher-ordered (irreducible) computational framework (OCP/D2), and the necessity of unifying both quantum and relativistic models as originating from- (and being constrained by-) such conceptually higher-ordered computational framework! Finally, I also suggest that this hypothetical hierarchical-dualistic computational reality should nevertheless be itself constrained by the (abovementioned) incontrovertible empirical facts found thus far in quantum mechanics and relativistic measurements.

‘The Hierarchical-Dualistic Computational Reality’

How does one go about proving the existence of this hypothetical hierarchical-dualistic computational reality (HDCR)? Well, perhaps the most direct way of proving the existence of the HDCR is based on the earlier mentioned conceptual computational inability to determine the ‘existence’ or ‘non-existence’ of any (hypothetical) spatial-temporal relativistic as well as quantum (complimentary) object or event—from within its direct physical interaction with any other (hypothetical) relativistic observer or subatomic probe element. This implies that our (ontological) knowledge of any relativistic or quantum object/event—or indeed the very fabric of space-time, energy-mass, or quantum complimentary particle/wave measurements—may only be determined based on a conceptually higher-ordered OCP/D2 that is (again in principle) irreducible to the direct observer-event relativistic or subatomic probe-target physical interactions. But, I claim that this basic contingency of all our (ontological) knowledge of all relativistic or quantum object/events upon a conceptually higher-ordered OCP/D2 (that is irreducible to the ‘di1’ relativistic or quantum interactions) precisely defines the abovementioned HDCR! The essence, therefore of the HDCR is its conceptual assertion that the totality of all quantum or relativistic physical phenomena—may be known to ‘exist’ solely on the basis of a conceptually higher-ordered computational framework OCP/D2 (which itself is in principle irreducible to the ‘di1’ quantum or relativistic physical interactions) . . . .

The philosophical significance of the HDCR is (off course potentially) enormous, because it challenges (and undermines) the current (largely) ‘materialistic-reductionistic’ ontological assumption underlying most scientific physical paradigms; It implies that the ‘physical’ universe in which we live can only be known to us via conceptually higher-ordered non-materialistic computational processes (OCP/D2).

Moreover, as I will try to prove now, the singularity of such HDCR as underlying- and constraining- both quantum and relativistic frameworks brings us that much closer to their potential unification within one conceptually higher-ordered OCP/D2: Suppose there exist two ‘separate’ ‘OCP/D21’ and ‘OCP/D22’ as underlying and constraining quantum and relativistic modeling (e.g., which is somewhat reminiscent of the current conceptual rift between quantum and relativistic models). Then, according to the Duality Principle this would imply that in order to be able to determine any hypothetical physical relationship between quantum [‘qi{1}’] and relativistic [‘ri{2}’] entities or processes—we would necessarily need a conceptually higher-ordered ‘OCP/D3’ that is (again in principle) irreducible to the lower-ordered OCP/D2[‘qi{1}’, ri{2}’] ‘di1’ physical interactions. This is because otherwise, the determination of the ‘existence’ or ‘non-existence’ of any such hypothetical quantum or relativistic phenomena would be carried out at the same computational level (‘D2’} as the direct physical interaction between these (hypothetical) quantum and relativistic entities (or processes), thereby precisely replicating the SROCS structure (that is constrained by the Duality Principle), thus: OCP/D2=PR[‘qi{1}’, ri{2}’]→[(qi{1}, ri{2}) or [symbol] (qi{1}, ri{2})].

But, since we already know that the Duality Principle proves the conceptual computational inability to carry out OCP at the same computational level (e.g., in this case termed: ‘D2’) as the direct physical interaction between the given elements, then we are forced (once again) to conclude that there must be only one singular conceptually higher-ordered OCP/D2 computational level underlying both quantum and relativistic models. Therefore, we are led to the (inevitable) conclusion whereby there may only exist one conceptually higher-ordered OCP/D2 as underlying and constraining both quantum and relativistic phenomenology.

Single- vs. Multiple-Spatial-Temporal Measurements

Perhaps a direct ramification of the above mentioned critical difference between empirical facts and theoretical modeling which may have a direct impact on the (current) schism between Relativity Theory and quantum mechanics is the distinction between single- vs. multiple-spatial-temporal empirical measurements and its corresponding ‘particle’ vs. wave’ theoretical constructs; I hypothesize that if we put aside (for the time being) the positivistic vs. probabilistic characteristics of Relativity theory and the probabilistic interpretation of quantum mechanics (e.g., which were challenged by the Duality Principle above), then we may be able to characterize both relativistic and quantum empirical data as representing single- vs. multiple-spatial-temporal measurements! Thus, for instance, I suggest that whereas a (subatomic) particle or (indeed) any relativistic object or event can be characterized as indicating a single (localized) spatial-temporal measurement such that the given object/event is measured at a particular (single) spatial point {si} at any given temporal point {ti}, the wave characteristics of quantum mechanics represent a multi spatial-temporal measurement wherein there are at least two separate spatial-temporal measurements for each temporal point {siti, s(i+n)t(i+n)}.

Indeed, I hypothesize that precisely such a distinction between single- and multiple-spatial-temporal measurement (and conceptualization) may stand at the basis of some of the quantum conundrums such as the particle-wave duality, the double-slot experiment, and quantum entanglement; Specifically, I suggest that if (indeed) the primary difference between the ‘particle’ and ‘wave’ characterization is single- vs. multiple-spatial-temporal measurements, then this can account for instance for the (apparently) ‘strange’ empirical phenomena observed in the double-slot experiment. This is because it may be the case wherein the opening of a single slot only allows for the measurement of a single spatial-temporal measurement at the interference detector surface (e.g., due to the fact that a single slot opening only allows for the measurement of the change in a single photon's impact on the screen). In contrast, opening two slots allows the interference detector surface to measure two spatial-temporal points simultaneously thereby revealing the ‘wave’ (interference) pattern. Moreover, I hypothesize that if indeed the key difference between the particle and wave characteristics is their respective single- vs. multiple-spatial-temporal measurements, then is may also be the case wherein any ‘particle’ (e.g., or for that matter also any single spatial-temporal relativistic measurements) is embedded within the broader multi-spatial-temporal ‘wave’ measurement . . . . In this case, the current probabilistic interpretation of quantum mechanics (which has been negated earlier by the Duality Principle) may give way to a hierarchical computational interpretation which regards any particle measurement as merely a localized (e.g., single spatial-temporal) segment of a broader multi spatial-temporal ‘wave’ measurement!

Empirical Characterization of the HDCR:

The key question is therefore: how may we characterize this HDCR which solely underlies and constrains both quantum and relativistic empirical findings?

I suggest that we can accurately characterize this HDCR through the combination of examining the (abovementioned) raw empirical facts offered by quantum and relativistic measurements, coupled with an analysis of the convergent computational characteristics emerging from these (apparently divergent) quantum vs. relativistic traits;

First (as indicated earlier) what we're left with in terms of the raw empirical facts of quantum and relativistic measurements (e.g., stripped from their respective theoretically superimposed constructs) is threefold: the constancy of the speed of light for all relativistic observers (i.e., but which is computed at a conceptually higher-ordered OCP/D2 for each hypothetical spatial-temporal observer-event ‘di1’ physical interaction); The distribution of quantum subatomic measurements along a probability wave function (e.g., but which does not postulate the distribution of the target element along such probability wave function neither assumes that the computational ‘collapse’ of such a probabilistically distributed target element into a single ‘particle’ measurement depends upon its direct physical interaction with another subatomic probing element); The existence of apparent single spatial-temporal ‘particle’ elements vs. multi spatial-temporal ‘wave’ subatomic measurements that seem to co-exist (often interchangeably); And the complimentarity of spatial/energetic and temporal/mass measurements in quantum mechanics and (as been shown above) potentially also in relativistic computation.

When viewed from the perspective of the conceptually higher-ordered OCP/D2 these raw empirical findings may set the basic ad hoc parameters upon which the HDCR must be based; Even though the constancy of the speed of light was shown to be stripped of its direct (physical interaction) determination of an event's space-time (energy-mass) value (e.g., instead being replaced by a conceptually higher-ordered OCP/D2 determination of an observer-light physical relationship)—the empirical constancy of the speed of light must remain as an objective dictum constraining any such conceptually higher-ordered OCP/D2. Similarly, despite the significant alteration of (the current) probabilistic interpretation of quantum mechanics (e.g., based on a rejection of the assumption wherein the quantum target particle is dispersed probabilistically along a wave function until its direct physical interaction with another probing element)—the empirical complimentarity of spatial/energetic and temporal/mass measurements along with the quantum measurements of a particle element along a probability wave function must likewise inform the constraints of this novel OCP/D2 HDCR.

So, the key inquiry becomes one that attempts to embed (or construe) these incontrovertible empirical (quantum and relativistic) facts within the hypothesized HDCR—based on an analysis of OCP/D2 (novel) reformalization of quantum and relativistic computational principles. I therefore intend to reexamine the HDCR's essential characterization of the central computational principles underlying (and constraining) the abovementioned raw empirical quantum and relativistic data. Indeed, I will try to evince that such HDCR computational analysis may point at the convergence of quantum and relativistic computational principles within such (mutual) OCP/D2 computational framework;

Computational Irreducibility and Simultaneity:

Perhaps the key to unraveling the computational characteristics of this tentative HDCR is found within its (core) ‘computational irreducibility’ inherent in its hierarchical-dualistic framework (e.g., the Duality Principle); Remember that earlier we've seen that in both quantum and relativistic realms, the Duality Principle proves the conceptual computational irreducibility of either the relativistic space-time (energy-mass, gravitational curvature, etc.) properties of an object (or event), or the complimentary (space/energy or temporal/mass) subatomic features of a target—based on the direct physical interaction between an observer and an event or between a subatomic probe and target elements, but only by a conceptually higher-ordered OCP/D2 which is (in principle) irreducible to the ‘di1’ relativistic or quantum (interactive) levels. This implies that only computational means of determining the existence/non-existence of any space-time, energy-mass, gravitational curvature etc. object/event or of any subatomic quantum complimentary spatial/energetic or temporal/mass particle or event may only be determined by a conceptually higher-ordered OCP/D2 (but not through any di1 observer-event or probe-target interactions).

Thus, for instance I hypothesize that the determination of the simultaneity of any two hypothetical spatial-temporal events may not be computed by a (relativistic) observer (e.g., as this would inevitably produce the SROCS computational structure constrained b the Duality Principle), but only by the higher-ordered HDCR. Instead, the determination of the simultaneity or non-simultaneity (between two hypothetical events) may only be carried out by the conceptually higher-ordered hierarchical-dualistic computational reality (OCP/D2) that is (in principle) irreducible to the direct physical interaction between the light beams (travelling from the two events) to the relativistic observer. But, if indeed the determination of the simultaneity of any two hypothetical events may not be determined by any (spatial-temporal) relativistic observer which interacts with these two events via a light-beam signal traveling from them to the observer, then this implies that the higher-ordered HDCR (OCP/D2) computation of simultaneity must necessarily not be constrained by the speed of light! Obviously, the implications of the independence of OCP/D2's determination of the simultaneity (or non-simultaneity) of any two hypothetical events may be quite far reaching—as it undermines Relativity's current formalization which regards the speed of light as a universal physical limit set on the transference of any information or physical effects across space. Indeed, one of the key points at which relativistic and quantum theories clash is precisely related to empirical quantum findings of entanglement which indicate that (subatomic) information can be transferred across distances greater than afforded by the light travelling distance between two (entangled) events . . . . However (as noted above), since HDCR indicates that OCP/D2's determination of the simultaneity (or non-simultaneity) of any two given physical events is not constrained by the speed of light (e.g., nor the direct physical interaction between light signals traveling from these two hypothetical events to a relativistic observer), then this affords an alternative hierarchical-dualistic (relativistic) theoretical framework that may account for the empirically validated entanglement quantum findings. As a matter of fact, given the fact that HDCR asserts the conceptual computational inability to compute simultaneity/non-simultaneity based on the direct physical interaction between a light beam/s and a relativistic observer (but only from a conceptually higher-ordered OCP/D2), the role of the speed of light necessarily changes from the direct measurement of the spatial-temporal localization of an object (or an event)—to a computational tool for the higher-ordered OCP/D2 (which in principle does not utilize the direct physical interaction ‘di1’ between the light signal emanating from an object/s or an event/s and the relativistic observer as a means for determining in this case the simultaneity/non-simultaneity of any two hypothetical events). In this respect, quantum entanglement represents one case study indicating that the speed of light does not serve as a limit for computation of the spatial-temporal OCP/D2 (or as shown above—actually for any such conceptually higher-ordered OCP/D2). Therefore I suggest that the constancy of the speed of light (and the computational role it may play in the higher-ordered OCP/D2) should be considered (again) as a purely empirical finding that needs to constrain the physical parameters of the hypothetical HDCR, but which should be stripped of any previous relativistic theoretical considerations.

Universal Simultaneous Computational Frame (USCF)

Thus, it seems that the Duality Principle's conceptual computational inability to determine the simultaneity/non-simultaneity based on the direct physical interaction between light-beam(s) emanating from two hypothetical events and a relativistic observer (on the one hand)—which is contrasted with the proven empirical capacity to determine simultaneity of two (relativistic) events necessarily leads to a reassertion of the HDCR's determination of simultaneity only at the conceptually higher-ordered OCP/D2. Indeed, the case of quantum entanglement precisely proves this conceptual inability of (two) light (beams) which emanate from two hypothetical spatial-temporal events and interacting directly with a relativistic observer—to determine their physical relationship (e.g., including questions of ‘simultaneity’ or ‘non-simultaneity’). We are thus led to conclude that (a) it is clear that the determination of the ‘simultaneity’ or ‘non-simultaneity’ of two hypothetical events may not be determined through their (light-signal's) direct physical interaction with an observer, but only at the higher-ordered OCP/D2 (constituting the HDCR); and (b) that since empirical measurements do indicate the capacity to determine simultaneity or non-simultaneity of two such hypothetical events, e.g., such as in the case of quantum entanglement then we must conclude: (c) the conceptually higher-ordered OCP/D2 is performed ‘irreducibly’ (i.e., independently of any direct physical interaction between a relativistic observer and a beam of light or quantum probe and target elements) at the HDCR/D2 . . . .

Obviously, the potential computational ramifications of this latter statement regarding the HDCR's objective computational stance may be quite far reaching; In effect, it implies that all space-time and quantum (complimentary) computations are (necessarily) carried out objectively and independently of any direct physical interactions that we may observer (or even initiate as scientists)!

Hence, for instance, in the abovementioned case of ‘quantum entanglement’—in which there exist (two) simultaneous events that are separated by a spatial-temporal distance greater than that possibly traversed by a light beam (between these two events), we must conclude that the HDCR's OCP/D2 determination of the simultaneity of these two events is ‘objective’ (and independent) of any light-beam interacting with these two events. What's essential to understand is that once we abandon the (current) quantum and relativistic materialistic-reductionistic basic working hypothesis (e.g., wherein any space-time or quantum-complimentary phenomenology is assumed to be determined through a direct physical interaction ‘di1’ between a light beam emanating from an object or an event and an observer or between a subatomic probe and target elements), then we are necessarily thrust into the (abovementioned) objective, independent and irreducible hierarchical-dualistic computational reality (HDCR/D2) . . . .

Note, however that the existence of such an objective-irreducible HDCR is not merely a philosophical concept, but in fact bears direct theoretical ramifications relevant for the potential unification of quantum and relativistic models; This is because given the abovementioned impossibility of determining the simultaneity of two entangled events (whose spatial-temporal distance is greater than that possibly traveled by a light beam between them), it becomes clear that the only possible determination of all simultaneous—quantum as well as relativistic—events may be produced by precisely such ‘physically irreducible’ HDCR. In practice this implies that there must exist a such HDCR singular-universal computational frame (SUCF) at the HDCR/D2 level underlying all simultaneous quantum and relativistic objects or events! The reasoning is quite self-explanatory: the fact that simultaneity cannot be determined through a direct physical interaction between beams of light travelling from two such (hypothetical) events and a relativistic observer, e.g., both as proven by the Duality Principle or in the case of quantum (or hypothetically also relativistic) entanglement—necessitates the existence of a conceptually higher-ordered OCP/D2 that is (in principle) irreducible to the ‘di1’ physical interaction between light beams and observers or between subatomic probe and target elements and which can alone determine the existence of any such quantum or relativistic simultaneous (or entangled) events (or objects). Finally, such HDCR independent and irreducible OCP/D2 must necessarily constitute a ‘universal simultaneous computational frame’ (USCF) precisely because of the (abovementioned) conceptual inability to determine all such simultaneous (quantum or relativistic) occurrences through any (other) physical interaction means, which therefore requires OCP/D2 to determine all simultaneous events (or objects) throughout the universe!

The (hypothetical) existence of such universal simultaneous computational frames (USCF) may also bear significant theoretical implications as it immediately raises key questions regarding the HDCR/D2's determination of ‘non-simultaneous’ occurrences; If indeed all simultaneous (quantum or relativistic) events or objects are determined (solely) through a USCF, then this implies that ‘non-simultaneous’ quantum or relativistic events must necessarily belong to disparate USCF's! This is (again) due to the fact that there is no other (physical) means other than through the conceptually higher-ordered OCP/D2 to determine the simultaneity of any two hypothetical spatial-temporal (quantum or relativistic) events, e.g., as belonging to the same USCF—or by contrast OCP/D2's determination of the non-simultaneity of any two hypothetical events as belonging to different USCF's . . . . Obviously, the next key question is: what may be the computational properties (or characteristics) of such disparate USCF's—giving rise to non-simultaneous spatial-temporal events (or objects)? I suggest that based upon the same HDCR/D2 reasoning, e.g., which proves the conceptual computational inability to determine the simultaneity (or non-simultaneity) of any two (hypothetical) spatial-temporal events through ‘di1’ physical interaction between light-beams traveling from these events and a given observer, that we can infer that the OCP/D2 of ‘non-simultaneous’ events must also be determined through another USCF{1}—albeit one that is different than the previous USCF{2}. . . . Again, if it were possible to determine the non-simultaneity of two given spatial temporal events directly through the measurement of light beams traveling from these events to a certain observer then this would necessarily produce a SROCS/SRONCS structure which would produce computational indeterminacy (and logical inconsistency) that are contradicted by empirical measurements of quantum entanglement. Moreover, since (as indicated above) all simultaneous spatial-temporal events must necessarily belong to the same (conceptually higher-ordered) USCF{1}, then we must conclude that any ‘non-simultaneous’ event (i.e., to those contained by USCF{1} have to belong to an immediately following USCF{2}.

I suggest that the (potential) theoretical ramifications of such a hypothetical series of USCF's may be quite significant:

First, since the existence of these (hypothetical) USCF's stems from conceptual computational inability to determine any simultaneous quantum or relativistic phenomena based on the direct physical interaction between an observer and a light beam or between a subatomic probe and target elements (but only through such conceptually higher-ordered USCF's), then also necessarily the determination of any other (relativistic) physical attribute such as time, space, energy or mass or of any other (quantum) complimentary spatial/energetic or temporal/mass, particle/wave feature may only be derived through an analysis of these hypothetical (sequential) USCF's . . . .

Hence, I hypothesize that each one of the abovementioned (quantum or relativistic) physical features may (only) be derived based on an analysis of the sequential USCF's.

Indeed, it should be noted in this context that even the speed of light is not “privileged”—e.g., being equally (computationally) constrained by the conceptually higher-ordered USCF's sequential process; Thus (in contrast to existent relativistic theory) the speed of light itself may not be determined directly though its physical interaction with any observer but rather may only be determined through the abovementioned series of sequential USCF's—wherein the light (photon) is localized at a particular ‘spatial’ point across each one of these USCF's—apparently with no computational ability to exist “in-between” two such (immediately following) quantum frames! Hence, light—as any other hypothetical (localized) physical object (or phenomenon) exists—only as single spatial point within a particular USCF{i}, which apparently undergoes various ‘physical’ modifications, i.e., in terms of its respective spatial localization (and immediately surrounding spatial changes) across subsequent USCF's . . . . Indeed, according to this hypothetical ‘Computational Unified Field Theory’ (CUFT) all physical quantum and relativistic features must be derived as (secondary) phenomenal properties of a singular sequential USCF process (anchored in the basic hierarchical-dualistic computational constraint which negates the possibility of determining any spatial, temporal, energetic, mass or any other quantum complimentary property through direct physical interaction between any hypothetical physical object or event and an observer or subatomic probing element—either within a given USCF or across different USCF's!).

Thus, the Computational Unified Field Theory is based on the hypothetical postulates of the sequential non-continuity of a series of (extremely) rapid USCF's, and their empirical quantification as consisting of quantum {h} based minimal physical differences across USCF's along with a (non-interactive) ‘c’ (square) as representing the (maximal) degree of change across USCF's; Hence, CUFT hypothesizes that an object's measurement of energy (‘e’) is computed on the basis of an OCP/D2 of ‘non-local’ change of the spatial positioning of that object (or event) across immediately subsequent USCF's. Based on Relativity's assertion regarding the constancy of the speed of light as representing the limit for the fastest traveling object, CUFT represents the speed of light ‘c’ as the maximal degree of displacement across subsequent USCF's. Moreover, given the abovementioned conceptual computational proof for the inability to compute this constancy of the speed of light based on its direct physical interaction with any (hypothetical) relativistic observer, the USCF posits that ‘c²’ represents a computational index of the overall (maximal) degree of change across subsequent USCF's (e.g., as ‘c’ represents the greatest/maximal spatial change of a light beam across adjacent USCF's); Hence, CUFT postulates that the rate at which subsequent USCF's are computed (or ‘presented’) can be derived as the ratio of the maximal (overall) degree of change across USCF's, ‘c’ divided by the minimal change in (quantum) energy measurement {‘h’}:

‘c’/‘h’=R{USCFi−USCFi+1}

Next, CUFT posits that since none of the quantum or relativistic physical properties may be derived through a direct interaction between a light-signal and an object, event or subatomic probe and target elements, then all of these quantum or relativistic physical properties must be derived as secondary features arising from the abovementioned USCF serial computational process;

Hence, the CUFT hypothesizes that each of the four abovementioned basic physical properties may be derived based on two conceptually higher-ordered (OCP/D2) computational dimensions which describe the degree of ‘non-local’ ‘change’/‘stability’ of an object across subsequent USCF's, and its number of ‘local’ ‘same’/‘different’ localization across subsequent USCF's;

Thus, CUFT defines ‘time’ as the number of an object's non-local (contextual) ‘un-equal’ presentations across subsequent USCF's divided by the speed of light:

T: ΣO−Li+1{x,y,z} [USCF(n)] ≠O−Ln+1{x,y,z} [USCF(n+1)]/h

Space is the sum of an object's (un-changed) positions across subsequent USCF frames, e.g., such that there exists at least one pair of USCF's for which the object's localization is ‘unchanged’ divided by the minimal degree of spatial displacement ‘h’:

S: O−Li{x,y,z} [USCF(n)])+(O−Lj{x,y,z} [USCF(n+1)])/c, such that: O−Li{x,y,z} [USCF(n)])=(O−Lj{x,y,z} [USCF(n+1)])

‘mass’ is defined as the rate of projection of an object locally (measured from within the object—regardless of where this object appears within subsequent USCF's), divided by Planck's constant:

M: Σ[O−Li{x,y,z}=OLj{x,y,z}] {USCF(n . . . w)}/h

‘energy’ is the difference in an object's localization across subsequent frames divided by the speed of light:

E: (O−Li{x,y,z} [USCF(n)])−(O−Lj{x,y,z} [USCF(n+1)])/c such that O−Li{x,y,z} [USCF(n)])≠(O−Lj{x,y,z} [USCF(n+1)])

I suggest that the elegance of this tentative computational framework is in its broad capacity to both replicate Relativity's and quantum mechanics' known empirical findings, but also go further to potentially unify between these (apparently disparate) theoretical frameworks (and also postulate novel predictions of the CUFT which can be empirically verified);

First, lets examine the potential equivalences between the CUFT's postulates and empirically validated findings of Relativity Theory and Quantum Mechanics: Thus for instance, I suggest that CUFT precisely replicates the findings of the dilation of time for relativistic observers due to the fact that the faster an object travels, the less contextual change occurs! This may sound ‘counter-intuitive’ but when we examine this dictum closely we may realize that as an object approaches the speed of light it ‘dominates’ the contextual (local) points surrounding this object (across subsequent frames). Thus, at the speed of light the light signal constitutes the (immediate) context of the object at any of the serial UCSF's thereby (in effect) cancelling any changes in that object's context (across subsequent USCF's)—thereby in effect halting the progression of time . . . . Another way of representing this is by considering the computation of contextual change (across serial USCF's) which is necessarily relative, i.e., any (hypothetical) object L−{x,y,z}[USCFi . . . n] across multiple USCF's there is potential change which is minimally represented by ‘h’ and maximally represented by ‘c’; Therefore, paradoxically, for a relativistic object (approaching the speed of light) the degree of contextual change must necessarily be close to ‘h’ (but since ‘h’ represents the limit of our ability to measure temporal as well as any other physical changes then once an object reaches the speed of light—time seems to stand still);

Similarly, CUFT postulates can also account for Relativity's tenet regarding the spatial contraction of objects traveling at relativistic speeds; This is because as an object approaches the speed of light the number of spatial points that are a part of the travelling object and which are shared by (at least two) USCF's diminishes—with the limit of the speed of light representing a computational situation in which there is not even a single localized point L-{x,y,z} that is shared by two subsequent USCF's, thereby replicating Relativity's dictum regarding the non-spatial dimensions of a photon . . . .

Likewise, CUFT replicates the relativistic findings regarding the (famous) equivalence of mass and energy: E=Mc²; if we apply this formula to CUFT's definitions of energy and mass we obtain:

E: (O−Li{x,y,z} [USCF(n)])−(O−Lj{x,y,z} [USCF(n+1)])/c=M: Σ[O−Li{x,y,z}=OLj{x,y,z}] {USCF(n . . . w)}×c²/h

First, a development of the above equation yields:

E: (O−Li{x,y,z} [USCF(n)])−(O−Lj{x,y,z} [USCF(n+1)])=M: Σ[O−Li{x,y,z}=OLj{x,y,z}] {USCF(n . . . w)}×c²×(c/h)

It implies that in order to measure an object's ‘energy’ (e.g., the degree of displacement across subsequent USCF's relative to the maximal displacement of the speed of light) we can take that object's ‘mass’ (which corresponds to the number of identical presentations of that object across the series of USCF frames) multiplied by CUFT's basic constant of the rate of universal computational processing (c/h) and then multiplied by Relativity's c². Interestingly, it shows us a very important interrelationship that exists between different (‘internal-stable’: ‘mass’ and ‘external-unstable’: ‘energy’) aspects of the (singular) USCF's computational process, e.g., the fact that the greater the displacement an object has across frames (its ‘energy’), the greater also is the computational derivative corresponding to the number of presentations of that object across these USCF's (its mass). Note, however, that whether or not that object has indeed been presented in each and every one of the USCF's interceding between the two measured USCF's [n, n+1], the computational product representing the hypothesized number of identical presentations of this object increases considerably—i.e., as product of the square of the speed of light.

Within such (relativistic) computational framework note that space-time represents a ‘complimentary’ “local-changing” (time) and “non-local-stable” computational pair, which is interestingly computationally connotes the number of an object's computationally stable points relative to the number of unstable points surrounding that object! One immediate ramification of this (novel) computational formulation of relativistic space-time is that for objects moving at higher velocities their surrounding space will be an object traveling ‘fast’

$$Space \text{-} time \text{:}$$

$$\begin{matrix}S \text{:} \\ T \text{:}\end{matrix} \frac{O \text{-} Li \{ x , y , z \} [ USCF ( n ) ] ) + … ( O \text{-} Lj \{ x , y , z \} [ USCF ( n + l ) ] ) \times c}{\begin{matrix}\sum O \text{-} Li + l \{ x , y , z \} [ USCF ( n ) ] \neq O \text{-} Ln + \\ l \{ x , y , z \} [ USCF ( n + l ) ] \times h\end{matrix}}$$

$$\begin{matrix}Energy \text{-} mass \text{:} \\ \end{matrix} \frac{M \text{:} \sum [ O \text{-} Li \{ x , y , z \} = OLj \{ x , y , z \} ] \{ USCF ( n … w ) \} \times c}{E \text{:} ( O \text{-} Li \{ x , y , z \} [ USCF ( n ) ] ) - ( O \text{-} Lj \{ x , y , z \} [ USCF ( n + l ) ] ) h}$$

The CUFT also postulates that there exists a equivalence between the ‘internal-stable’ (mass)—‘external-unstable’ (energy) complimentary computational pair and the converse ‘internal-unstable’ (time)—‘external-stable’ (space) computational pair, thus:

$$\frac{( O \text{-} Li \{ x , y , z \} [ USCF ( n ) ] ) + ( O \text{-} Lj \{ x , y , z \} [ USCF ( n + l ) ] ) \times c =}{\sum O \text{-} Li + l \{ x , y , z \} [ USCF ( n ) ] \neq O \text{-} Lj + l \{ x , y , z \} [ USCF ( n + l ) ] h}$$

$$\frac{\sum [ O \text{-} Li \{ x , y , z \} = OLj \{ x , y , z \} ] \{ USCF ( n … w ) \} \times c}{( O \text{-} Li \{ x , y , z \} [ USCF ( n ) ] ) - ( O \text{-} Lj \{ x , y , z \} [ USCF ( n + l ) ] ) h}$$

Interestingly enough, such computational equivalence also leads to a generalized computational complimentarity E×S=T×M

that may be equivalent to the quantum complimentarity (e.g., except that this generalized CUFT does not invoke the quantum uncertainty principle). Instead, such computational complimentarity arises simply from CUFT theoretical account of all four physical properties arising as secondary local/non-local and stable vs. non-stable (single spatial-temporal) measurements.

Cosmic Computational Field Theory:

An important formula summarizing the abovementioned Computational Unified Field Theory and/or any potential technological and/or scientific and/or theoretical and/or computational implications and/or applications and/or derivations and/or ramifications etc. is hereby presented:

c²/h×C=[m/t×s/e] whereby the rate of ‘universal simultaneous computational frames’ [USCF—e.g., as measured at a particular spatial and/or temporal and/or mass and/or time value/s and/or measurements] which is represented by the square of the speed of light divided by Planck's constant [c²/h] which is multiplied by the ‘rate of Consciousness’ and/or ‘Consciousness state’ which refers to the degree of mental or consciousness change/s and/or the rate of mental or Consciousness activity—e.g., the number of computational frames that an individual is presented with due to his/her mental and/or cognitive and/or consciousness state (i.e. such as for instance the decrease or even halting or non-presentation in the number of computational frames that are being processed or presented to a person in deep sleep or meditation and/or altered states of Consciousness of any USCF to a person in deep sleep or ‘samadhi’—i.e. mystical state of consciousness)=is equal to the product of the ‘mass’ {m} divided by the time {t} measured at a particular object and/or spatial and/or temporal and/or associated and/or measured at a particular spatial and/or energetic value/s—multiplied by the spatial value/s divided by the energy value/s. This formula implies that all space, time, energy, mass (and causality) values of an object and/or quantum and/or particle-wave measurements can be derived from a unitary singular unified Computational Universal Simultaneous Frames process, which is also individually filtered and/or can be manipulated by the Consciousness computational factor.

Causal Variance

But, if indeed we cannot [in principle] determine the ‘existence’ or ‘nonexistence’ of any ‘yi1’ element—from within its direct interaction with any other ‘xi1’ factor or entity [at the di1 level] but only from a conceptually higher-ordered COP/D2 level, then this necessarily raises serious questions regarding our current [empiricist] scientific conceptualization of ‘causality’, e.g., of a certain ‘x’ entity ‘causing’ [or determining] the existence/non-existence of a certain ‘y’ element; This is because the abovementioned basic constraint set by hierarchical-dualistic computation, namely: CCP/D2≠PR{x,y}→[‘y’ or ‘ [symbol] y’]/di1 conceptually negates our capability to determine the existence of any ‘causal’ relationship between any two interacting ‘x’ and ‘y’ elements—from within their direct interaction [at the di1] level; To prove this lets assume that it is possible to carry out such ‘causal computational processing’ [CCP] that can determine the ‘existence’/‘non-existence’ of a certain ‘causal’ relationship between the ‘x’ and ‘y’ entities—from within their direct physical interaction: CCP/di1=PR{x,y}→[‘y’ or ‘ [symbol] y’]. But, this computational structure precisely replicates that of the [abovementioned] SROCS, which therefore has the potential of expressing a SRONCS that leads to LI/MI and is therefore [once again] constrained by the Duality Principle. Hence, the hierarchical-dualistic computational constraint is conceptual in nature—which implies that it is not possible [in principle] to determine the ‘existence’ or ‘non-existence’ of any ‘causal’ relationship CCP/D2≠PR{x,y}/di1→[‘y’ or ‘ [symbol] y’] not only for a specific ‘x’ or ‘y’ entities but for any PR{xi1, yi1}/di1 computational level. However, this implies that none of the earlier mentioned scientific SROCS, nor any other empirical [or ontological] ‘causal’ relationship, CCP/di1=PR{xi1,yi1}→[‘yi1’ or ‘ [symbol] yi1’]/di1 or can be computed or can be said to ‘exist’ purely at the di1 computational level;

We are therefore forced to accept [once again] a potentially ‘counterintuitive’ conceptual conclusion wherein ‘causality’ does not ‘exist’ [i.e., or at least cannot be determined] at the PR{x,y}/di1 or CR{x,y}/di1 level—but may only ‘exist’ [or be determined] at a higher-ordered CCP/D2. One direct implication of this [novel] assertion is that for each of the earlier mentioned SROCS [including: Darwin's natural selection principle’, the probabilistic interpretation of the quantum uncertainty principle, Relativity's [assumed] measurement of the constancy of the speed of light, G [symbol] del's Incompleteness Theorem, and the neuroscientific materialistic-reductionistic working hypothesis]—there cannot exist any ‘causal’ relationship between the [particular] ‘x’ and ‘y’ elements at the PR/di1 computational level. [Or conversely, no direct causal relationship can be determined between any ‘x’ and ‘y’ entities—purely based on their direct physical interaction at di1.] But, since each of these SROCS/SRONCS scientific paradigms [currently] assumes that the ‘existence’ or ‘non-existence’ of the ‘y’ element [e.g., an organism/specie, the subatomic target's complimentary ‘spatial/energetic’ or ‘mass/temporal’ localization along a probabilistic wave function, the constancy of the speed of light or subthreshold psychophysical stimulation]—is ‘causally’ determined through its direct interaction with another ‘x’ element, then the Duality Principle's conceptual negation of the ability determine any such CCP/D2≠PR{x,y}/di1→[‘y’ or ‘ [symbol] y’] from within the di1 level sharply challenges these scientific SROCS paradigms. More generally, the Duality Principle's conceptual negation of any ‘causal’ [or any other] relationship at the PR/di1 level may call for quite far reaching theoretical [scientific] implications, as it calls for a basic reformalization of all empirical scientific causal [‘x→y’] relationships based on the ‘hierarchical-dualistic computational reality’ which necessarily constrains [and construes] our inductive or deductive scientific knowledge.

Nevertheless [as shown above], the hierarchical-dualistic computational reality proves that the determination of the ‘existence’/‘non-existence’ of any [causal] relationship—may only be carried out at the higher-ordered CCP/D2. So, the remaining question is: what may be a possible causal-computational processing taking place at D2 that may enable a given computational system to determine the ‘existence’/‘non-existence’ of such CCP/D2=[’{x→y}’ or ‘{y→x}’ or ‘{x [symbol] [symbol] y}’]; In order to address this important theoretical question, let us first rule out what type of CCP/D2 may not qualify as the required CCP/D2. It should be noted that based on the [earlier outlined] generalized proof of the Duality Principle—which asserts the conceptual computational irreducibility of CCP/D2 to the direct PR{x,y}/di1, we must reject precisely such direct PR{x,y} interaction as constituting the desired CCP at D2. This is because if the required CCP/D2 would consist of the same type of direct physical interaction that characterized the PR{x,y}/di1 at the di1 level, then it would precisely replicate the SROCS structure and therefore inevitably be constrained by the abovementioned hierarchical-dualistic computational reality—that would [once again] necessitate a conceptually higher-ordered CCP/D2 . . . . Therefore, we must conclude that the determination of any ‘causal’ relationship between any two [or more] given ‘xi1’ and ‘yi1’ [empirical or ontological] elements—may only take place at a conceptually higher-ordered CCP/D2 that is [in principle] ‘irreducible’ to-[and does not consist of-] the direct physical interaction between the ‘xi1’ and ‘yi1’ elements: CCP/D2≠PR{x,y}/di1→[‘y’ or ‘ [symbol] y’].

I hypothesize that another key characteristic of such conceptually higher-ordered CCP/D2 is the [spatial-temporal] ‘non-simultaneity’ of the ‘x’ and ‘y’ elements, e.g., xst{i}≠yst{i+n}. The rational for this CCP/D2 conceptualization of all ‘causal’ relationships stems from the fact that any [potential] ‘causal’ relationship between any [two] given ‘x→y’ elements [or events] necessitates sequential, e.g., non-simultaneous [xst{i}≠yst{i+n}] occurrence of these two elements—since without ‘x’ occurring prior to ‘y’ there could not exist any ‘causal’ relationship between them [‘x→y’]. What's perhaps less evident is the [potentially far reaching] theoretical implications of such [spatial-temporal] ‘non-simultaneity’—i.e., especially when applied to Relativity Theory [and indirectly also to the current formalization of quantum mechanics]; This is because defining a ‘causal’ x→y relationship on the basis of an ‘earlier’ space-time value measured for the ‘x’ than ‘y’ event may [surprisingly] produce ‘causal variance’ in the relationship between the ‘x’ and ‘y’ entities [or events]—as computed by different space-time CCP/D2 [relativistic] systems; In order to prove this [potential] space-time CCP/D2 variance in the ‘causal’ relationship between the xst{i} and yst{i+n} elements lets examine a specific case consisting of two sequential light-beam emission from the abovementioned xst{i} and yst{i+n} events—which is then computed for the existence of potential ‘causal’ relationship by two [uniformly moving] relativistic observers [‘Oa’ and ‘Ob’] that are located on the ‘right’ and ‘left’ side of these two xst{i} and yst{i+n} events [see FIG. 1]. For simplification purposes lets also assume [initially] that these two relativistic observers are traveling in the same direction [e.g., three-dimensional vector] which is from ‘left’ to ‘right’ [i.e., towards the axis of the xst{i} and yst{i+n}, respectively—such that observer ‘Oa’ is approaching the xst{i}−yst{i+n} axis whereas observer ‘Ob’ is receding from these events]. Now, let us all arrange the spatial distance [Sdv{Ys[i]−Xs[i+n]}] between the xst{i} and yst{i+n} events in such a manner that it is precisely synchronized with the time it takes the light beam from xst{i} to reach yst{i+n}, i.e., t{Ys[i]−Xs[i+n]}=Sdv{Ys[i]−Xs[i+n]}/c. In this case, we see that ‘Oa’ measures the [required] ‘non-simultaneous’ [xst{i}≠yst{i+n}] sequence of ‘x’ and ‘y’ events that meets the [earlier mentioned] CCP/D2 requirements for determining a [potential] x→y ‘causal’ relationship. In contrast, I suggest that ‘Ob’ may only measure a spatial-temporal ‘simultaneous’ occurrence of the xst{i} and yst{i+n} events—which is due to the equivalence of the time it takes the light beam to reach from xst{i} to yst{i+n} and the onset of the [‘second’] yst{i+n} light beam. Moreover, to make this illustration of the contingency of any [potential] ‘causal’ relationship between the abovementioned xst{i} and yst{i+n} events [perhaps] even more striking I suggest that if we delayed the synchronization between the xst{i} and yst{i+n} events such that the time it takes the light beam from xst{i} to reach yst{i+n} would be greater than the temporal gap between these two events, i.e., t{Ys[i]−Xs[i+n]}<Sdv{Ys[i]−Xs[i+n]}/c, then ‘Ob’ should even measure a potentially ‘reversed’ causal relationship of y→x . . . . This is because the yst{i+n} light beam reaches ‘Ob’ prior to the xst{i} light beam [thereby potentially producing a CCP/D2=[y→x; yst{i}≠xst{i+n}]. Hence, we see that it is possible to have [potential] ‘causal-variance’ pertaining to the relationship between the xst{i} and yst{i+n} events—based on CCP/D2 performed at different spatial-temporal coordinate systems.

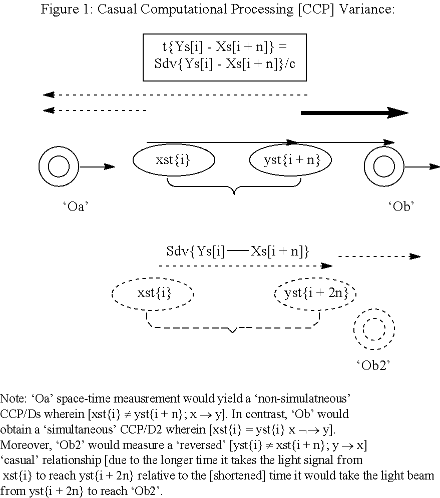

Obviously, such ‘causal-variance’, e.g., of different relativistic observers determining [potentially] different causal relationships between the xst{i}≠yst{i+n} events may have quite far reaching theoretical ramifications, such as for instance in terms of determining the ‘causal’ relationships between events [occurrences or elements] pertaining to the development of our physical universe [e.g., especially as measured by significantly different space-time coordinate relativistic observers]. More conceptually, the existence of CCP/D2 ‘causal-variance’ between certain xst{i} and yst{i+n} events—as computed by different space-time coordinate systems may also go against one of the key postulates of Relativity Theory (Einstein, 1920), namely: the equivalence of the laws of physics for all relativistic space-time coordinate systems. But, given Relativity Theory's robust empirical validation [including its central equivalence principle], then we must recognize the Duality Principle's proven CCP/D2 ‘causal-variance’ as indicating that any x-y ‘causal’ relationship represents only a local [space-time] phenomenon [but may vary across different relativistic observers] . . . Nevertheless, this [novel] CCP/D2 causal-variance postulate would greatly challenge the current materialistic-reductionistic scientific paradigm—as it undermines logical-empirical philosophy of science's [most basic] assumptions pertaining to our ability to determine any ‘causal’ x→y relationship based on empirical measurements of their direct physical interaction, as well as to the ‘causal-invariance’ of any ‘x→y’ relationship [e.g., for all space-time coordinate systems].

More conceptually, the [space-time] ‘non-simultaneity’ of any two given ‘x’ and ‘y’ elements—as a contingency for CCP/D2 of any ‘x→y’ relationship [xst{i}≠yst{i+n}] may further constrain the capacity of the [earlier mentioned] scientific SROCS paradigms to determine the existence/non-existence of the ‘y’ element based on its direct interaction with the particular ‘x’ element [at the di1 level of computation]; This is because the ‘non-simultaneity’ tenet of CCP/D2 asserts the conceptual computational inability to determine any x→y ‘causal’ relationship for any SROCS [scientific] structure—since it implicitly assumes that the COP→[‘y’ or ‘ [symbol] y’] is determined from within the direct [or simultaneous] physical interaction between the ‘x’ and ‘y’ elements [at the di1 level]. Simply stated, the SROCS' simultaneous interaction of the ‘x’ and ‘y’ elements precludes the derivation of any ‘causal’ relationship between them: COP/di1=PR{x,y}→[‘y’ or ‘ [symbol] y’]. This is due to the fact that the CCP tenet necessitates non-simultaneous occurrence of the ‘x’ and ‘y’ elements as a contingency for CCP/D2 determination of the existence of any x→y ‘causal’ relationship. Hence, the SROCS principle structure, which assumes that the determination of the [particular] ‘y’ element is determined [or ‘caused’ by] its direct [‘simultaneous’] interaction with the ‘x’ element—conceptually negates the SROCS'/SRONCS' capability to determine any such CCP/di1=PR{x,y}→[‘y’ or ‘ [symbol] y’]. instead, the [generalized] hierarchical-dualistic computational tenet which asserts the conceptual computational inability to determine the existence of any [particular] ‘y’ element based on its direct [physical] interaction with another ‘x’ element [but only from a higher-ordered CCP/D2 level] points at the inherent ‘insufficiency’ of each of these SROCS structure. But [once again] given each of these SROCS proven empirical capacity to determine: CCP/D2→[‘y’ or ‘ [symbol] y’], the hierarchical-dualistic computational tenet points at the existence of a higher-ordered CCP/D2 [e.g., that is in principle ‘irreducible’ to the PR{x,y}/di1 type of processing]—that [alone] determines the existence/non-existence of the given ‘y’ element. Finally, this conceptual computational inability to determine the existence of any ‘causal’ x→y relationship from within the PR{x,y}/di1 level, along with the [abovementioned] potential CCP/D2 variance of any two ‘x’ and ‘y’ ‘causal’ relationship [as a function of differential space-time coordinate systems] also brings into question the basic notion of ‘causality’ within modern [Cartesian] science.

Single vs. Multiple Spatial-Temporal Measurement/Conceptualization:

In an attempt to weave together the various strands introduced by the Duality Principle and the CUFT it is important to reexamine one further computational dimension that distinguishes between quantum and relativistic modeling, e.g., single vs. multiple measurement or computational perspectives; A closer examination of all four basic abovementioned physical features explicated by (a relativistic) CUFT (e.g., space, time, energy and mass) may indicate that they all arise from computations relating to the displacement- or number of presentations- of a localized (single) spatial-temporal object (or particle). Thus, it may be said that the earlier analysis of CUFT dealt primarily with single spatial-temporal (subatomic particle or relativistic object) computation, which therefore possesses a well-defined ‘spatial’ (localization), ‘mass’, ‘energy’, and ‘temporal’ value—as opposed to the (alternative) multiple spatial-temporal computational characteristics of a subatomic (or macroscopic) ‘wave’ measurement or conceptualization, which are characterized through ‘frequency’, ‘amplitude’, wave-length’ etc. However, I suggest that the mechanism underlying the CUFT, e.g., a phenomenally quick succession of a series of USCF's—with no physical reality ‘in-between’ two such successive frames offers a potential resolution of both the ‘particle/object’ and ‘wave’ characteristics of both quantum and relativistic realms; This is because the same USCF series supports both a localized computation of single spatial-temporal relativistic (particle as well as macroscopic) object as well as a broader multi spatial-temporal wave computation. As a matter of fact, I suggest that quantum entanglement as well as the abovementioned ‘causal variance’ (as well as ‘temporal variance’) are indicative of non-localized ‘wave-like’ computations which broaden (rather than contradict) relativistic (single spatial-temporal) conceptualization.

As such, CUFT has the potential of unifying relativistic and quantum computational modeling—while opening new vistas towards exploring a conceptually higher-ordered (physically irreducible) computational framework. Indeed, further exploration of the CUFT's basic process of USCF presentations may predict a series of yet undiscovered phenomenal features such as an exhaustive computational description of (localized single spatial-temporal) physical reality as a complimentary pairing of local/global and changing/stable physical features, e.g., from which may be derived both the (abovementioned) complimentarity of spatial-energetic and temporal-mass (subatomic as well as relativistic) features as well as the computational equivalence of complimentary internal/external stable/changing (e.g., space-time and energy-mass) measures; But, perhaps the key concept raised by CUFT is its contingency of all physical (relativistic as well quantum) phenomena upon a series of computational USCF's which are (in principle) irreducible to any (direct) physical measurement or interaction—but rather exist as a higher-ordered organizing principle. As such, it becomes clear that any capacity to alter (e.g., locally or non-locally) the rate or displacement characteristics of such conceptually higher-ordered USCF's may lead to a series of (yet undiscovered) physical predictions (all of which may be derived once we gain a clearer understanding of the locus and nature of this higher-ordered USCF process).

1-109. (canceled)

110. A therapeutic system comprising:
a) an electrode implantable in a brain of a patient, said electrode comprises:
i) a signal generator operable to deliver pulsed electromagnetic signals to a local region of said brain;
ii) a sensor that senses electromagnetic brain activity in said local region;
iii) a processor programmed to adjust said deliverable electromagnetic signals as a function of said sensed brain activity; and
b) a device external to said patient which provides sensory stimulation to said patient, said stimulation being timed to coordinate with timing of delivery of said electromagnetic signal by said signal generator of said implanted electrode.
